SEQUENCE LISTING (509 sequences)

SEQ ID NO: 1 - protein, 117 aa, Homo Sapiens
EVQLLESGGGLVQPGGSLRLSCAASGFTFSDYWMSWVRQAPGKGLEWVSSISTSSSYIYYADSVKGRFTISRDNSKNTLYLQMNSLRAEDTAVYYCARVWRNYLDYWGQGTLVTVSS

SEQ ID NO: 2 - protein, 110 aa, Homo Sapiens
QSVLTQPPSASGTPGQRVTISCSGSSSNIGSNKVNWYQQLPGTAPKSLIWSNNQRPSGVPDRFSGSKSGTSASLAITGLQAEDEADYYCQSYDSSLSGWVFGGGTKLTVL

SEQ ID NO: 3 - protein, 117 aa, Homo Sapiens; identical to SEQ ID NO: 1

SEQ ID NO: 4 - protein, 110 aa, Homo Sapiens
QSVLTQPPSASGTPGQRVTISCSGSSSNIGSNKVNWYQQLPGTAPKVLIWSNNQRPSGVPDRFSGSKSGTSASLAITGLQAEDEADYYCQSYDSSLVGWVFGGGTKLTVL

SEQ ID NO: 5 - protein, 117 aa, Homo Sapiens
EVQLLESGGGLVQPGGSLRLSCAASGFTFSDYWMSWVRQAPGKGLEWVSSISSSSSYIYYADSVKGRFTISRDNSKNTLYLQMNSLRAEDTAVYYCARVWRNYLSYWGQGTLVTVSS

SEQ ID NO: 6 - protein, 110 aa, Homo Sapiens
QSVLTQPPSASGTPGQRVTISCSGSSSNIGSNKVNWYQQLPGTAPKVLIWSNNQRPSGVPDRFSGSKSGTSASLAITGLQAEDEADYYCQSYDSSLSGWVFGGGTKLTVL

SEQ ID NO: 7 - protein, 117 aa, Homo Sapiens
EVQLLESGGGLVQPGGSLRLSCAASGFTFSDYWMSWVRQAPGKGLEWVSSISTSSSYIYYADSVKGRFTISRDNSKNTLYLQMNSLRAEDTAVYYCARVWRNYLSYWGQGTLVTVSS

SEQ ID NO: 8 - protein, 110 aa, Homo Sapiens; identical to SEQ ID NO: 6

SEQ ID NO: 9 - protein, 117 aa, Homo Sapiens; identical to SEQ ID NO: 5

SEQ ID NO: 10 - protein, 110 aa, Homo Sapiens
QSVLTQPPSASGTPGQRVTISCSGSSSNIGSNKVNWYQQLPGTAPKSLIYSNNQRPSGVPDRFSGSKSGTSASLAITGLQAEDEADYYCQSYSSSLSGWVFGGGTKLTVL

SEQ ID NO: 11 - protein, 117 aa, Homo Sapiens; identical to SEQ ID NO: 7

SEQ ID NO: 12 - protein, 110 aa, Homo Sapiens
QSVLTQPPSASGTPGQRVTISCSGSSSNIGSNKVNWYQQLPGTAPKSLIYSNNQRPSGVPDRFSGSKSGTSASLAITGLQAEDEADYYCQSYSSSLVGWVFGGGTKLTVL

SEQ ID NO: 13 - protein, 117 aa, Homo Sapiens; identical to SEQ ID NO: 5

SEQ ID NO: 14 - protein, 110 aa, Homo Sapiens
QSVLTQPPSASGTPGQRVTISCSGSSSNIGSNKVNWYQQLPGTAPKSLIWSNNQRPSGVPDRFSGSKSGTSASLAITGLQAEDEADYYCQSYDSSLVGWVFGGGTKLTVL

SEQ ID NO: 15 - protein, 117 aa, Homo Sapiens; identical to SEQ ID NO: 7

SEQ ID NO: 16 - protein, 110 aa, Homo Sapiens
QSVLTQPPSASGTPGQRVTISCSGSSSNIGSNKVNWYQQLPGTAPKSLIWSNNQRPSGVPDRFSGSKSGTSASLAITGLQAEDEADYYCQSYSSSLSGWVFGGGTKLTVL

SEQ ID NO: 17 - protein, 117 aa, Homo Sapiens; identical to SEQ ID NO: 1

SEQ ID NO: 18 - protein, 110 aa, Homo Sapiens
QSVLTQPPSASGTPGQRVTISCSGSSSNIGSNKVNWYQQLPGTSPKILIWSNNQRPSGVPDRFSGSKSGTSASLAITGLQAEDEADYYCQSYSSSLVGWVFGGGTKLTVL

SEQ ID NO: 19 - protein, 117 aa, Homo Sapiens
EVQLLESGGGLVQPGGSLRLSCAASGFTFSDSWMSWVRQAPGKGLEWVSSISTSSSYIYYADSVKGRFTISRDNSKNTLYLQMNSLRAEDTAVYYCARVWRNSLDYWGQGTLVTVSS

SEQ ID NO: 20 - protein, 110 aa, Homo Sapiens
QSVLTQPPSASGTPGQRVTISCSGSSSNIGSNKVNWYQQLPGTAPKLLIWSNNQRPSGVPDRFSGSKSGTSASLAITGLQAEDEADYYCQSYDSSLVGWVFGGGTKLTVL

SEQ ID NO: 21 - protein, 117 aa, Homo Sapiens; identical to SEQ ID NO: 19

SEQ ID NO: 22 - protein, 110 aa, Homo Sapiens
QSVLTQPPSASGTPGQRVTISCSGSSSNIGSNKVNWYQQLPGTAPKLLIWSNNQRPSGVPDRFSGSKSGTSASLAITGLQAEDEADYYCQSYDSSLSGWVFGGGTKLTVL

SEQ ID NO: 23 - protein, 117 aa, Homo Sapiens; identical to SEQ ID NO: 7

SEQ ID NO: 24 - protein, 110 aa, Homo Sapiens
QSVLTQPPSASGTPGQRVTISCSGSSSNIGSNTVNWYQQLPGTAPKVLIWSNNQRPSGVPDRFSGSKSGTSASLAITGLQAEDEADYYCQSYSSSLSGWVFGGGTKLTVL

SEQ ID NO: 25 - protein, 117 aa, Homo Sapiens; identical to SEQ ID NO: 5

SEQ ID NO: 26 - protein, 110 aa, Homo Sapiens
QSVLTQPPSASGTPGQRVTISCSGSSSNIGSNTVNWYQQLPGTAPKSLIYSNNQRPSGVPDRFSGSKSGTSASLAITGLQAEDEADYYCQSYSSSLSGWVFGGGTKLTVL

SEQ ID NO: 27 - protein, 117 aa, Homo Sapiens; identical to SEQ ID NO: 5

SEQ ID NO: 28 - protein, 110 aa, Homo Sapiens
QSVLTQPPSASGTPGQRVTISCSGSSSNIGSNKVNWYQQLPGTAPKVLIWSNNQHPSGVPDRFSGSKSGTSASLAITGLQAEDEADYYCQSYDSSLVGWVFGGGTKLTVL

SEQ ID NO: 29 - protein, 117 aa, Homo Sapiens
EVQLLESSGGLVQPGGSLRLSCAASGFTFSDYWMSWVRQAPGKGLEWVSSISSSSSYIYYADSVKGRFTISRDNSKNTLYLQMNSLRAEDTAVYYCARVWRNSLSYWGQGTLVTVSS

SEQ ID NO: 30 - protein, 110 aa, Homo Sapiens; identical to SEQ ID NO: 26

SEQ ID NO: 31 - protein, 117 aa, Homo Sapiens
EVQLLESGGGLVQPGGSLRLSCAASGFTFSDYWMSWVRQAPGKGLEWVSSISTSSSYIYYADGVKGRFTISRDNSKNTLYLQMNSLRAEDTAVYYCARVWRNYLSYWGQGTLVTVSS

SEQ ID NO: 32 - protein, 110 aa, Homo Sapiens
QSVLTQPPSASGTPGQRVTISCSGSSSNIGSNKVNWYQQLPGTAPKILIWSNNQRPSGVPDRFSGSKSGTSASLAITGLQAEDEADYYCQSYDSSLSGWVFGGGTKLTVL

SEQ ID NO: 33 - protein, 117 aa, Homo Sapiens; identical to SEQ ID NO: 5

SEQ ID NO: 34 - protein, 110 aa, Homo Sapiens
QSVLTQPPSASGTPGQRVTISCSGSSSNIGSNKVNWYQQLPGTAPKVLIYSNNQRPSGVPDRFSGSKSGTSASLAITGLQAEDEADYYCQSYSSSLVGWVFGGGTKLTVL

SEQ ID NO: 35 - protein, 117 aa, Homo Sapiens; identical to SEQ ID NO: 7

SEQ ID NO: 36 - protein, 110 aa, Homo Sapiens
QSVLTQPPSASGTPGQRVTISCSGSSSNIGSNKVNWYQQLPGTAPKVLIYSNNQRPSGVPDRFSGSKSGTSASLAITGLQAEDEADYYCQSYDSSLVGWVFGGGTKLTVL

SEQ ID NO: 37 - protein, 117 aa, Homo Sapiens; identical to SEQ ID NO: 5

SEQ ID NO: 38 - protein, 110 aa, Homo Sapiens
QSVLTQPPSASGTPGQRVTISCSGSSSNIGSNKVNWYQQLPGTAPKSLIWSNNQRPNGVPDRFSGSKSGTSASLAITGLQAEDEADYYCQSYDSSLSGWVFGGGTKLTVL

SEQ ID NO: 39 - protein, 117 aa, Homo Sapiens
EVQLLESGGGLVQPGGSLRLSCAASGFTFSDYWMSWVRQAPGKGLEWVSSISSSSSYIYYADSVKGRFTISRDNSKNTLYLQMNSLRAEDTAVYYCARVWRNYLDYWGQGTLVTVSS

SEQ ID NO: 40 - protein, 110 aa, Homo Sapiens
QSVLTQPPSASGTPGQRVTISCSGSSSNIGSNKVNWYQQLPGTAPKILIWSNNQRPSGVPDRFSGSKSGTSASLAITGLQAEDEADYYCQSYSSSLVGWVFGGGTKLTVL

SEQ ID NO: 41 - protein, 117 aa, Homo Sapiens; identical to SEQ ID NO: 7

SEQ ID NO: 42 - protein, 110 aa, Homo Sapiens; identical to SEQ ID NO: 10

SEQ ID NO: 43 - protein, 117 aa, Homo Sapiens; identical to SEQ ID NO: 1

SEQ ID NO: 44 - protein, 110 aa, Homo Sapiens
QSVLTQPPSASGTPGQRVTISCSGSSSNIGSNTVNWYQQLPGTAPKSLIWSNNQRPSGVPDRFSGSKSGTSASLAITGLQAEDEADYYCQSYSSSLSGWVFGGGTKLTVL

SEQ ID NO: 45 - protein, 117 aa, Homo Sapiens; identical to SEQ ID NO: 1

SEQ ID NO: 46 - protein, 110 aa, Homo Sapiens; identical to SEQ ID NO: 44

SEQ ID NO: 47 - protein, 117 aa, Homo Sapiens; identical to SEQ ID NO: 5

SEQ ID NO: 48 - protein, 110 aa, Homo Sapiens
QSVLTQPPSASGTPGQRVTISCSGSSSNIGSNKVNWYQQLPGTAPKSLIYSNNQRPSGVPDRFSGSKSGTSASLAITGLQAEDEADYYCQSYDSSLVGWVFGGGTKLTVL

SEQ ID NO: 49 - protein, 117 aa, Homo Sapiens; identical to SEQ ID NO: 7

SEQ ID NO: 50 - protein, 110 aa, Homo Sapiens
QSVLTQPPSASGTPGQRVTISCSGSSSNIGSNKVNWYQQLPGTAPKILIWSNNQRPSGVPDRFSGSKSGTSASLAITGLQAEDEADYYCQSYSSSLSGWVFGGGTKLTVL

SEQ ID NO: 51 - protein, 117 aa, Homo Sapiens; identical to SEQ ID NO: 5

SEQ ID NO: 52 - protein, 110 aa, Homo Sapiens
QSVLTQPPSASGTPGQRVTISCSGSSSNIGSNKVNWYQQLPGTAPKVLIWSNNQRPSGVPDRFSGSKSGTSASLAITGLQAEDEADYYCQSYSSSLVGWVFGGGTKLTVL

SEQ ID NO: 53 - protein, 117 aa, Homo Sapiens; identical to SEQ ID NO: 1

SEQ ID NO: 54 - protein, 110 aa, Homo Sapiens
QSVLTQPPSASGTPGQRVTISCSGSSSNIGSNKVNWYQQLPGTAPKSLIWSNNQRPSGVPDRFSGSKSGTSASLAITGLQAEDEADYYCQSYSSSLVGWVFGGGTKLTVL

SEQ ID NO: 55 - protein, 117 aa, Homo Sapiens; identical to SEQ ID NO: 5

SEQ ID NO: 56 - protein, 110 aa, Homo Sapiens
QSVLTQPPSTSGTPGQRVTISCSGSSSNIGSNTVNWYQQLPGTAPKVLIYSNNQRPSGVPDRFSGSKSGTSASLAITGLQAEDEADYYCQSYSSSLSGWVFGGGTKLTVL

SEQ ID NO: 57 - protein, 117 aa, Homo Sapiens
EVQLLESGGGLVQPGGSLRLSCAASGFTFSDYWMSWVRQAPGKGLEWVSSISTSSSYIYYADSVKGRFTISRDNSKNTLYLQMNSLRAEDTAVYYCARVWRNALDYWGQGTLVTVSS

SEQ ID NO: 58 - protein, 110 aa, Homo Sapiens
QSVLTQPPSASGTPGQRVTISCSGSSSNIGSNKVNWYQQLPGTAPKLLIYSNNQRPSGVPDRFSGSKSGTSASLAITGLQAEDEADYYCQSYSSSLSGWVFGGGTKLTVL

SEQ ID NO: 59 - protein, 117 aa, Homo Sapiens
EVQLLESGGGLVQPGGSLRLSCAASGFTFSDSWMSWVRQAPGKGLEWVSSISTSSSYIYYADSVKGRFTISRDNSKNTLYLQMNSLRAEDTAVYYCARVWRNALDYWGQGTLVTVSS

SEQ ID NO: 60 - protein, 110 aa, Homo Sapiens
QSVLTQPPSASGTPGQRVTISCSGSSSNIGSNTVNWYQQLPGTAPKLLIWSNNQRPSGVPDRFSGSKSGTSASLAITGLQAEDEADYYCQSYSSSLVGWVFGGGTKLTVL

SEQ ID NO: 61 - protein, 117 aa, Homo Sapiens; identical to SEQ ID NO: 5

SEQ ID NO: 62 - protein, 110 aa, Homo Sapiens; identical to SEQ ID NO: 40

SEQ ID NO: 63 - protein, 117 aa, Homo Sapiens; identical to SEQ ID NO: 7

SEQ ID NO: 64 - protein, 110 aa, Homo Sapiens
QSVLTQPPSASGTPGQRVTISCSGSSSNIGSNKVNWYQQLPGTAPKVLIWSNNQRPSSVPDRFSGSKSGTSASLAITGLQAEDEADYYCQSYSSSLSGWVFGGGTKLTVL

SEQ ID NO: 65 - protein, 117 aa, Homo Sapiens
EVQLLESGGGLVQTGGSLRLSCAASGFTFSDYWMSWVRQAPGKGLEWVSSISTSSSYIYYADSVKGRFTISRDNSKNTLYLQMNSLRAEDTAVYYCARVWRNYLSYWGQGTLVTVSS

SEQ ID NO: 66 - protein, 110 aa, Homo Sapiens; identical to SEQ ID NO: 52

SEQ ID NO: 67 - protein, 117 aa, Homo Sapiens; identical to SEQ ID NO: 7

SEQ ID NO: 68 - protein, 110 aa, Homo Sapiens; identical to SEQ ID NO: 14

SEQ ID NO: 69 - protein, 117 aa, Homo Sapiens; identical to SEQ ID NO: 7

SEQ ID NO: 70 - protein, 110 aa, Homo Sapiens
QSVLTQPPSASGTPGQRVTISCSGSSSNIGSNTVNWYQQLPGTAPKILIWSNNQRPSGVPDRFSGSKSGTSASLAITGLQAEDEADYYCQSYSSSLSGWVFGGGTKLTVL

SEQ ID NO: 71 - protein, 117 aa, Homo Sapiens
EVQLLESGGGLVQPGGSLRLSCAASGFTFSDSWMSWVRQAPGKGLEWVSSISTSSSYIYYADSVKGRFTISRDNSKNTLYLQMNSLRAEDTAVYYCARVWRNYLDYWGQGTLVTVSS

SEQ ID NO: 72 - protein, 108 aa, Homo Sapiens
QSVLTQPPSASGTPGQRVTISCSGSSSNIGSNKVNWYQQLPGTAPKSLIWSNNQRPSGVPDRFSGSKSGTSASLAITGLQAEDEADYYCQSYSSSLSGWVFGGGTKLT

SEQ ID NO: 73 - protein, 117 aa, Homo Sapiens; identical to SEQ ID NO: 7

SEQ ID NO: 74 - protein, 110 aa, Homo Sapiens
QSVLTQPPSASGTPGQRVTISCSGSSSNIGSNKVNWYQQLPGTAPKVLIYSNNQRPNGVPDRFSGSKSGTSASLAITGLQAEDEADYYCQSYSSSLSGWVFGGGTKLTVL

SEQ ID NO: 75 - protein, 117 aa, Homo Sapiens; identical to SEQ ID NO: 7

SEQ ID NO: 76 - protein, 110 aa, Homo Sapiens; identical to SEQ ID NO: 32

SEQ ID NO: 77 - protein, 117 aa, Homo Sapiens
EVQLLESGGGLVQPGGSLRLSCAASGFTFSDHWMSWVRQAPGKGLEWVSSISTSSSYIYYADSVKGRFTISRDNSKNTLYLQMNSLRAEDTAVYYCARVWRNYLSYWGQGTLVTVSS

SEQ ID NO: 78 - protein, 110 aa, Homo Sapiens; identical to SEQ ID NO: 4

SEQ ID NO: 79 - protein, 117 aa, Homo Sapiens; identical to SEQ ID NO: 5

SEQ ID NO: 80 - protein, 110 aa, Homo Sapiens; identical to SEQ ID NO: 54

SEQ ID NO: 81 - protein, 117 aa, Homo Sapiens
EVQLLESGGGLVQPGGSLRLSCAASGFTFSDYWMSWVRQAPGKGLEWVSSISTSSSYIYYADSVKGRFTISRDNSKNTLYLQMNSLRAEDTAVYYCARVWRNALSYWGQGTLVTVSS

SEQ ID NO: 82 - protein, 110 aa, Homo Sapiens
QSVLTQPPSASGTPGQRVTISCSGSSSNIGSNTVNWYQQLPGTAPKLLIWSNNQRPSGVPDRFSGSKSGTSASLAITGLQAEDEADYYCQSYDSSLSGWVFGGGTKLTVL

SEQ ID NO: 83 - protein, 117 aa, Homo Sapiens; identical to SEQ ID NO: 7

SEQ ID NO: 84 - protein, 110 aa, Homo Sapiens
QSVLTQPPSASGTPGQRVTISCSGSSSNIGSNTVNWYQQLPGTAPKSLIYSNNQRPSGVPDRFSGSKSGTSASLAITGLQAEDEADYYCQSYSSSLVGWVFGGGTKLTVL

SEQ ID NO: 85 - protein, 117 aa, Homo Sapiens; identical to SEQ ID NO: 39

SEQ ID NO: 86 - protein, 110 aa, Homo Sapiens
QSVLTQPPSASGTPGQRVTISCSGSSSNIGSNTVNWYQQLPGTAPKVLIWSNNQRPSGVPDRFSGSKSGTSASLAITGLQAEDEADYYCQSYSSSLVGWVFGGGTKLTVL

SEQ ID NO: 87 - protein, 117 aa, Homo Sapiens; identical to SEQ ID NO: 81

SEQ ID NO: 88 - protein, 110 aa, Homo Sapiens
QSVLTQPPSASGTPGQRVTISCSGSSSNIGSNTVNWYQQLPGTAPKILIWSNNQRPSGVPDRFSGSKSGTSASLAITGLQAEDEADYYCQSYSSSLVGWVFGGGTKLTVL

SEQ ID NO: 89 - protein, 117 aa, Homo Sapiens; identical to SEQ ID NO: 7

SEQ ID NO: 90 - protein, 110 aa, Homo Sapiens
QSVLTQPPSASGTPGQRVTISCSGSSSNIGSNKVNWYQQLPGTAPKSLIYSNNQRPSGVPDRFSGSKSGTSASLAITGLQAEDEADYYCQSYDSSLSGWVFGGGTKLTVL

SEQ ID NO: 91 - protein, 117 aa, Homo Sapiens
EVQLLESGGGLVQPGGSLRLSCAASGFTFSDYWMSWVRQAPGKGLEWVSSISTSSSYIYYADSVKGRFTISRDNSKNTLYLQMNSLRAEDTAVYYCARVWRNSLSYWGQGTLVTVSS

SEQ ID NO: 92 - protein, 110 aa, Homo Sapiens; identical to SEQ ID NO: 12

SEQ ID NO: 93 - protein, 117 aa, Homo Sapiens; identical to SEQ ID NO: 7

SEQ ID NO: 94 - protein, 110 aa, Homo Sapiens; identical to SEQ ID NO: 40

SEQ ID NO: 95 - protein, 117 aa, Homo Sapiens; identical to SEQ ID NO: 1

SEQ ID NO: 96 - protein, 110 aa, Homo Sapiens; identical to SEQ ID NO: 34

SEQ ID NO: 97 - protein, 117 aa, Homo Sapiens; identical to SEQ ID NO: 7

SEQ ID NO: 98 - protein, 110 aa, Homo Sapiens; identical to SEQ ID NO: 2

SEQ ID NO: 99 - protein, 117 aa, Homo Sapiens; identical to SEQ ID NO: 1

SEQ ID NO: 100 - protein, 110 aa, Homo Sapiens; identical to SEQ ID NO: 40

SEQ ID NO: 101 - protein, 117 aa, Homo Sapiens; identical to SEQ ID NO: 7

SEQ ID NO: 102 - protein, 110 aa, Homo Sapiens; identical to SEQ ID NO: 70

SEQ ID NO: 103 - protein, 117 aa, Homo Sapiens
EVQLLESGGGLVQPGGSLRLSCAASGFTFSSYWMSWVRQAPGKGLEWVSSISSSSSYIYYADSVKGRFTISRDNSKNTLYLQMNSLRAEDTAVYYCARVWRNSLDYWGQGTLVTVSS

SEQ ID NO: 104 - protein, 110 aa, Homo Sapiens
QSVLTQPPSASGTPGQRVTISCSGSSSNIGSNTVNWYQQLPGTAPKLLIYSNNQRPSGVPDRFSGSKSGTSASLAITGLQAEDEADYYCQSYDSSLVGWVFGGGTKLTVL

SEQ ID NO: 105 - protein, 117 aa, Homo Sapiens; identical to SEQ ID NO: 103

SEQ ID NO: 106 - protein, 110 aa, Homo Sapiens
QSVLTQPPSASGTPGQRVTISCSGSSSNIGSNTVNWYQQLPGTAPKLLIYSNNQRPSGVPDRFSGSKSGTSASLAITGLQAEDEADYYCQSYDSSLSGWVFGGGTKLTVL

SEQ ID NO: 107 - protein, 117 aa, Homo Sapiens
EVQLLESGGGLVQPGGSLRLSCAASGFTFSSYWMSWVRQAPGKGLEWVSSISSSSSYIYYADSVKGRFTISRDNSKNTLYLQMNSLRAEDTAVYYCARVWRNYLDYWGQGTLVTVSS

SEQ ID NO: 108 - protein, 110 aa, Homo Sapiens; identical to SEQ ID NO: 104

SEQ ID NO: 109 - protein, 117 aa, Homo Sapiens
EVQLLESGGGLVQPGGSLRLSCAASGFTFSNYWMSWVRQAPGKGLEWVSSISSSSGYIYYADSVKGRFTISRDNSKNTLYLQMNSLRAEDTAVYYCARVWRNYLDYWGQGTLVTVSS

SEQ ID NO: 110 - protein, 110 aa, Homo Sapiens; identical to SEQ ID NO: 106

SEQ ID NO: 111 - protein, 117 aa, Homo Sapiens; identical to SEQ ID NO: 109

SEQ ID NO: 112 - protein, 110 aa, Homo Sapiens; identical to SEQ ID NO: 104

SEQ ID NO: 113 - protein, 117 aa, Homo Sapiens
EVQLLESGGGLVQPGGSLRLSCAASGFTFSNYWMSWVRQAPGKGLEWVSSISSSSGYIYYADSVKGRFTISRDNSKNTLYLQMNSLRAEDTAVYYCARVWRNSLDYWGQGTLVTVSS

SEQ ID NO: 114 - protein, 110 aa, Homo Sapiens; identical to SEQ ID NO: 104

SEQ ID NO: 115 - protein, 117 aa, Homo Sapiens; identical to SEQ ID NO: 113

SEQ ID NO: 116 - protein, 110 aa, Homo Sapiens; identical to SEQ ID NO: 106

SEQ ID NO: 117 - protein, 117 aa, Homo Sapiens; identical to SEQ ID NO: 107

SEQ ID NO: 118 - protein, 110 aa, Homo Sapiens; identical to SEQ ID NO: 106

SEQ ID NO: 119 - protein, 117 aa, Homo Sapiens; identical to SEQ ID NO: 109

SEQ ID NO: 120 - protein, 110 aa, Homo Sapiens
QSVLTQPPSASGTPGQRVTISCSGSSSDIGSNTVNWYQQLPGTAPKLLIYDNNQRPSGVPDRFSGSKSGTSASLAITGLQAEDEADYYCQSYDSSLSGWVFGGGTKLTVL

SEQ ID NO: 121 - protein, 117 aa, Homo Sapiens; identical to SEQ ID NO: 107

SEQ ID NO: 122 - protein, 110 aa, Homo Sapiens; identical to SEQ ID NO: 120

SEQ ID NO: 123 - protein, 117 aa, Homo Sapiens; identical to SEQ ID NO: 107

SEQ ID NO: 124 - protein, 110 aa, Homo Sapiens
QSVLTQPPSASGTPGQRVTISCSGSSSDIGSNTVNWYQQLPGTAPKLLIYDNNQRPSGVPDRFSGSKSGTSASLAISGLRSEDEADYYCQSYDSSLSGWVFGGGTKLTVL

SEQ ID NO: 125 - protein, 117 aa, Homo Sapiens
EVQLLESGGGLVQPGGSLRLSCAASGFTFSNAWMSWVRQAPGKGLEWVSSISSSSGYIYYADSLKGRFTISRDNSKNTLYLQMNSLRAEDTAVYYCARVWRNHLDYWGQGTLVTVTS

SEQ ID NO: 126 - protein, 110 aa, Homo Sapiens; identical to SEQ ID NO: 106

SEQ ID NO: 127 - protein, 119 aa, Homo Sapiens
EVQLLESGGGLVQPGGSLRLSCAASGFRFSSYAMSWVRQAPGKGLEWVGGISGSGGSTYYADSVKGRFTISRDNSKNTLYLQMNSLRAEDTAVYYCARQGRTSFYLDVWGQGTLVTVSS

SEQ ID NO: 128 - protein, 111 aa, Homo Sapiens
QSVLTQPPSASGTPGQRVTISCSGSSRNIGSFYVYWYQQLPGTAPKLLIYDNNQRPSGVPDRFSGSKSGTSASLAISGLRSEDEADYYCVAWDDSWSGTWVFGGGTKLTVL

SEQ ID NO: 129 - protein, 119 aa, Homo Sapiens
EVQLLESGGGLVQPGGSLRLSCAASGFTFSSYAMSWVRQAPGKGLEWVGGISGSGGSTYYADSVKGRFTISRDNSKNTLYLQMNSLRAEDTAVYYCARQGRTSFYLDVWGQGTLVTVSS

SEQ ID NO: 130 - protein, 111 aa, Homo Sapiens
QSVLTQPPSASGTPGQRVTISCCGSSRNIGSYYVYWYQQLPGTAPKLLIYDNNQRPSGVPDRFSGSKSGTSASLAISGLRSEDEADYYCVAWDDSWSGTWVFGGGTKLTVL

SEQ ID NO: 131 - protein, 119 aa, Homo Sapiens
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCARQGRTSFYLDVWGQGTLVTVSS

SEQ ID NO: 132 - protein, 111 aa, Homo Sapiens
QSVLTQPPSASGTPGQRVTISCSGSSRNIGSYYVYWYQQLPGTAPKLLIYDNRQRPSGVPDRFSGSKSGTSASLAISGLRSEDEADYYCVAWDDSWSGTWVFGGGTKLTVL

SEQ ID NO: 133 - protein, 119 aa, Homo Sapiens; identical to SEQ ID NO: 127

SEQ ID NO: 134 - protein, 111 aa, Homo Sapiens
QSVLTQPPSASGTPGQRVTISCSGSSRNIGAYYVYWYQQLPGTAPKLLIYDNRQRPSGVPDRFSGSKSGTSASLAISGLRSEDEADYYCVAWDDSYSGTWVFGGGTKLTVL

SEQ ID NO: 135 - protein, 119 aa, Homo Sapiens
EVQLLESGGGLVQPGGSLRLSCAASGFRFSSYAMSWVRQAPGKGLEWVSGISGSGGSTYYADSVKGRFTISRDNSKNTLYLQMNSLRAEDTAVYYCARQGRTSFYLDVWGQGTLVTVSS

SEQ ID NO: 136 - protein, 111 aa, Homo Sapiens
QSVLTQPPSASGTPGQRVTISCSGSSRNIGSYYVYWYQQLPGTAPKLLIYDNRQRPSGVPDRFSGSKSGTSASLAISGLRSEDEADYYCVAWDDSYSGTWVFGGGTKLTVL

SEQ ID NO: 137 - protein, 119 aa, Homo Sapiens
EVQLLESGGGLVQPGGSLRLSCAASGFRFSSYAMSWIRQAPGKGLEWVGGISGSGGSTYYADSVKGRFTISRDNSKNTLYLQMNSLRAEDTAVYYCARQGRTSFYLDVWGQGTLVTVSS

SEQ ID NO: 138 - protein, 111 aa, Homo Sapiens; identical to SEQ ID NO: 136

SEQ ID NO: 139 - protein, 119 aa, Homo Sapiens; identical to SEQ ID NO: 127

SEQ ID NO: 140 - protein, 111 aa, Homo Sapiens
QSVLTQPPSASGTPGQRVTISCSGSSRNIGSYYVYWYQQLPGTAPKLLIYDNNQRPSGVPDRFSGSKSGTSASLAISGLRSEDEADYYCVAWDDSWSGHWVFGGGTKLTVL

SEQ ID NO: 141 - protein, 119 aa, Homo Sapiens; identical to SEQ ID NO: 129

SEQ ID NO: 142 - protein, 111 aa, Homo Sapiens
QSVLTQPPSASGTPGQRVTISCSGSSRNIGAFYVYWYQQLPGTAPKLLIYDNRQRPSGVPDRFSGSKSGTSASLAISGLRSEDEADYYCVAWDDSYSGTWVFGGGTKLTVL

SEQ ID NO: 143 - protein, 119 aa, Homo Sapiens; identical to SEQ ID NO: 127

SEQ ID NO: 144 - protein, 111 aa, Homo Sapiens
QSVLTQPPSASGTPGQRVTISCSGSSRNIGSFYVYWYQQLPGTAPKLLIYDNNQRPSGVPDRFSGSKSGTSASLAISGLRSEDEADYYCVAWDVSLSGHWVFGGGTKLTVL

SEQ ID NO: 145 - protein, 119 aa, Homo Sapiens; identical to SEQ ID NO: 129

SEQ ID NO: 146 - protein, 111 aa, Homo Sapiens
QSVLTQPPSASGTPGQRVTISCSGSSRNIGSFYVYWYQQLPGTAPKLLIYDNNQRPSGVPDRFSGSKSGTSASLAISGLRSEDEADYYCVAWDDSWSGHWVFGGGTKLTVL

SEQ ID NO: 147 - protein, 119 aa, Homo Sapiens; identical to SEQ ID NO: 127

SEQ ID NO: 148 - protein, 111 aa, Homo Sapiens
QSVLTQPPSASGTPGQRVTISCSGSSRNIGAFYVYWYQQLPGTAPKLLIYDNRQRPSGVPDRFSGSKSGTSASLAISGLRSEDEADYYCVAWDDSWSGHWVFGGGTKLTVL

SEQ ID NO: 149 - protein, 119 aa, Homo Sapiens; identical to SEQ ID NO: 131

SEQ ID NO: 150 - protein, 111 aa, Homo Sapiens
QSVLTQPPSASGTPGQRVTISCSGSSRNIGSYYVYWYQQLPGTAPKLLIYDNNQRPSGVPDRFSGSKSGTSASLAISGLRSEDEADYYCVAWDDSWSGTWVFGGGTKLTVL

SEQ ID NO: 151 - protein, 119 aa, Homo Sapiens; identical to SEQ ID NO: 129

SEQ ID NO: 152 - protein, 111 aa, Homo Sapiens
QSVLTQPPSASGTPGQRVTISCSGSSRNIGSFYVYWYQQLPGTAPKLLIYDNRQRPSGVPDRFSGSKSGTSASLAISGLRSEDEADYYCVAWDDSWSGTWVFGGGTKLTVL

SEQ ID NO: 153 - protein, 119 aa, Homo Sapiens; identical to SEQ ID NO: 129

SEQ ID NO: 154 - protein, 111 aa, Homo Sapiens; identical to SEQ ID NO: 136

SEQ ID NO: 155 - protein, 119 aa, Homo Sapiens; identical to SEQ ID NO: 129

SEQ ID NO: 156 - protein, 111 aa, Homo Sapiens
QSVLTQPPSASGTPGQRVTISCSGSSSNIGSYYVYWYQQLPGTAPKLLIYDNNQRPSGVPDRFSGSKSGTSASLAISGLRSEDEADYYCVAWDDSWSGHWVFGGGTKLTVL

SEQ ID NO: 157 - protein, 119 aa, Homo Sapiens; identical to SEQ ID NO: 127

SEQ ID NO: 158 - protein, 111 aa, Homo Sapiens
QSVLTQPPSASGTPGQRVTISCSGSSRNIGSFYVYWYQQLPGTAPKLLIYDNRQRPSGVPDRFSGSKSGTSASLAISGLRSEDEADYYCVAWDDSYSGTWVFGGGTKLTVL

SEQ ID NO: 159 - protein, 119 aa, Homo Sapiens; identical to SEQ ID NO: 129

SEQ ID NO: 160 - protein, 111 aa, Homo Sapiens
QSVLTQPPSASGTPGQRVTISCSGSSRNIGAFYVYWYQQLPGTAPKLLIYDNRQRPSGVPDRFSGSKSGTSASLAISGLRSEDEADYYCVAWDDSWSGTWVFGGGTKLTVL

SEQ ID NO: 161 - protein, 119 aa, Homo Sapiens; identical to SEQ ID NO: 127

SEQ ID NO: 162 - protein, 111 aa, Homo Sapiens; identical to SEQ ID NO: 150

SEQ ID NO: 163 - protein, 119 aa, Homo Sapiens; identical to SEQ ID NO: 131

SEQ ID NO: 164 - protein, 111 aa, Homo Sapiens
QSVLTQPPSASGTPGQRVTISCSGSSRNIGSFYVYWYQQLPGTAPKLLIYDNRQRPSGVPDRFSGSKSGTSASLAISGLRSEDEADYYCVAWDDSLSGHWVFGGGTKLTVL

SEQ ID NO: 165 - protein, 119 aa, Homo Sapiens; identical to SEQ ID NO: 127

SEQ ID NO: 166 - protein, 111 aa, Homo Sapiens
QSVLTQPPSASGTPGQRVTISCSGSSSNIGAYYVYWYQQLPGTAPKLLIYDNNQRPSGVPDRFSGSKSGTSASLAISGLRSEDEADYYCVAWDDSLSGTWVFGGGTKLTVL

SEQ ID NO: 167 - protein, 119 aa, Homo Sapiens
EVQLLESGGGLVQPGGSLRLSCAASGFRFSSYAMSWVRQAPGKGLEWVGGISGSGGSTYYADSVKGRFTISRDNSKNTLYLQMNSLRAEDTAVYYCARQGETSFYLDVWGQGTLVTVSS

SEQ ID NO: 168 - protein, 111 aa, Homo Sapiens
QSVLTQPPSASGTPGQRVTISCSGSSSNIGSYYVYWYHQLPGTAPKLLIYDNNQRPSGVPDRFSGSKSGTSASLAISGLRSEDEADYYCVAWDDSWSGTWVFGGGTKLTVL

SEQ ID NO: 169 - protein, 119 aa, Homo Sapiens; identical to SEQ ID NO: 129

SEQ ID NO: 170 - protein, 111 aa, Homo Sapiens
QSVLTQPPSASGTPGQRVTISCSGSSSNIGAYYVYWYQQLPGTAPKLLIYDNNQRPSGVPDRFSGSKSGTSASLAISGLRSEDEADYYCVAWDDSWSGTWVFGGGTKLTVL

SEQ ID NO: 171 - protein, 119 aa, Homo Sapiens; identical to SEQ ID NO: 127

SEQ ID NO: 172 - protein, 111 aa, Homo Sapiens
QSVLTQPPSASGTPGQRVTISCSGSSRNIGAFYVYWYQQLPGTAPKLLIYDNNQRPSGVPDRFSGSKSGTSASLAISGLRSEDEADYYCVAWDDSLSGTWVFGGGTKLTVL

SEQ ID NO: 173 - protein, 119 aa, Homo Sapiens; identical to SEQ ID NO: 131

SEQ ID NO: 174 - protein, 111 aa, Homo Sapiens
QSVLTQPPSASGTPGQRVTISCSGSSRNIGSYYVYWYQQLPGTAPKLLIYDNNQRPSGVPDRFSGSKSGTSASLAISGLRSEDEADYYCVAWDDSLSGTWVFGGGTKLTVL

SEQ ID NO: 175 - protein, 119 aa, Homo Sapiens; identical to SEQ ID NO: 127

SEQ ID NO: 176 - protein, 111 aa, Homo Sapiens
QSVLTQPPSASGTPGQRVTISCSGSSSNIGAYYVYWYQQLPGTAPKLLIYDNNQRPSGVPDRFSGSKSGTSASLAISGLRSEDEADYYCVAWDDSYSGTWVFGGGTKLTVL

SEQ ID NO: 177 - protein, 119 aa, Homo Sapiens
EVQLLESGGGLVQPGGSLRLSCAASGFTFSSYAMSWVRQAPGKGLEWVGGISGSGGSTYYADSVKGRFTISRDNSKNTLYLQMNSLRAEDTAVYYCARQGKTSFYLDVWGQGTLVTVSS

SEQ ID NO: 178 - protein, 111 aa, Homo Sapiens
QSVLTQPPSASGTPGQRVTISCSGSSSNIGSFYVYWYQQLPGTAPKLLIYDNNQRPSGVPDRFSGSKSGTSASLAISGLRSEDEADYYCVAWDDSYSGTWVFGGGTKLTVL

SEQ ID NO: 179 - protein, 119 aa, Homo Sapiens; identical to SEQ ID NO: 129

SEQ ID NO: 180 - protein, 111 aa, Homo Sapiens
QSVLTQPPSASGTPGQRVTISCSGSSSNIGAYYVYWYQQLPGTAPKLLIYDNNQRPSGVPDRFSGSKSGTSASLAISGLRSEDEADYYCVAWDDSYSGHWVFGGGTKLTVL

SEQ ID NO: 181 - protein, 119 aa, Homo Sapiens; identical to SEQ ID NO: 129

SEQ ID NO: 182 - protein, 111 aa, Homo Sapiens
QSVLTQPPSASGTPGQRVTISCSGSSSNIGSYYVYWYQQLPGTAPKLLIYDNNQRPSGVPDRFSGSKSGTSASLAISGLRSEDEADYYCVAWDDSYSGHWVFGGGTKLTVL

SEQ ID NO: 183 - protein, 119 aa, Homo Sapiens; identical to SEQ ID NO: 135

SEQ ID NO: 184 - protein, 111 aa, Homo Sapiens
QSVLTQPPSASGTPGQRVTISCSGSSSNIGAYYVYWYQQLPGTAPKLLIYDNNQRPSGVPDRFSGSKSGTSASLAISGLRSEDEADYYCVAWDWSWSGTWVFGGGTKLTVL

SEQ ID NO: 185 - protein, 119 aa, Homo Sapiens; identical to SEQ ID NO: 131

SEQ ID NO: 186 - protein, 111 aa, Homo Sapiens
QSVLTQPPSASGTPGQRVTISCSGSSRNIGSFYVYWYQQLPGTAPKLLIYDNNQRPSGVPDRFSGSKSGTSASLAISGLRSEDEADYYCVAWDDSLSGHWVFGGGTKLTVL

SEQ ID NO: 187 - protein, 119 aa, Homo Sapiens; identical to SEQ ID NO: 129

SEQ ID NO: 188 - protein, 111 aa, Homo Sapiens
QSVLTQPPSASGTPGQRVTISCSGSSSNIGAFYVYWYQQLPGTAPKLLIYDNNQRPSGVPDRFSGSKSGTSASLAISGLRSEDEADYYCVAWDWSLSGTWVFGGGTKLTVL

SEQ ID NO: 189 - protein, 119 aa, Homo Sapiens; identical to SEQ ID NO: 127

SEQ ID NO: 190 - protein, 111 aa, Homo Sapiens
QSVLTQPPSASGTPGQRVTISCSGSSSNIGAYYVYWYQQLPGTAPKLLIYDNRQRPSGVPDRFSGSKSGTSASLAISGLRSEDEADYYCVAWDDSLSGHWVFGGGTKLTVL

SEQ ID NO: 191 - protein, 119 aa, Homo Sapiens; identical to SEQ ID NO: 127

SEQ ID NO: 192 - protein, 111 aa, Homo Sapiens
QSVLTQPPSASGTPGQRVTISCSGSSRNIGAFYVYWYQQLPGTAPKLLIYDNNQRPSGVPDRFSGSKSGTSASLAISGLRSEDEADYYCVAWDWSYSGHWVFGGGTKLTVL

SEQ ID NO: 193 - protein, 119 aa, Homo Sapiens; identical to SEQ ID NO: 127

SEQ ID NO: 194 - protein, 111 aa, Homo Sapiens
QSVLTQPPSASGTPGQRVTISCSGSTSNIGSYYVYWYQQLPGTAPKLLIYDNRQRPSGVPDRFSGSKSGTSASLAISGLRSEDEADYYCVAWDDSLSGTWVFGGGTKLTVL

SEQ ID NO: 195 - protein, 119 aa, Homo Sapiens
EVQLLESGGGLVQPGGSLRLSCAASGFTFSSYAMSWVRQAPGKGLEWVSGISGSGGSTYYADSVKGRFTISRDNSKNTLYLQMNSLRAEDTAVYYCAREGETSFYLDVWGQGTLVTVSS

SEQ ID NO: 196 - protein, 111 aa, Homo Sapiens
QSVLTQPPSASGTPGQRVTISCSGSSSNIGSNYVYWYQQLPGTAPKLLIYDNNQRPSGVPDRFSGSKSGTSASLAISGLRSEDEADYYCVAWDDSLSGHWVFGGGTKLTVL

SEQ ID NO: 197 - protein, 119 aa, Homo Sapiens; identical to SEQ ID NO: 195

SEQ ID NO: 198 - protein, 111 aa, Homo Sapiens
QSVLTQPPSASGTPGQRVTISCSGSSSNIGSYYVYWYQQLPGTAPKLLIYDNNQRPSGVPDRFSGSKSGTSASLAISGLRSEDEADYYCVAWDDSLSGHWVFGGGTKLTVL

SEQ ID NO: 199 - protein, 119 aa, Homo Sapiens; identical to SEQ ID NO: 195

SEQ ID NO: 200 - protein, 111 aa, Homo Sapiens; identical to SEQ ID NO: 156

SEQ ID NO: 201 - protein, 119 aa, Homo Sapiens
EVQLLESGGGLVQPGGSLRLSCAASGFTFGDYAMSWVRQAPGKGLEWVSGISGSGGSTYYADSVKGRFTISRDNSKNTLYLQMNSLRAEDTAVYYCAREGETSFGLDVWGQGTLVTVTS

SEQ ID NO: 202 - protein, 111 aa, Homo Sapiens; identical to SEQ ID NO: 196

SEQ ID NO: 203 - protein, 119 aa, Homo Sapiens; identical to SEQ ID NO: 195

SEQ ID NO: 204 - protein, 111 aa, Homo Sapiens
QSVLTQPPSASGTPGQRVTISCSGSSSNIGSNYVYWYQQLPGTAPKLLIYDNNDRPSGVPDRFSGSKSGTSASLAISGLRSEDEADYYCVAWDDSLNGHWVFGGGTKLTVL

SEQ ID NO: 205 - protein, 119 aa, Homo Sapiens
EVQLLESGGGLVQPGGSLRLSCAASGFTFGSYAMSWVRQAPGKGLEWVSGISGSGGSTYYADSVKGRFTISRDNSKNTLYLQMNSLRAEDTAVYYCAREGETSFYLDVWGQGTLVTVSS

SEQ ID NO: 206 - protein, 111 aa, Homo Sapiens; identical to SEQ ID NO: 196

SEQ ID NO: 207 - protein, 119 aa, Homo Sapiens; identical to SEQ ID NO: 201

SEQ ID NO: 208 - protein, 111 aa, Homo Sapiens; identical to SEQ ID NO: 204

SEQ ID NO: 209 - protein, 125 aa, Homo Sapiens
EVQLLESGGGLVQPGGSLRLSCAASGFTFSSYAMSWVRQAPGKGLEWVSGISGSGGGTYYADSVKGRFTISRDNSKNTLYLQMNSLRAEDTAVYYCAREGGTYYYGSGSYVLDYWGQGTLVTVTS

SEQ ID NO: 210 - protein, 110 aa, Homo Sapiens
QSVLTQPPSASGTPGQRVTISCSGSSSNIGSNYVYWYQQLPGTAPKLLIYRNNHRPSGVPDRFSGSKSGTSASLAISGLRSEDEADYYCAAWDDILSGWVFGGGTKLTVL

SEQ ID NO: 211 - protein, 124 aa, Homo Sapiens
EVQLLESGGGLVQPGGSLRLSCAASGFTFSSYAMSWVRQAPGKGLEWVSGISGSGGSTFYADSVKGRFTISRDNSENTLYLQMNSLRAEDTAVYYCARQTSSGLAGYSYGLDPWGQGTLVTVTS

SEQ ID NO: 212 - protein, 111 aa, Homo Sapiens
QSVLTQPPSASGTPGQRVTISCTGSSSNIGAGYDVHWYQQLPGTAPKLLIYSNNQRPSGVPDRFSGSKSGTSASLAISGLRSEDEADYYCAGWDDSLSGWVFGGGTKLTVL

SEQ ID NO: 213 - protein, 117 aa, Homo Sapiens
EVQLLESGGGLVQPGGSLRLSCAASGFTFSAYWMAWVRQAPGKGLEWVSSISSSSSYIYYADSVKGRFTISRDNSKNTLYLQMNSLRAEDTAVYYCARVTEGGLDYWGQGTLVTVTS

SEQ ID NO: 214 - protein, 110 aa, Homo Sapiens
QSVLTQPPSASGTPGQRVTISCSGSSSNIGNNAVNWYQQLPGTAPKLLIYRNNQRPSGVPDRFSGSKSGTSASLAISGLRSEDEADYYCQSYDSSLSGWVFGGGTKLTVL

SEQ ID NO: 215 - protein, 118 aa, Homo Sapiens
EVQLLESGGGLVQPGGSLRLSCAASGFTFSSYGMSWVRQAPGKGLEWVSGISGSGGSTYYADSVKGRFTISRDNSKNTLYLQMNSLRAEDTAVYYCARDGGYDELDYWGQGTLVTVTS

SEQ ID NO: 216 - protein, 111 aa, Homo Sapiens
QSVLTQPPSASGTPGQRVTISCTGSSSNIGAGYVVHWYQQLPGTAPKLLIYDNNKRPSGVPDRFSGSKSGTSASLAISGLRSEDEADYYCAAWDDSLSAWVFGGGTKLTVL

SEQ ID NO: 217 - protein, 117 aa, Homo Sapiens; identical to SEQ ID NO: 125

SEQ ID NO: 218 - protein, 110 aa, Homo Sapiens; identical to SEQ ID NO: 124

SEQ ID NO: 219 - protein, 117 aa, Homo Sapiens
EVQLLESGGGLVQPGGSLRLSCAASGFTFSNAWMSWVRQAPGKGLEWVSSIHWNGGYIGYAESVKGRFTISRDNSKNTLYLQMNSLRAEDTAVYYCARVTLDAVDYWGQGTLVTVTS

SEQ ID NO: 220 - protein, 110 aa, Homo Sapiens
QSVLTQPPSASGTPGQRVTISCSGSSSNIGNNAVNWYQQLPGTAPKLLIYRNNQRPSGVPDRFSGSKSGTSASLAISGLRSEDEADYYCQSYDNRLTAWVFGGGTKLTVL

SEQ ID NO: 221 - protein, 5 aa, Homo Sapiens
DYAMS

SEQ ID NO: 222 - protein, 17 aa, Homo Sapiens
GISGSGGSTYYADSVKG

SEQ ID NO: 223 - protein, 10 aa, Homo Sapiens
EGETSFGLDV

SEQ ID NO: 224 - protein, 13 aa, Homo Sapiens
SGSSSNIGSNYVY

SEQ ID NO: 225 - protein, 7 aa, Homo Sapiens
DNNDRPS

SEQ ID NO: 226 - protein, 12 aa, Homo Sapiens
VAWDDSLNGHWV

SEQ ID NO: 227 - protein, 5 aa, Homo Sapiens
SYAMS

SEQ ID NO: 228 - protein, 17 aa, Homo Sapiens
GISGSGGGTYYADSVKG

SEQ ID NO: 229 - protein, 16 aa, Homo Sapiens
EGGTYYYGSGSYVLDY

SEQ ID NO: 230 - protein, 13 aa, Homo Sapiens
SGSSSNIGSNYVY

SEQ ID NO: 231 - protein, 7 aa, Homo Sapiens
RNNHRPS

SEQ ID NO: 232 - protein, 11 aa, Homo Sapiens
AAWDDILSGWV

SEQ ID NO: 233 - protein, 5 aa, Homo Sapiens
SYAMS

SEQ ID NO: 234 - protein, 17 aa, Homo Sapiens
GISGSGGSTFYADSVKG

SEQ ID NO: 235 - protein, 15 aa, Homo Sapiens
QTSSGLAGYSYGLDP

SEQ ID NO: 236 - protein, 14 aa, Homo Sapiens
TGSSSNIGAGYDVH

SEQ ID NO: 237 - protein, 7 aa, Homo Sapiens
SNNQRPS

SEQ ID NO: 238 - protein, 11 aa, Homo Sapiens
AGWDDSLSGWV

SEQ ID NO: 239 - protein, 5 aa, Homo Sapiens
AYWMA

SEQ ID NO: 240 - protein, 10 aa, Homo Sapiens
SISSSSSYIY

SEQ ID NO: 241 - protein, 8 aa, Homo Sapiens
VTEGGLDY

SEQ ID NO: 242 - protein, 13 aa, Homo Sapiens
SGSSSNIGNNAVN

SEQ ID NO: 243 - protein, 7 aa, Homo Sapiens
RNNQRPS

SEQ ID NO: 244 - protein, 11 aa, Homo Sapiens
QSYDSSLSGWV

SEQ ID NO: 245 - protein, 5 aa, Homo Sapiens
SYGMS

SEQ ID NO: 246 - protein, 17 aa, Homo Sapiens
GISGSGGSTYYADSVKG

SEQ ID NO: 247 - protein, 9 aa, Homo Sapiens
DGGYDELDY

SEQ ID NO: 248 - protein, 14 aa, Homo Sapiens
TGSSSNIGAGYVVH

SEQ ID NO: 249 - protein, 7 aa, Homo Sapiens
DNNKRPS

SEQ ID NO: 250 - protein, 11 aa, Homo Sapiens
AAWDDSLSAWV

SEQ ID NO: 251 - protein, 5 aa, Homo Sapiens
NAWMS

SEQ ID NO: 252 - protein, 17 aa, Homo Sapiens
SISSSSGYIYYADSLKG

SEQ ID NO: 253 - protein, 8 aa, Homo Sapiens
VWRNHLDY

SEQ ID NO: 254 - protein, 13 aa, Homo Sapiens
SGSSSDIGSNTVN

SEQ ID NO: 255 - protein, 7 aa, Homo Sapiens
DNNQRPS

SEQ ID NO: 256 - protein, 11 aa, Homo Sapiens
QSYDSSLSGWV

SEQ ID NO: 257 - protein, 5 aa, Homo Sapiens
NAWMS

SEQ ID NO: 258 - protein, 17 aa, Homo Sapiens
SIHWNGGYIGYAESVKG

SEQ ID NO: 259 - protein, 8 aa, Homo Sapiens
VTLDAVDY

SEQ ID NO: 260 - protein, 13 aa, Homo Sapiens
SGSSSNIGNNAVN

SEQ ID NO: 261 - protein, 7 aa, Homo Sapiens
RNNQRPS

SEQ ID NO: 262 - protein, 11 aa, Homo Sapiens
QSYDNRLTAWV

SEQ ID NO: 263 - protein, 5 aa, Homo Sapiens
SYWMS

SEQ ID NO: 264 - protein, 17 aa, Homo Sapiens
SISSSSSYIYYADSVKG

SEQ ID NO: 265 - protein, 8 aa, Homo Sapiens
VWRNYLDY

SEQ ID NO: 266 - protein, 13 aa, Homo Sapiens
SGSSSNIGSNTVN

SEQ ID NO: 267 - protein, 7 aa, Homo Sapiens
SNNQRPS

SEQ ID NO: 268 - protein, 11 aa, Homo Sapiens
QSYDSSLSGWV

SEQ ID NO: 269 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 270 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 271 - DNA, 351 bp, Homo Sapiens; identical to SEQ ID NO: 269

SEQ ID NO: 272 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 273 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 274 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 275 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 276 - DNA, 330 bp, Homo Sapiens; identical to SEQ ID NO: 274

SEQ ID NO: 277 - DNA, 351 bp, Homo Sapiens; identical to SEQ ID NO: 273

SEQ ID NO: 278 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 279 - DNA, 351 bp, Homo Sapiens; identical to SEQ ID NO: 275

SEQ ID NO: 280 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 281 - DNA, 351 bp, Homo Sapiens; identical to SEQ ID NO: 273

SEQ ID NO: 282 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 283 - DNA, 351 bp, Homo Sapiens; identical to SEQ ID NO: 275

SEQ ID NO: 284 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 285 - DNA, 351 bp, Homo Sapiens; identical to SEQ ID NO: 269

SEQ ID NO: 286 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcacctcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 287 - DNA, 351 bp, Homo Sapiens
gaagttcagctgctggaaagcggtggtggtctggttcagcctggtggtagcctgcgtctgagctgcgctgcaagcggttttacctttagcgatagctggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacagcctggattattggggtcagggcaccctggttaccgttagcagc

SEQ ID NO: 288 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaactgctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 289 - DNA, 351 bp, Homo Sapiens; identical to SEQ ID NO: 287

SEQ ID NO: 290 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaactgctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 291 - DNA, 351 bp, Homo Sapiens; identical to SEQ ID NO: 275

SEQ ID NO: 292 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaagttctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 293 - DNA, 351 bp, Homo Sapiens; identical to SEQ ID NO: 273

SEQ ID NO: 294 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 295 - DNA, 351 bp, Homo Sapiens; identical to SEQ ID NO: 273

SEQ ID NO: 296 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttggagcaataatcagcatccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 297 - DNA, 351 bp, Homo Sapiens
gaagttcagctgctggaaagcagtggtggtctggttcagcctggtggtagcctgcgtctgagctgcgctgcaagcggttttacctttagcgattattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacagcctgagctattggggtcagggcaccctggttaccgttagcagc

SEQ ID NO: 298 - DNA, 330 bp, Homo Sapiens; identical to SEQ ID NO: 294

SEQ ID NO: 299 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatggcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 300 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 301 - DNA, 351 bp, Homo Sapiens; identical to SEQ ID NO: 273

SEQ ID NO: 302 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 303 - DNA, 351 bp, Homo Sapiens; identical to SEQ ID NO: 275

SEQ ID NO: 304 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 305 - DNA, 351 bp, Homo Sapiens; identical to SEQ ID NO: 273

SEQ ID NO: 306 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttggagcaataatcagcgtccgaacggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 307 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcagcagcagctcttatatttattatgccgatagcgtgaaaggtcgctttaccattagccgtgataatagcaaaaacacactgtatctgcagatgaatagcctgcgtgcagaagataccgcagtttattattgtgcacgtgtgtggcgtaactatctggattattggggtcagggcaccctggttaccgttagcagc

SEQ ID NO: 308 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 309 - DNA, 351 bp, Homo Sapiens; identical to SEQ ID NO: 275

SEQ ID NO: 310 - DNA, 330 bp, Homo Sapiens; identical to SEQ ID NO: 278

SEQ ID NO: 311 - DNA, 351 bp, Homo Sapiens; identical to SEQ ID NO: 269

SEQ ID NO: 312 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaaagcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 313 - DNA, 351 bp, Homo Sapiens; identical to SEQ ID NO: 269

SEQ ID NO: 314 - DNA, 330 bp, Homo Sapiens; identical to SEQ ID NO: 312

SEQ ID NO: 315 - DNA, 351 bp, Homo Sapiens; identical to SEQ ID NO: 273

SEQ ID NO: 316 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 317 - DNA, 351 bp, Homo Sapiens; identical to SEQ ID NO: 275

SEQ ID NO: 318 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 319 - DNA, 351 bp, Homo Sapiens; identical to SEQ ID NO: 273

SEQ ID NO: 320 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 321 - DNA, 351 bp, Homo Sapiens; identical to SEQ ID NO: 269

SEQ ID NO: 322 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 323 - DNA, 351 bp, Homo Sapiens; identical to SEQ ID NO: 273

SEQ ID NO: 324 - DNA, 330 bp, Homo Sapiens
cagagcgttctgacccagcctccgagcacaagcggtacaccgggtcagcgtgttaccattagctgtagcggtagcagcagcaatattggtagcaataccgttaattggtatcagcagctgcctggcaccgcaccgaaagttctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 325 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacgcactggattattggggtcagggcaccctggttaccgttagcagc

SEQ ID NO: 326 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaactgctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 327 - DNA, 351 bp, Homo Sapiens
gaagttcagctgctggaaagcggtggtggtctggttcagcctggtggtagcctgcgtctgagctgcgctgcaagcggttttacctttagcgatagctggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacgcactggattattggggtcagggcaccctggttaccgttagcagc

SEQ ID NO: 328 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaactgctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 329 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcaaggcaccctggttaccgttagcagc

SEQ ID NO: 330 - DNA, 330 bp, Homo Sapiens; identical to SEQ ID NO: 308

SEQ ID NO: 331 - DNA, 351 bp, Homo Sapiens; identical to SEQ ID NO: 275

SEQ ID NO: 332 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttggagcaataatcagcgtccgagcagtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 333 - DNA, 351 bp, Homo Sapiens
gaagttcagctgctggaaagcggtggtggtctggttcagactggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 334 - DNA, 330 bp, Homo Sapiens; identical to SEQ ID NO: 320

SEQ ID NO: 335 - DNA, 351 bp, Homo Sapiens; identical to SEQ ID NO: 275

SEQ ID NO: 336 - DNA, 330 bp, Homo Sapiens; identical to SEQ ID NO: 282

SEQ ID NO: 337 - DNA, 351 bp, Homo Sapiens; identical to SEQ ID NO: 275

SEQ ID NO: 338 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 339 - DNA, 351 bp, Homo Sapiens
gaagttcagctgctggaaagcggtggtggtctggttcagcctggtggtagcctgcgtctgagctgcgctgcaagcggttttacctttagcgatagctggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 340 - DNA, 330 bp, Homo Sapiens; identical to SEQ ID NO: 284

SEQ ID NO: 341 - DNA, 351 bp, Homo Sapiens; identical to SEQ ID NO: 275

SEQ ID NO: 342 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaagttctgatttatagcaataatcagcgtccgaacggtgttccggatcgttttagcggtagcaaaagcggcaccagcgcaagcctggcaattaccggtctgcaggcagaagatgaagcagattattattgccagagctatagtagcagcctgagcggttgggtttttggtggtggcaccaaactgaccgttctg

SEQ ID NO: 343 - DNA, 351 bp, Homo Sapiens; identical to SEQ ID NO: 275

SEQ ID NO: 344 - DNA, 330 bp, Homo Sapiens; identical to SEQ ID NO: 300

SEQ ID NO: 345 - DNA, 351 bp, Homo Sapiens
gaagttcagctgctggaaagcggtggtggtctggttcagcctggtggtagcctgcgtctgagctgcgctgcaagcggttttacctttagcgatcattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 346 - DNA, 330 bp, Homo Sapiens; identical to SEQ ID NO: 272

SEQ ID NO: 347 - DNA, 351 bp, Homo Sapiens; identical to SEQ ID NO: 273

SEQ ID NO: 348 - DNA, 330 bp, Homo Sapiens; identical to SEQ ID NO: 322

SEQ ID NO: 349 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgggataatagcaaaaacaccctgtatctgcagatgaatagcctgcgtgcagaagataccgcagtttattattgtgcacgtgtgtggcgtaacgcactgagctattggggtcagggcaccctggttaccgttagcagc

SEQ ID NO: 350 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaactgctgatttggagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 351 - DNA, 351 bp, Homo Sapiens; identical to SEQ ID NO: 275

SEQ ID NO: 352 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 353 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 354 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaagttctgatttggagcaataatcagcgtccaagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 355 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacgcactgagctattggggtcagggcaccctggttaccgttagcagc

SEQ ID NO: 356 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaaatcctgatttggagcaataatcagcgtccgagcggtgttccggatcgttttagcggtagcaaaagcggcaccagcgcaagcctggcaattaccggtctgcaggcagaagatgaagcagattattattgccagagctatagtagcagcctggttggttgggtttttggtggtggcaccaaactgaccgttctg

SEQ ID NO: 357 - DNA, 351 bp, Homo Sapiens; identical to SEQ ID NO: 275

SEQ ID NO: 358 - DNA, 330 bp, Homo Sapiens
cagagcgttctgacccagcctccgagcgcaagcggtacaccgggtcagcgtgttaccattagctgtagcggtagcagcagcaatattggtagcaataaagttaattggtatcagcagctgcctggcaccgcaccgaaaagcctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 359 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagcagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacagcctgagctattggggtcagggcaccctggttaccgttagcagc

SEQ ID NO: 360 - DNA, 330 bp, Homo Sapiens; identical to SEQ ID NO: 280

SEQ ID NO: 361 - DNA, 351 bp, Homo Sapiens; identical to SEQ ID NO: 275

SEQ ID NO: 362 - DNA, 330 bp, Homo Sapiens; identical to SEQ ID NO: 308

SEQ ID NO: 363 - DNA, 351 bp, Homo Sapiens; identical to SEQ ID NO: 269

SEQ ID NO: 364 - DNA, 330 bp, Homo Sapiens; identical to SEQ ID NO: 302

SEQ ID NO: 365 - DNA, 351 bp, Homo Sapiens
gaagttcagctgctggaaagcggtggtggtctggttcagcctggtggtagcctgcgtctgagctgcgctgcaagcggttttacctttagcgattattggatgagctgggttcgtcaggcaccgggtaaaggtctggaatgggtgagtagcattagcaccagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctgagctattggggtcagggcaccctggttaccgttagcagc

SEQ ID NO: 366 - DNA, 330 bp, Homo Sapiens; identical to SEQ ID NO: 270

SEQ ID NO: 367 - DNA, 351 bp, Homo Sapiens; identical to SEQ ID NO: 269

SEQ ID NO: 368 - DNA, 330 bp, Homo Sapiens; identical to SEQ ID NO: 308

SEQ ID NO: 369 - DNA, 351 bp, Homo Sapiens; identical to SEQ ID NO: 275

SEQ ID NO: 370 - DNA, 330 bp, Homo Sapiens; identical to SEQ ID NO: 338

SEQ ID NO: 371 - DNA, 351 bp, Homo Sapiens
gaagttcagctgctggaaagcggtggtggtctggttcagcctggtggtagcctgcgtctgagctgcgctgcaagcggttttacctttagcagctattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacagcctggattattggggtcagggcaccctggttaccgttagcagc

SEQ ID NO: 372 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaactgctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctggttggttgggtttttggtggtggcaccaaactgaccgttctg

SEQ ID NO: 373 - DNA, 351 bp, Homo Sapiens; identical to SEQ ID NO: 371

SEQ ID NO: 374 - DNA, 330 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaactgctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 375 - DNA, 351 bp, Homo Sapiens
gaagttcagctgctggaaagcggtggtggtctggttcagcctggtggtagcctgcgtctgagctgcgctgcaagcggttttacctttagcagctattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 376 - DNA, 330 bp, Homo Sapiens; identical to SEQ ID NO: 372

SEQ ID NO: 377 - DNA, 353 bp, Homo Sapiens
cagaagttcagctgctggaaagcggtggtggtctggttcagcctggtggtagcctgcgtctgagctgcgctgcaagcggttttacctttagcaattattggatgagctgggttcgtcaggcaccgggtaaaggtctggaatgggtgagcagcattagcagcagcagcggttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 378 - DNA, 330 bp, Homo Sapiens; identical to SEQ ID NO: 374

SEQ ID NO: 379 - DNA, 351 bp, Homo Sapiens
gaagttcagctgctggaaagcggtggtggtctggttcagcctggtggtagcctgcgtctgagctgcgctgcaagcggttttacctttagcaattattggatgagctgggttcgtcaggcaccgggtaaaggtctggaatgggtgagcagcattagcagcagcagcggttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagc

SEQ ID NO: 380 - DNA, 330 bp, Homo Sapiens; identical to SEQ ID NO: 372

SEQ ID NO: 381 - DNA, 351 bp, Homo Sapiens
gaagttcagctgctggaaagcggtggtggtctggttcagcctggtggtagcctgcgtctgagctgcgctgcaagcggttttacctttagcaattattggatgagctgggttcgtcaggcaccgggtaaaggtctggaatgggtgagcagcattagcagcagcagcggttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaacagcctggattattggggtcagggcaccctggttaccgttagcagc

SEQ ID NO: 382 - DNA, 330 bp, Homo Sapiens; identical to SEQ ID NO: 372

SEQ ID NO: 383 - DNA, 351 bp, Homo Sapiens; identical to SEQ ID NO: 381

SEQ ID NO: 384 - DNA, 330 bp, Homo Sapiens; identical to SEQ ID NO: 374

SEQ ID NO: 385 - DNA, 351 bp, Homo Sapiens; identical to SEQ ID NO: 375

SEQ ID NO: 386 - DNA, 330 bp, Homo Sapiens; identical to SEQ ID NO: 374

SEQ ID NO: 387 - DNA, 351 bp, Homo Sapiens; identical to SEQ ID NO: 379

SEQ ID NO: 388 - DNA, 330 bp, Homo Sapiens
cagagcgttctgacccagcctccgagcgcaagcggtacaccgggtcagcgtgttaccattagctgtagcggtagcagcagcgatattggtagcaataccgttaattggtatcagcagctgcctggcaccgcaccgaaactgctgatttatgataataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctg

SEQ ID NO: 389 - DNA, 351 bp, Homo Sapiens; identical to SEQ ID NO: 375

SEQ ID NO: 390 - DNA, 330 bp, Homo Sapiens; identical to SEQ ID NO: 388

SEQ ID NO: 391 - DNA, 351 bp, Homo Sapiens; identical to SEQ ID NO: 375

SEQ ID NO: 392 - DNA, 330 bp, Homo Sapiens
cagagcgttctgacccagcctccgagcgcaagcggtacaccgggtcagcgtgttaccattagctgttctggtagcagcagcgatattggtagcaataccgtgaattggtatcagcagctgcctggtacagcaccgaaactgctgatttatgataataatcagcgtcctagcggtgttccggatcgttttagcggtagcaaaagcggcaccagcgcaagcctggcaattagcggtctgcgtagcgaagatgaagcagattattattgtcagagctatgatagcagcctgagcggttgggtttttggtggtggcaccaaactgaccgttctg

SEQ ID NO: 393 - DNA, 351 bp, Homo Sapiens
gaagttcagctgctggaaagcggtggtggtctggttcagcctggtggtagcctgcgtctgagctgtgcagcaagcggttttacctttagcaatgcatggatgagctgggttcgtcaggcaccgggtaaaggtctggaatgggtgagcagcattagcagcagcagcggttatatttattatgcagatagcctgaaaggtcgctttaccattagccgtgataatagcaaaaataccctgtatctgcagatgaatagcctgcgtgcagaagataccgcagtttattattgcgcacgtgtttggcgtaatcatctggattattggggtcagggcaccctggttaccgttaccagc

SEQ ID NO: 394 - DNA, 330 bp, Homo Sapiens; identical to SEQ ID NO: 374

SEQ ID NO: 395 - DNA, 357 bp, Homo Sapiens
gaagttcagctgctggaaagcggtggtggtctggttcagcctggtggtagcctgcgtctgagctgcgctgcaagcggttttcgttttagcagctatgcaatgagctgggttcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 396 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcaacagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 397 - DNA, 357 bp, Homo Sapiens
gaagttcagctgctggaaagcggtggtggtctggttcagcctggtggtagcctgcgtctgagctgcgctgcaagcggttttacctttagcagctatgcaatgagctgggttcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 398 - DNA, 333 bp, Homo Sapiens
cagagcgttctgacccagcctccgagcgcaagcggtacaccgggtcagcgtgttaccattagctgttgcggtagcagccgtaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 399 - DNA, 357 bp, Homo Sapiens
gaagttcagctgctggaaagcggtggtggtctggttcagcctggtggtagcctgcgtctgagctgcgctgcaagcggttttacctttagcagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 400 - DNA, 333 bp, Homo Sapiens
cagagcgttctgacccagcctccgagcgcaagcggtacaccgggtcagcgtgttaccattagctgtagcggtagcagccgtaatattggtagctattatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 401 - DNA, 357 bp, Homo Sapiens
gaagttcagctgctggaaagcggtggtggtctggttcagcctggtggtagcctgcgtctgagctgcgctgcaagcggttttcgttttagcagctatgcaatgagctgggttcgtcaggcaccgggtaaaggtcttgaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 402 - DNA, 333 bp, Homo Sapiens
cagagcgttctgacccagcctccgagcgcaagcggtacaccgggtcagcgtgttaccattagctgtagcggtagcagccgtaatattggtgcatattatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 403 - DNA, 359 bp, Homo Sapiens
cagaagttcagctgctggaaagcggtggtggtctggttcagcctggtggtagcctgcgtctgagctgcgctgcaagcggttttcgttttagcagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 404 - DNA, 333 bp, Homo Sapiens
cagagcgttctgacccagcctccgagcgcaagcggtacaccgggtcagcgtgttaccattagctgtagcggtagcagccgtaatattggtagctattatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 405 - DNA, 357 bp, Homo Sapiens
gaagttcagctgctggaaagcggtggtggtctggttcagcctggtggtagcctgcgtctgagctgcgctgcaagcggttttcgttttagcagctatgcaatgagctggattcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 406 - DNA, 333 bp, Homo Sapiens; identical to SEQ ID NO: 404

SEQ ID NO: 407 - DNA, 357 bp, Homo Sapiens; identical to SEQ ID NO: 395

SEQ ID NO: 408 - DNA, 333 bp, Homo Sapiens
cagagcgttctgacccagcctccgagcgcaagcggtacaccgggtcagcgtgttaccattagctgtagcggtagcagccgtaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtcattgggtttttggtggtggcaccaaactgaccgttctg

SEQ ID NO: 409 - DNA, 357 bp, Homo Sapiens; identical to SEQ ID NO: 397

SEQ ID NO: 410 - DNA, 333 bp, Homo Sapiens
cagagcgttctgacccagcctccgagcgcaagcggtacaccgggtcagcgtgttaccattagctgtagcggtagcagccgtaatattggtgcattttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 411 - DNA, 357 bp, Homo Sapiens; identical to SEQ ID NO: 395

SEQ ID NO: 412 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgttagcctgagcggtcattgggtttttggtggtggcaccaaactgaccgttctg

SEQ ID NO: 413 - DNA, 357 bp, Homo Sapiens; identical to SEQ ID NO: 397

SEQ ID NO: 414 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtcattgggtttttggtggtggcaccaaactgaccgttctg

SEQ ID NO: 415 - DNA, 357 bp, Homo Sapiens; identical to SEQ ID NO: 395

SEQ ID NO: 416 - DNA, 333 bp, Homo Sapiens
cagagcgttctgacccagcctccgagcgcaagcggtacaccgggtcagcgtgttaccattagctgtagcggtagcagccgaaatattggtgcattttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctggagcggtcattgggtttttggtggtggcaccaaactgaccgttctg

SEQ ID NO: 417 - DNA, 357 bp, Homo Sapiens; identical to SEQ ID NO: 399

SEQ ID NO: 418 - DNA, 333 bp, Homo Sapiens
cagagcgttctgacccagcctccgagcgcaagcggtacaccgggtcagcgtgttaccattagctgtagcggtagcagccgtaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 419 - DNA, 357 bp, Homo Sapiens; identical to SEQ ID NO: 397

SEQ ID NO: 420 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 421 - DNA, 357 bp, Homo Sapiens; identical to SEQ ID NO: 397

SEQ ID NO: 422 - DNA, 333 bp, Homo Sapiens; identical to SEQ ID NO: 404

SEQ ID NO: 423 - DNA, 357 bp, Homo Sapiens; identical to SEQ ID NO: 397

SEQ ID NO: 424 - DNA, 333 bp, Homo Sapiens
cagagcgttctgacccagcctccgagcgcaagcggtacaccgggtcagcgtgttaccattagctgtagcggtagcagcagcaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtcattgggtttttggtggtggcaccaaactgaccgttctg

SEQ ID NO: 425 - DNA, 357 bp, Homo Sapiens; identical to SEQ ID NO: 395

SEQ ID NO: 426 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 427 - DNA, 357 bp, Homo Sapiens; identical to SEQ ID NO: 397

SEQ ID NO: 428 - DNA, 333 bp, Homo Sapiens
cagagcgttctgacccagcctccgagcgcaagcggtacaccgggtcagcgtgttaccattagctgtagcggtagcagccgtaatattggtgcattttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 429 - DNA, 357 bp, Homo Sapiens; identical to SEQ ID NO: 395

SEQ ID NO: 430 - DNA, 333 bp, Homo Sapiens; identical to SEQ ID NO: 418

SEQ ID NO: 431 - DNA, 357 bp, Homo Sapiens; identical to SEQ ID NO: 399

SEQ ID NO: 432 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtcattgggtttttggtggtggcaccaaactgaccgttctg

SEQ ID NO: 433 - DNA, 357 bp, Homo Sapiens; identical to SEQ ID NO: 395

SEQ ID NO: 434 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtacctgggtttttggtggtggcaccaaactgaccgttctg

SEQ ID NO: 435 - DNA, 357 bp, Homo Sapiens
gaagttcagctgctggaaagcggtggtggtctggttcagcctggtggtagcctgcgtctgagctgcgctgcaagcggttttcgttttagcagctatgcaatgagctgggttcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtgaaaccagcttttatctggatgtttggggtcagggcaccctggttaccgttagcagc

SEQ ID NO: 436 - DNA, 333 bp, Homo Sapiens
cagagcgttctgacccagcctccgagcgcaagcggtacaccgggtcagcgtgttaccattagctgtagcggtagcagcagcaatattggtagctattatgtgtactggtatcatcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 437 - DNA, 357 bp, Homo Sapiens; identical to SEQ ID NO: 397

SEQ ID NO: 438 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctggagcggtacctgggtttttggtggtggcaccaaactgaccgttctg

SEQ ID NO: 439 - DNA, 357 bp, Homo Sapiens; identical to SEQ ID NO: 395

SEQ ID NO: 440 - DNA, 333 bp, Homo Sapiens
cagagcgttctgacccagcctccgagcgcaagcggtacaccgggtcagcgtgttaccattagctgtagcggtagcagccgtaatattggtgcattttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtacctgggtttttggtggtggcaccaaactgaccgttctg

SEQ ID NO: 441 - DNA, 357 bp, Homo Sapiens
gaagttcagctgctggaaagcggtggtggtctagttcagcctggtggtagcctgcgtctgagctgcgctgcaagcggttttacctttagcagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 442 - DNA, 333 bp, Homo Sapiens
cagagcgttctgacccagcctccgagcgcaagcggtacaccgggtcagcgtgttaccattagctgtagcggtagcagccgtaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtacctgggtttttggtggtggcaccaaactgaccgttctg

SEQ ID NO: 443 - DNA, 357 bp, Homo Sapiens; identical to SEQ ID NO: 395

SEQ ID NO: 444 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 445 - DNA, 357 bp, Homo Sapiens
gaagttcagctgctggaaagcggtggtggtctggttcagcctggtggtagcctgcgtctgagctgcgctgcaagcggttttacctttagcagctatgcaatgagctgggttcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtaaaaccagcttttatctggatgtttggggtcagggcaccctggttaccgttagcagc

SEQ ID NO: 446 - DNA, 333 bp, Homo Sapiens
cagagcgttctgacccagcctccgagcgcaagcggtacaccgggtcagcgtgttaccattagctgtagcggtagcagcagcaatattggtagcttttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctatagcggtacctgggtttttggtggtggcaccaaactgaccgttctg

SEQ ID NO: 447 - DNA, 357 bp, Homo Sapiens; identical to SEQ ID NO: 397

SEQ ID NO: 448 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctatagcggtcattgggtttttggtggtggcaccaaactgaccgttctg

SEQ ID NO: 449 - DNA, 357 bp, Homo Sapiens; identical to SEQ ID NO: 397

SEQ ID NO: 450 - DNA, 333 bp, Homo Sapiens
cagagcgttctgacccagcctccgagcgcaagcggtacaccgggtcagcgtgttaccattagctgtagcggtagcagcagcaatattggtagctattatgtgtactggtatcaacagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagctatagcggtcattgggtttttggtggtggcaccaaactgaccgttctg

SEQ ID NO: 451 - DNA, 357 bp, Homo Sapiens
gaagttcagctgctggaaagcggtggtggtctggttcagcctggtggtagcctgcgtctgagctgcgctgcaagcggttttcgttttagcagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 452 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggattggagctggagcggtacctgggtttttggtggtggcaccaaactgaccgttctg

SEQ ID NO: 453 - DNA, 357 bp, Homo Sapiens; identical to SEQ ID NO: 399

SEQ ID NO: 454 - DNA, 333 bp, Homo Sapiens
cagagcgttctgacccagcctccgagcgcaagcggtacaccgggtcagcgtgttaccattagctgtagcggtagcagccgtaatattggtagcttttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtcattgggtttttggtggtggcaccaaactgaccgttctg

SEQ ID NO: 455 - DNA, 357 bp, Homo Sapiens; identical to SEQ ID NO: 397

SEQ ID NO: 456 - DNA, 333 bp, Homo Sapiens
cagagcgttctgacccagcctccgagcgcaagcggtacaccgggtcagcgtgttaccattagctgtagcggtagcagcagcaatattggtgcattttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggattggagcctgagcggtacctgggtttttggtggtggcaccaaactgaccgttctg

SEQ ID NO: 457 - DNA, 357 bp, Homo Sapiens
gaagttcagctgctggaaagcggtggtggtctggttcaacctggtggtagcctgcgtctgagctgcgctgcaagcggttttcgttttagcagctatgcaatgagctgggttcgtcaggcaccgggtaaaggtctggaatgggtgggtggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtcagggtcgtaccagcttttatctggatgtttggggtcagggcaccctggttaccgttagcagc

SEQ ID NO: 458 - DNA, 333 bp, Homo Sapiens
cagagcgttctgacccagcctccgagcgcaagcggtacaccgggtcagcgtgttaccattagctgtagcggtagcagcagcaatattggtgcatattatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtcattgggtttttggtggtggcaccaaactgaccgttctg

SEQ ID NO: 459 - DNA, 357 bp, Homo Sapiens; identical to SEQ ID NO: 395

SEQ ID NO: 460 - DNA, 333 bp, Homo Sapiens
cagagcgttctgacccagcctccgagcgcaagcggtacaccgggtcagcgtgttaccattagctgtagcggtagcagccgtaatattggtgcattttatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggattggagctatagcggtcattgggtttttggtggtggcaccaaactgaccgttctg

SEQ ID NO: 461 - DNA, 357 bp, Homo Sapiens; identical to SEQ ID NO: 395

SEQ ID NO: 462 - DNA, 333 bp, Homo Sapiens
cagagcgttctgacccagcctccgagcgcaagcggtacaccgggtcagcgtgttaccattagctgtagcggtagcaccagcaatattggtagctattatgtgtactggtatcagcagctgcctggcaccgcaccgaaactgctgatttatgataatcgtcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtacctgggtttttggtggtggcaccaaactgaccgttctg

SEQ ID NO: 463 - DNA, 357 bp, Homo Sapiens
gaagttcagctgctggaaagcggtggtggtctggttcagcctggtggtagcctgcgtctgagctgcgctgcaagcggttttacctttagcagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtgaaggtgaaaccagcttttatctggatgtttggggtcagggcaccctggttaccgttagcagc

SEQ ID NO: 464 - DNA, 333 bp, Homo Sapiens
cagagcgttctgacccagcctccgagcgcaagcggtacaccgggtcagcgtgttaccattagctgtagcggtagcagcagcaatattggtagcaattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtcattgggtttttggtggtggcaccaaactgaccgttctg

SEQ ID NO: 465 - DNA, 357 bp, Homo Sapiens; identical to SEQ ID NO: 463

SEQ ID NO: 466 - DNA, 333 bp, Homo Sapiens
cagagcgttctgacccagcctccgagcgcaagcggtacaccgggtcagcgtgttaccattagctgtagcggtagcagcagcaatattggtagctattatgtgtactggtatcagcagctgcctggcaccgcaccgaaactgctgatttatgataataatcagcgtccgagcggtgttccggatcgttttagcggtagcaaaagcggcaccagcgcaagcctggcaattagcggtctgcgtagcgaagatgaagcagattattattgtgttgcctgggatgatagcctgagcggtcattgggtttttggtggtggcaccaaactgaccgttctg

SEQ ID NO: 467 - DNA, 357 bp, Homo Sapiens; identical to SEQ ID NO: 463

SEQ ID NO: 468 - DNA, 333 bp, Homo Sapiens; identical to SEQ ID NO: 424

SEQ ID NO: 469 - DNA, 357 bp, Homo Sapiens
gaagttcagctgctggaaagcggtggtggtctggttcagcctggtggtagcctgcgtctgagctgtgcagcaagcggttttacctttggtgattatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaataccctgtatctgcagatgaatagcctgcgtgcagaagataccgcagtttattattgcgcacgtgaaggtgaaaccagctttggtctggatgtttggggtcagggcaccctggttaccgttaccagc

SEQ ID NO: 470 - DNA, 333 bp, Homo Sapiens; identical to SEQ ID NO: 464

SEQ ID NO: 471 - DNA, 357 bp, Homo Sapiens; identical to SEQ ID NO: 463

SEQ ID NO: 472 - DNA, 333 bp, Homo Sapiens
cagagcgttctgacccagcctccgagcgcaagcggtacaccgggtcagcgtgttaccattagctgttctggtagcagcagcaatattggtagcaattatgtgtattggtatcagcagctgcctggtacagcaccgaaactgctgatttatgataataatgatcgccctagcggtgttccggatcgttttagcggtagcaaaagcggcaccagcgcaagcctggcaattagcggtctgcgtagcgaagatgaagcagattattattgtgttgcatgggatgatagcctgaatggtcattgggtttttggtggtggcaccaaactgaccgttctg

SEQ ID NO: 473 - DNA, 357 bp, Homo Sapiens
gaagttcagctgctggaaagcggtggtggtctggttcagcctggtggtagcctgcgtctgagctgcgctgcaagcggttttacctttggtagctatgcaatgagctgggttcgtcaggcaccgggtaaaggtctggaatgggtgagcggtattagcggtagcggtggtagcacctattatgcagatagcgttaaaggtcgctttaccattagccgtgataatagcaaaaacaccctgtatctgcagatgaatagcctgcgtgcagaagataccgcagtttattattgtgcacgtgaaggtgaaaccagcttttatctggatgtttggggtcagggcaccctggttaccgttagcagc

SEQ ID NO: 474 - DNA, 333 bp, Homo Sapiens; identical to SEQ ID NO: 464

SEQ ID NO: 475 - DNA, 357 bp, Homo Sapiens
gaggtgcagctgttggagtctgggggaggcttggtacagcctggggggtccctgagactctcctgtgcagcctctggattcacctttggtgattatgctatgagctgggtccgccaggctccagggaaggggctggagtgggtctctggtattagtggtagtggtggtagcacatactacgcagactccgtgaagggccggttcaccatctccagagacaattccaagaacacgctgtatctgcaaatgaacagcctgagagccgaggacactgccgtgtattactgtgcgcgagaaggtgagacgagtttcggtctggacgtctggggccaaggtaccctggtcaccgtgactagt

SEQ ID NO: 476 - DNA, 333 bp, Homo Sapiens
cagtctgtgctgactcagccaccctcagcgtctgggacccccgggcagagggtcaccatctcttgttctggaagcagctccaacatcggaagtaattatgtatactggtatcagcagctcccaggaacggcccccaaactcctcatctatgacaataatgaccgaccctcaggggtccctgaccgattctctggctccaagtctggcacctcagcctccctggccatcagtgggctccggtccgaggatgaggctgattattactgtgtagcatgggatgacagcctgaatgggcattgggtgttcggcggaggaaccaagctgacggtccta

SEQ ID NO: 477 - DNA, 375 bp, Homo Sapiens
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcaggtattagtggtagtggtggtggcacatactacgcagactccgtgaagggccggttcaccatctccagagacaattccaagaacacgctgtatctgcaaatgaacagcctgagagccgaggacactgccgtgtattactgtgcgagagaaggtggcacatattactatggttcggggagttatgtacttgactactggggccagggtaccctggtcaccgtgactagt

SEQ ID NO: 478 - DNA, 330 bp, Homo Sapiens
cagtctgtgctgactcagccaccctcagcgtctgggacccccgggcagagggtcaccatctcttgttctggaagcagctccaacatcggaagtaattatgtatactggtatcagcagctcccaggaacggcccccaaactcctcatctataggaataatcaccggccctcaggggtccctgaccgattctctggctccaagtctggcacctcagcctccctggccatcagtgggctccggtccgaggatgaggctgattattactgtgcagcatgggatgacatcctgagtggttgggtgttcggcggaggaaccaagctgacggtccta

SEQ ID NO: 479 - DNA, 372 bp, Homo Sapiens
gaggtgcagctgttggagtctgggggaggcttggtacagcctgggggatccctgagactctcctgtgcagcctctggattcacctttagcagctatgccatgagctgggtccgccaggctccagggaaggggctggagtgggtctcaggtataagtggcagtggtggtagtacattctacgcagactccgtgaagggccggttcaccatctccagagacaattccgagaacacgctgtatctgcaaatgaacagcctgagagccgaggacactgccgtgtattactgtgcgagacagacgtcgagtggtctggcgggatactcttatggtttggacccctggggccagggtaccctggtcaccgtgactagt

SEQ ID NO: 480 - DNA, 333 bp, Homo Sapiens
cagtctgtgctgactcagccaccctcagcgtctgggacccccgggcagagggtcaccatctcctgcactgggagcagctccaacatcggggcaggttatgatgtacactggtatcagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcaggatgggatgacagcctgagtggttgggtgttcggcggaggaaccaagctgacggtccta

SEQ ID NO: 481 - DNA, 351 bp, Homo Sapiens
gaggtgcagctgttggagtctgggggaggcttggtacagcctggggggtccctgagactctcctgtgcagcctctggattcacctttagtgcctattggatggcctgggtccgccaggctccagggaaggggctggagtgggtctcatccattagtagtagtagtagttacatatactacgcagactcagtgaagggccgattcaccatctccagagacaattccaagaacacgctgtatctgcaaatgaacagcctgagagccgaggacactgccgtgtattactgtgcgagggtgacggagggtgggttagactactggggccaaggtaccctggtcaccgtgactagt

SEQ ID NO: 482 - DNA, 330 bp, Homo Sapiens
cagtctgtgctgactcagccaccctcagcgtctgggacccccgggcagagggtcaccatctcctgttctggaagcagctccaacatcggaaataatgctgtaaactggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgccagtcctatgacagcagcctgagtggttgggtgttcggcggaggaaccaagctgacggtccta

SEQ ID NO: 483 - DNA, 354 bp, Homo Sapiens
gaggtgcagctgttggagtctgggggaggcttggtacagcctggggggtccctgagactctcctgtgcagcctctggattcacctttagtagctatggcatgagctgggtccgccaggctccagggaaggggctggagtgggtctcaggtattagtggtagtggtggtagcacatactacgcagactccgtgaagggccggttcaccatctccagagacaattccaagaacacgctgtatctgcaaatgaacagcctgagagccgaggacactgccgtgtattactgtgcgagagacggtggctacgacgaactggactactggggccaaggtaccctggtcaccgtgactagt

SEQ ID NO: 484 - DNA, 333 bp, Homo Sapiens
cagtctgtgctgactcagccaccctcagcgtctgggacccccgggcagagggtcaccatctcctgcactgggagcagctccaacattggggcgggttatgttgtacattggtatcagcagctcccaggaacggcccccaaactcctcatctatgacaataataagcgaccctcaggggtccctgaccgattctctggctccaagtctggcacctcagcctccctggccatcagtgggctccggtccgaggatgaggctgattattactgtgcagcatgggatgacagcctgagtgcttgggtgttcggcggaggaaccaagctgacggtccta

SEQ ID NO: 485 - DNA, 351 bp, Homo Sapiens
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtagtagtagtggttacatatactacgcagactcactgaagggccgattcaccatctccagagacaattccaagaacacgctgtatctgcaaatgaacagcctgagagccgaggacactgccgtgtattactgtgccagagtgtggcggaatcaccttgactactggggccagggtaccctggtcaccgtgactagt

SEQ ID NO: 486 - DNA, 330 bp, Homo Sapiens
cagtctgtgctgactcagccaccctcagcgtctgggacccccgggcagagggtcaccatctcctgttctggaagcagctccgacatcggaagtaatactgtaaactggtatcagcagctcccaggaacggcccccaaactcctcatctatgacaataatcagcgaccctcaggggtccctgaccgattctctggctccaagtctggcacctcagcctccctggccatcagtgggctccggtccgaggatgaggctgattattactgccagtcctatgacagcagcctgagtggttgggtgttcggcggaggaaccaagctgacggtccta

SEQ ID NO: 487 - DNA, 351 bp, Homo Sapiens
gaggtgcagctgttggagtctgggggaggcttggtacagcctggggggtccctgagactctcctgtgcggcctctggattcaccttcagtaacgcctggatgagctgggtccgccaggctccagggaaggggctggagtgggtctctagtattcattggaatggcggttatataggttatgcagaatctgtgaagggccgattcaccatctccagagacaattccaagaacacgctgtatctgcaaatgaacagcctgagagccgaggacactgccgtgtattactgtgcgagagtaactctggacgcagttgactattggggccaaggtaccctggtcaccgtgactagt

SEQ ID NO: 488 - DNA, 330 bp, Homo Sapiens
cagtctgtgctgactcagccaccctcagcgtctgggacccccgggcagagggtcaccatctcctgttctggaagcagctccaacatcggaaataatgctgtaaactggtatcagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgccagtcatatgacaacagactgactgcttgggtgttcggcggaggaaccaagctgacggtccta

SEQ ID NO: 489 - protein, 225 aa, Homo Sapiens
EVQLLESGGGLVQPGGSLRLSCAASGFTFSSYWMSWVRQAPGKGLEWVSSISSSSSYIYYADSVKGRFTISRDNSKNTLYLQMNSLRAEDTAVYYCARVWRNYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 490 - protein, 216 aa, Homo Sapiens
QSVLTQPPSASGTPGQRVTISCSGSSSNIGSNTVNWYQQLPGTAPKLLIYSNNQRPSGVPDRFSGSKSGTSASLAITGLQAEDEADYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 491 - DNA, 678 bp, Homo Sapiens
gaagttcagctgctggaaagcggtggtggtctggttcagcctggtggtagcctgcgtctgagctgcgctgcaagcggttttacctttagcagctattggatgagctgggttcgtcaggcaccgggtaaaggtctggaatgggtgagcagcattagcagcagcagctcttatatttattatgccgatagcgtgaaaggtcgctttaccattagccgtgataatagcaaaaacaccctgtatctgcagatgaatagcctgcgtgcagaagataccgcagtttattattgtgcacgtgtgtggcgtaactatctggattattggggtcagggcaccctggttaccgttagcagcgcaagcaccaaaggtccgagcgtttttccgctggcaccgagcagcaaaagcaccagcggaggcaccgcagcactgggttgtctggttaaagattattttccggaaccggttaccgtgagctggaatagcggagcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgaaccgaaaagctgtgataaaacccatacctga

SEQ ID NO: 492 - DNA, 654 bp, Homo Sapiens
cagagcgttctgacccagcctccgagcgcaagcggtacaccgggtcagcgtgttaccattagctgtagcggtagcagcagcaatattggtagcaataccgttaattggtatcagcagctgcctggcaccgcaccgaaactgctgatttatagcaataatcagcgtccgagcggtgttccggatcgttttagcggtagcaaaagcggcaccagcgcaagcctggcaattaccggtctgcaggcagaagatgaagcagattattattgccagagctatgatagcagcctgagcggttgggtttttggtggtggcaccaaactgaccgttctgggtcagccgaaagcagcaccgagcgttaccctgtttccgcctagcagcgaagaactgcaggcaaataaagcaaccctggtttgtctgatcagcgatttttatccgggtgcagttaccgttgcatggaaagcagatagcagtccggttaaagccggtgttgaaaccaccaccccgagcaaacagagcaataataaatatgcagccagcagctatctgagcctgacaccggaacagtggaaaagccatcgtagctatagctgtcaggttacccatgaaggtagcaccgttgaaaaaaccgttgcaccgaccgaatgtagctgataa

SEQ ID NO: 493 - protein, 225 aa, Homo Sapiens
EVQLLESGGGLVQPGGSLRLSCAASGFTFSNAWMSWVRQAPGKGLEWVSSISSSSGYIYYADSLKGRFTISRDNSKNTLYLQMNSLRAEDTAVYYCARVWRNHLDYWGQGTLVTVTSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 494 - protein, 216 aa, Homo Sapiens
QSVLTQPPSASGTPGQRVTISCSGSSSDIGSNTVNWYQQLPGTAPKLLIYDNNQRPSGVPDRFSGSKSGTSASLAISGLRSEDEADYYCQSYDSSLSGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 495 - DNA, 678 bp, Homo Sapiens
gaggtgcagctgttggagtctgggggaggcttggtacagcctggggggtccctgagactctcctgtgcagcctctggattcaccttcagtaacgcctggatgagctgggtccgccaggctccagggaaggggctggagtgggtctcatccattagtagtagtagtggttacatatactacgcagactcactgaagggccgattcaccatctccagagacaattccaagaacacgctgtatctgcaaatgaacagcctgagagccgaggacactgccgtgtattactgtgccagagtgtggcggaatcaccttgactactggggccagggtaccctggtcaccgtgactagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgataaaacccatacctga

SEQ ID NO: 496 - DNA, 654 bp, Homo Sapiens
cagtctgtgctgactcagccaccctcagcgtctgggacccccgggcagagggtcaccatctcctgttctggaagcagctccgacatcggaagtaatactgtaaactggtatcagcagctcccaggaacggcccccaaactcctcatctatgacaataatcagcgaccctcaggggtccctgaccgattctctggctccaagtctggcacctcagcctccctggccatcagtgggctccggtccgaggatgaggctgattattactgccagtcctatgacagcagcctgagtggttgggtgttcggcggaggaaccaagctgacggtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatgatga

SEQ ID NO: 497 - protein, 5 aa, Homo sapiens
  VARIANT (1)..(1): X = D or S
XYAMS

SEQ ID NO: 498 - protein, 10 aa, Homo sapiens
  VARIANT (1)..(1): X = Q or E
XGXTSFXLDV

SEQ ID NO: 499 - protein, 13 aa, Homo sapiens
  VARIANT (1)..(1): X = C or S
XGSXSNIGXXYVY

SEQ ID NO: 500 - protein, 7 aa, Homo sapiens
  VARIANT (3)..(3): X = N or R
DNXXRPS

SEQ ID NO: 501 - protein, 12 aa, Homo sapiens
  VARIANT (5)..(5): X = D, V or W
VAWDXSXXGXWV

SEQ ID NO: 502 - protein, 5 aa, Homo sapiens
  VARIANT (1)..(1): X = D, N or S
XXWMS

SEQ ID NO: 503 - protein, 17 aa, Homo sapiens
  VARIANT (4)..(4): X = S or T
SISXSSXYIYYADXXKG

SEQ ID NO: 504 - protein, 8 aa, Homo sapiens
  VARIANT (5)..(5): X = Y, S, A or H
VWRNXLXY

SEQ ID NO: 505 - protein, 13 aa, Homo sapiens
  VARIANT (6)..(6): X = N or D
SGSSSXIGSNXVN

SEQ ID NO: 506 - protein, 7 aa, Homo sapiens
  VARIANT (1)..(1): X = D or S
XNNQXPX

SEQ ID NO: 507 - protein, 11 aa, Homo sapiens
  VARIANT (4)..(4): X = D or S
QSYXSSLXGWV

SEQ ID NO: 508 - protein, 446 aa, Homo sapiens
EVQLLESGGGLVQPGGSLRLSCAASGFTFSSYWMSWVRQAPGKGLEWVSSISSSSSYIYYADSVKGRFTISRDNSKNTLYLQMNSLRAEDTAVYYCARVWRNYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 509 - protein, 216 aa, Homo sapiens; identical to SEQ ID NO: 490

(19) United States
(12) Patent Application Publication
(10) Pub. No.: US 2014/0050743 A1
(43) Pub. Date: Feb. 20, 2014

(54) BINDING PROTEINS TO INHIBITORS OF COAGULATION FACTORS

(71) Applicants: Frank Dittmer, Dusseldorf (DE); Anja Buchmüller, Essen (DE); Christoph Gerdes, Koln (DE); Adrian Tersteegen, Wuppertal (DE); Mark Jean Gnoth, Mettmann (DE); Lars Linden, Dusseldorf (DE); Axel Harrenga, Wuppertal (DE); Joanna Grudzinska-Goebel, Berlin (DE); Mario Jeske, Solingen (DE); Martina Schäfer, Berlin (DE); Jörg Birkenfeld, Frankfurt am Main (DE); Holger Paulsen, Hilden (DE); Ricarda Finnern, Aachen (DE); Anke Mayer-Bartschmid, Wulfrath (DE); Andrea Eicker, Monchengladbach (DE); Simone Greven, Dormagen (DE); Susanne Steinig, Koln (DE)

(72) Inventors: Frank Dittmer, Dusseldorf (DE); Anja Buchmüller, Essen (DE); Christoph Gerdes, Koln (DE); Adrian Tersteegen, Wuppertal (DE); Mark Jean Gnoth, Mettmann (DE); Lars Linden, Dusseldorf (DE); Axel Harrenga, Wuppertal (DE); Joanna Grudzinska-Goebel, Berlin (DE); Mario Jeske, Solingen (DE); Martina Schäfer, Berlin (DE); Jörg Birkenfeld, Frankfurt am Main (DE); Holger Paulsen, Hilden (DE); Ricarda Finnern, Aachen (DE); Anke Mayer-Bartschmid, Wulfrath (DE); Andrea Eicker, Monchengladbach (DE); Simone Greven, Dormagen (DE); Susanne Steinig, Koln (DE)

(73) Assignee: BAYER INTELLECTUAL PROPERTY GMBH, Monheim (DE)

(21) Appl. No.: 13/980,431

(22) PCT Filed: Jan. 17, 2012
(86) PCT No.: PCT/EP2012/050595

§ 371 Date: Nov. 5, 2013

(30) Foreign Application Priority Data
Jan. 19, 2011 (EP) 11151410.5

(51) Int. Cl. C07K 16/44

(52) U.S. Cl. CPC C07K 16/44
USPC 424/175.1; 530/389.8; 530/388.9; 530/387.3; 536/23.53; 435/320.1; 435/252.3; 435/254.2; 435/345; 544/137

(57) ABSTRACT

The present invention relates to the identification and use of antigen-binding regions, antibodies, antigen-binding antibody fragments and antibody mimetics, neutralizing the anti-coagulant effect of an anticoagulant in vitro and/or in vivo. Antibodies and functional fragments of the invention and antibody mimetics can be used to specifically reverse the pharmacological effect of an anticoagulant e.g. a FXa inhibitor for therapeutic (antidote) and/or diagnostic purposes. The invention also provides nucleic acid sequences encoding foregoing molecules, vectors containing the same, pharmaceutical compositions and kits with instructions for use.

The present invention relates to the identification and use of antigen-binding regions, antibodies, antigen-binding antibody fragments and antibody mimetics interacting with and neutralizing therapeutic inhibitors of coagulation factors.

Antibody mimetics, antibodies and functional fragments of the invention can be used to specifically reverse the pharmacological effect of e.g. the FXa inhibitor for therapeutic (antidote) and/or diagnostic purposes. The invention also provides nucleic acid sequences encoding foregoing molecules, vectors containing the same, pharmaceutical compositions and kits with instructions for use.

### BACKGROUND OF THE INVENTION

A general limitation of anticoagulant drugs is the bleeding risk associated with the treatment and the limited ability to rapidly reverse the activity in case of an emergency situation. Although the emerging anticoagulant rivaroxaban is a novel drug with proven tolerability and safety, the availability of a specific agent allowing rapid neutralization of its effect (antidote), would be medically advantageous. Here, we describe novel specific antibodies, antigen-binding antibody fragments and antibody mimetics, which allow the rapid reversal of anticoagulation induced by FXa inhibitors, e.g. rivaroxaban, thereby acting as a selective antidote.

Thromboembolic disorders such as deep vein thrombosis (DVT), pulmonary embolism (PE), stroke and myocardial infarction are leading causes of cardiovascular-associated morbidity and death. Since many years, treatment and prevention of thromboembolism in high risk patients by anticoagulant drugs like vitamin K antagonists (VKA, e.g. warfarin), unfractionated heparin (UFH) and low molecular weight heparin (LMWH) are widely established medical interventions. However, a general limitation of anticoagulant drugs is the bleeding risk associated with the treatment and the limited ability to rapidly reverse the activity in case of an emergency situation. For the classic anticoagulant agents certain antidotes are available, like protamine sulfate (for UFH and LMWH) and vitamin K (for VKA). In addition recombinant Factor VIIa or blood products can be taken into consideration as unspecific reversal agents. However, there are no specific antidotes available or in cinical development for emerging oral anticoagulants (e.g. rivaroxaban). These new anticoagulants will become of increasing importance in the upcoming years and the availability of a specific antidote for one of the new anticoagulants would provide a medical advantage in emergency situations.

Despite the availability of conventional anticoagulant drugs like UFH, LMWH and VKA, there exists a high medical need for improved therapeutics with predictable pharmacokinetics, better therapeutic window and convenient application. Rivaroxaban is an emerging orally available anticoagulant agent, directly inhibiting the blood coagulation factor Xa (FXa) (Perzborn E. et al., Nat. Rev. Drug Discov. 2011, 10(1):61-7). FXa represents a key enzyme of the coagulation cascade, catalyzing the clot formation by the generation of thrombin from prothrombin. Rivaroxaban (chemical name: 5-Chloro-N-[[(5S)-2-oxo-3[4-(3-oxomorpholin-4-yl)-phenyl]-1,3-oxazolidin-5-yl]methyl]thiophene-2-carboxamide) has a molecular weight of 436 g/mol and inhibits FXa dose-dependently (Kᵢ of 0.4 nM) with a >10,000 higher selectivity than for other biologically relevant serine proteases. It has a rapid onset of action (kₒₙ of 1.7×10/M−1 s−1) and binds reversible (k_{off} of 5×10−3 s−1). Rivaroxaban inhibits also prothrombinase-bound (IC₅₀ of 2.1 nM) and clot associated FXa (IC₅₀ of 75 nM).and shows dose-dependently antithrombotic activity in a variety of animal models on venous and arterial thrombosis. In clinical studies, rivaroxaban showed a favorable safety and tolerability profile and was effective in preventing VTE in adult patients following elective hip or knee replacement surgery. Rivaroxaban is marketed under the brand name Xarelto® for VTE prevention in adult patients following elective hip or knee replacement surgery, and it is so far the only new oral anticoagulant that has consistently demonstrated superior efficacy over enoxaparin for this indication. The compound is also being developed for chronic indications like for the prevention of stroke in high risk atrial fibrillation patients.

Besides rivaroxaban, there are a number of other orally available direct FXa inhibitors under various stages of clinical development, including apixaban, edoxaban, betrixaban, darexaban, and TAK-442 (Garcia, D. et al., Blood 2010; 115(1):15-20). In addition, the pentasaccharide Fondaparinux, an indirect (antithrombin-dependent) parental FXa inhibitor, is approved for the prevention and treatment of VTE. Another new class of anticoagulants are direct thrombin inhibitors (DTI) binding to the active site of thrombin thereby blocking its fibrin interaction. However, due to the absence of specific antidotes for all these drugs, bleeding risks and the inability to rapidly reverse anticoagulation prior to urgent surgery or vascular intervention remain important concerns when administering any anticoagulant. Consequently, offering a specific pair of anticoagulant and antidote would address an important unmet medical need.

Rivaroxaban is a drug with proven tolerability and safety as well as a compound with relatively short half-life. However, dependent on the severity of a putative clinical bleeding situation the mere cessation of medication may be not sufficient to reverse its anticoagulant effect. The availability of a specific antidote would be advantageous in rare emergency situations, where the rapid neutralization of the anticoagulant effect is required either as a result of a severe bleeding event (e.g. caused by trauma) or due to a need for an urgent invasive procedure (e.g. an emergency surgery). Currently, in case of life-threatening bleeding, administration of recombinant factor VII may be considered, however there is only limited non-clinical data and clinical data available (Levi, M. et al., N Engl J. Med. 2010; 363(19):1791-800). Non-specific antidotes which might be taken into consideration are blood-derived (activated) prothrombin complex concentrate (aPCC, PCC) or fresh frozen plasma. However, it is important to note that there is no clinical experience with any of these reversal strategies and these interventions inherit medical issues like a prothrombotic risk, a risk of infections or a slow onset of action (Romualdi et al., Curr. Pharm. Des. 2010; 16(31):3478-82).

Recently, pre-clinical data for PRT064445, a non-selective antidote of FXa inhibitors, has been reported. This molecule is based on a mutated recombinant version of the FXa protein, lacking its intrinsic procoagulant activity but still being able to bind to different types of FXa active site inhibitors, thereby neutralizing their anticoagulant effect (WO 2009/042962 A2). The compound has been reported in the preclinical phase for reversal of anticoagulation of all current FXa inhibitors, both small molecule and anti-thrombin dependent. However, a disadvantage of such a non-selective antidote is that its use would lead to the lack of effectivity of all FXa inhibitors, which could be problematic in case a prompt anticoagulation of the treated patient would be necessary. Moreover, the development of anti-drug antibodies cross-specific to the endogenous FXa-protein can not be excluded.

Thus to overcome the aforementioned problems an ideal antidote to coagulation inhibitors e.g. FXa inhibitors containing the structural element of formula 1 (e.g. rivaroxaban) would be highly specific allowing further subsequent treatment with a different inhibitor or with an other inhibitor of a different compound class, if necessary. Its affinity to the drug should be below μM range in order to allow for an efficient and sustained reduction of unbound inhibitor. Moreover, it should have a rapid onset of action and should be devoid of any intrinsic influence on the coagulation cascade. In addition, a short half life would be of advantage to allow a fast re-uptake of medicamentation. Furthermore, the antidote should be devoid of the other described inherit medical issues like a prothrombotic risk or a risk of infections.

The solution is the provision of an antibody or antigen-binding fragment thereof or an antibody mimetic neutralizing the anti-coagulant activity of an anticoagulant.

It has been described that antibodies and antibody-mimetics are able to specifically bind to small molecules with a molecular weight below 1000 Da, so called haptens. Binding and neutralization of small molecular compounds by intravenously administered antibody fragments (Fab) derived from sheep polyclonal sera has been established e.g. for the treatment of digoxin intoxication (DigiFab, Digoxin immune Fab (ovine)) or for the use as an antivenom (CroFab, Crotalidae polyvalent immune Fab (ovine)).

Recently, generation of hapten-specific antibodies has also been reported using recombinant antibody technologies (reviewed in: Sheedy, C. et al., Biotech Adv 2007; 25:333-52). Based on highly diverse phage-display libraries comprising more than ˜10¹⁰ different antibody molecules, hapten-specific binder with up to sub-nanomolar affinities could be isolated for various classes of small molecules (Vaughan et al, Nat. Biotech. 1996; 14 (3):309-314). Nevertheless, haptens remain challenging targets and anti-hapten antibodies are often of lower affinity than those of high molecular weight antigens like proteins. This is due to their small and hydrophobic nature, providing only few functional groups which can interact with the antibody-binding site (paratope). Furthermore, the isolation of hapten-specific antibodies from display-libraries is hampered by the need of chemical modification of the molecule in order to immobilize the target during the “biopanning” step.

It should be mentioned that the concept of hapten-specific binding proteins recently has been extended to engineered ligand binding proteins (so called “antibody mimetics”). In this regard, a digoxigenin-binding engineered lipocalin (anticalin) was described, suitable as a digoxin antidote during digitalis intoxication (Schlehuber S, and Skerra A., Drug Discov. Today 2005; 10 (1):23-33).

Provided herein are antibodies, antigen-binding antibody fragments thereof, or variants thereof, or antibody mimetics that bind with high affinity to FXa inhibitors comprising structure formula 1. Also provided are therapies based on antibody, antigen binding antibody-fragment and antibody mimetics aiming at the reversal of the pharmacological effect of these compounds. Also provided are methods based on antibody, antigen binding antibody-fragment and antibody mimetics aiming at the functional neutralization of these FXa inhibitors in blood samples for diagnostic purposes.

### SUMMARY

It is an object of the present invention to provide antibodies, or antigen-binding antibody fragments thereof, or antibody mimetics which neutralize therapeutic inhibitors of a coagulation factor and thus are useful for the reversal of their anticoagulant activity for therapeutic and/or diagnostic purposes.

It is a further object of the present invention to provide human antibodies, or antigen-binding antibody fragments thereof, or antibody mimetics which bind therapeutic inhibitors of the coagulation factor Xa (FXa) containing the structural element given in formula 1 (e.g. rivaroxaban) and thus are useful for the reversal of their anticoagulant activity for therapeutic and/or diagnostic purposes.

The invention is based on the surprising discovery that by methods of antibody phage display, antibodies or fragments thereof specific to compounds comprising a group of formula 1 could be identified that do not bind to other FXa-inhibitors. Thus, the antibodies useful as specific antidotes will allow a restart of anticoagulation of the treated subjects with these other FXa inhibitors if needed.

According to a first aspect of the present invention it was possible to synthesize derivatives of rivaroxaban allowing their immobilization on surfaces based on the biotin-streptavidin interaction. Immobilization of rivaroxaban and its derivatives is a prerequisite for the selection of antibodies from phage libraries (phage panning) and for screening and analyses of specific antibodies in the ELISA-format.

According to a second aspect of the invention it was possible to identify antibodies and antibody fragments with affinities of K_{D} <500 nM and with half-maximal effective concentrations (EC50) in a biochemical FXa-assay inhibited with rivaroxaban of EC₅₀ <2 μM.

According to a third aspect of the present invention, it was possible to identify antibodies and antibody fragments thereof specific for compounds containing the structural element given in formula 1, which do not crossreact with other inhibitors of FXa like apixaban, edoxaban or razaxaban.

The present invention relates to a therapeutic method of selectively neutralizing the effect of a coagulation inhibitor in a subject undergoing anticoagulant therapy by administering to the subject an effective amount of antibody or antigen-binding fragment thereof or antibody mimetic.

One embodiment of the invention is directed to an isolated antibody or antigen-binding fragment thereof as depicted in table 1

In another aspect, the antibodies, or antigen-binding antibody fragments thereof, or antibody mimetics are co-administered with an agent capable of extending the plasma half-life (or circulating half-life). In yet another aspect, the antibody, or antigen-binding antibody fragment thereof, or antibody mimetic is conjugated to itself or to other moieties to extend its plasma half-life.

Also provided are pharmaceutical compositions which contain the antibody, antigen-binding fragment thereof, or antibody mimetic.

In another aspect, this invention provides a kit comprising rivaroxaban and an antibody or antigen-binding fragment thereof depicted in table 1 for use when substantial neutralization of the FXa inhibitor's anticoagulant activity is needed.

In yet another embodiment an isolated prokaryotic or eukaryotic host cell comprising a polynucleotide encoding a polypeptide of the invention is provided.

These and other features, aspects, and advantages of the present invention will become better understood with reference to the following description and claims.

An antibody of the invention may be an IgG (e.g., IgG₁ IgG2, IgG3, IgG4), while an antigen binding antibody fragment may be a Fab, Fab′, F(ab′)₂ or scFv, for example. An inventive antigen binding antibody fragment, accordingly, may be, or may contain, an antigen-binding region that behaves in one or more ways as described herein.

The invention also is related to isolated nucleic acid sequences, each of which can encode an aforementioned antibody or antigen-binding fragment thereof that is specific for a compound comprising a group of the formula 1. Nucleic acids of the invention are suitable for recombinant production of antibodies or antigen-binding antibody fragments. Thus, the invention also relates to vectors and host cells containing a nucleic acid sequence of the invention.

Compositions of the invention may be used for therapeutic, prophylactic or diagnostic applications. The invention, therefore, includes a pharmaceutical composition comprising an inventive antibody or antigen-binding fragment thereof and a pharmaceutically acceptable carrier or excipient therefore. In a related aspect, the invention provides a method for the neutralization of rivaroxaban in conditions associated with the undesired presence of rivaroxaban. In a preferred embodiment the aforementioned condition is a situation, where the rapid rerversal of the anticoagulant effect in patients is required (e.g. due to a need for an urgent invasive procedure). Such method contains the steps of administering to a subject in need thereof an effective amount of the pharmaceutical composition as described or contemplated herein.

An antibody, antigen-binding fragment thereof or antibody mimetic of the invention can be used in diagnostic methods to determine the presence and/or quantity of a FXa inhibitor.

The invention also provides instructions for using an antibody library to isolate one or more members of such library that binds specifically to compounds containing the structural component described by formula 1.

### DESCRIPTION OF THE FIGURES

FIG. 1 shows the results of the functional neutralization of rivaroxaban by the Fab M18-G08-G-DKTHT in a biochemical FXa-assay (described in Example 4). To assess the functional potency of the Fab with regard to neutralization of rivaroxaban a biochemical FXa-assay was performed. Increasing concentrations of Fab were premixed with a fluorogenic FXa substrate and were added to a premixed solution of FXa (0.05 nM) with rivaroxaban (0.6 nM, IC₇₅). Analysis of the reaction progress curves, recorded for 50 min, resulted in an EC50 of 6 nM for the reversal of rivaroxaban induced FXa inhibition by the Fab M18-G08-G-DKTHT. x-axis: t (min), y-axis: relative fluorescence units; dotted line: buffer; filled triangles: factor Xa control; filled squares: factor Xa+0.6 nM rivaroxaban;

Diamonds: factor Xa+0.6 nM rivaroxaban+0.46 nM Fab
Bars: factor Xa+0.6 nM rivaroxaban+1.4 nM Fab
Crosses: factor Xa+0.6 nM rivaroxaban+4.1 nM Fab
Open squares: factor Xa+0.6 nM rivaroxaban+12 nM Fab
Circles: factor Xa+0.6 nM rivaroxaban+37 nM Fab
Stars: factor Xa+0.6 nM rivaroxaban+110 nM Fab
Open triangles: factor Xa+0.6 nM rivaroxaban+330 nM Fab

FIG. 2 shows the Rosenthal-Scatchard plot describing the binding of various concentrations of rivaroxaban to 0.5 μM Fab M18-G08-G-DKTHT (described in Example 7). The determination of the unbound concentration of rivaroxaban (fraction unbound=fu) in the presence of M18-G08-G-DKTHT after ultrafiltration allows the determination of the K_{D} value of M18-G08-G-DKTHT towards rivaroxaban. The K_{D} value of about 0.48 nM was calculated from the slope of the Rosenthal-Scatchard plot. Y axis: (fraction bound)/(fraction unbound); X axis: (fraction bound)*(concentration of rivaroxaban [μM]).

FIG. 3 shows results from a thrombin generation assay in human platelet poor plasma (described in Example 8) in the absence (FIG. 3a) or presence of 0.1 μM rivaroxaban (FIG. 3b-d) with or without Fab M18-G08-G-DKTHT (0 μM (FIG. 3b), 0.09 μM (FIG. 3c) and 0.72 μM (FIG. 3d)). It can be concluded that M18-G08-G-DKTHT neutralizes concentration-dependently the effect of rivaroxaban on thrombin generation in human plasma.

FIG. 4 shows results from a thrombin generation assay in human platelet poor plasma (described in Example 8) in the absence (FIG. 4a) or presence of 0.1 μM SATI (FIG. 4b-d) with or without Fab M18-G08-G-DKTHT (0 μM (FIG. 4b), 0.09μM (FIG. 4c) and 0.72μM (FIG. 4d)). It can be concluded that M18-G08-G-DKTHT neutralizes concentration-dependently the effect of SATI on thrombin generation in human plasma (X axis: time [min]; Y axis: thrombin [nM]).

FIG. 5 shows results from a thrombin generation assay in human platelet poor plasma (described in Example 8) in the absence of any FXa inhibitor with or without Fab M18-G08-G-DKTHT (0 μM (FIG. 5a), 0.09μM (FIG. 5b) and 0.72μM (FIG. 5c)). It can be concluded that M18-G08-G-DKTHT itself has no effect on thrombin generation in human plasma (X axis: time [min]; Y axis: thrombin [nM]).

FIG. 6 shows results from a plasma-based FXa assay (described in Example 9) in the presence of 0.05 μM rivaroxaban without or with increasing concentrations of Fab M18-G08-G-DKTHT (0-1000 nM). It could be demonstrated that increasing concentrations of M18-G08-G-DKTHT potently reverse the inhibitory effect of rivaroxaban on FXa in human plasma. X axis: M18-G08-G-DKTHT [nM]; Y axis: FXa activity [%]; black bar: Control (no rivaroxaban, no M18-G08-G-DKTHT); grey bar: no M18-G08-G-DKTHT; chequered bars: increasing concentrations [nM] M18-G08-G-DKTHT from left to right: 0.01-0.1-1-10-100-1000.

FIG. 7 shows results from a prothrombin (PT) assay in human plasma (described in Example 10) in the presence of 0.17 (open symbols) and 0.33 μM (filled symbols) rivaroxaban, respectively. It could be demonstrated that increasing concentrations of M18-G08-G-DKTHT potently reverse the inhibitory effect of rivaroxaban on PT in human plasma. (X axis: concentration of M18-G08-G-DKTHT [log M]; Y axis: prothrombin time [sec]; data represent final assay concentrations with means±sem of 5 experiments).

FIG. 8 shows results from a prothrombin (PT) assay in rat plasma (described in Example 10) in the presence of 0.4 (open symbols) and 0.8 μM (filled symbols) rivaroxaban, respectively. It could be demonstrated that increasing concentrations of M18-G08-G-DKTHT potently reverse the inhibitory effect of rivaroxaban on PT in human plasma. (X axis: concentration of M18-G08-G-DKTHT [log M]; Y axis: prothrombin time [sec]; data represent final assay concentrations with means±sem of 5 experiments).

FIG. 9 depicts an SDS-PAGE of purified non reduced (−) and reduced (+) Fab fragment M18-G08-G-DKTHT. Purification is described in Example 16. LC=light chain, HC=heavy chain, Fab=intact Fab fragment, the very right lane contains Precision A11 Blue molecular weight marker (BioRad).

FIG. 10 shows results of a rat PK/PD study (described in Example 17) in which PT in rat plasma was assayed ex vivo after oral dosing of rivaroxaban (at time point 0) and infusion of M18-G08-G-DKTHT for 1 hour from 1.5 to 2.5 h (chequered box). A rapid and sustained normalization of PT values could be demonstrated (X axis: time after oral dosing of rivaroxaban in h; Y axis: prothrombin time in sec; data represent means±sem of 5 animals). Filled squares: vehicle control; open squares: rivaroxaban (1.5 mg/kg); filled triangles: rivaroxaban (1.5 mg/kg) plus M18-G08-G-DKTHT (85 mg/kg).

FIG. 11 shows a concentration/time profile of unbound rivaroxaban in rat plasma following oral administration of 1.5 mg/kg rivaroxaban and infusion of 85 mg/kg Fab M18-G08-G-DKTHT over 1 h starting 1.5 h after administration of rivaroxaban (described in Example 18). The study was performed in both, conscious (dashed line) and anesthetized rats (dotted line). In control rats (anasthetized) only rivaroxaban was administered (solid line). A rapid reduction of the plasma concentration of unbound rivaroxaban following infusion of M18-G08-G-DKTHT is demonstrated. For some samples the concentration of unbound rivaroxaban could not be determined because their values were below the lower limit of quantification (LLOQ; grey horizontal line). X axis: time in h; Y axis: concentration of unbound rivaroxaban in μg/L.

FIG. 12 shows the effect of M18-G08-G-DKTHT on cumulative tail bleeding time prolonged by rivaroxaban (1 mg/kg i.v.) in anesthetized rats (described in Example 19). It could be demonstrated that M18-G08-G-DKTHT at an equimolar dose of 107.5 mg/kg significantly shortens the bleeding time prolonged by rivaroxaban to almost normal values. Horizontal bars indicate group medians. P-values are from Kruskal-Wallis test followed by Dunn's multiple comparison. Filled squares: untreated; filled circles: rivaroxaban (1 mg/kg); open circles: rivaroxaban (1 mg/kg) plus M18-G08-G-DKTHT (107.5 mg/kg); Y axis: cumulative bleeding time in sec.

FIG. 13 depicts a cartoon representation of the Fab M18-G08-G-DKTHT in complex with rivaroxaban shown in sticks (described in Example 21).

FIG. 14 depicts binding and interaction of Fab M18-G08-G-DKTHT with rivaroxaban (described in Example 21).

FIG. 15 shows the results of a competition ELISA (described in Example 22). A fixed amount of Fab M18-G08-G-DKTHT was preincubated with various concentrations of rivaroxaban and residual binding of the Fab to coated compound from Example 1K was determined X axis: concentration of rivaroxaban in μM; Y axis: OD₄₀₅ signal.

FIG. 16 shows results from a thrombin generation assay in human platelet poor plasma (described in Example 23) in the absence (FIG. 16a) or presence of 3 μM apixaban (FIG. 16b-d) with or without Fab M18-G08-G-DKTHT (0 μM (FIG. 16b), 1.43 μM (FIG. 16c-d) and 0.1 μM rivaroxaban (FIG. 16d)). It can be seen that M18-G08-G-DKTHT does not influence the anticoagulative effect of apixaban (X axis: time [min]; Y axis: thrombin [nM]).

FIG. 17 shows results from a thrombin generation assay in human platelet poor plasma (described in Example 23) in the absence (FIG. 17a) or presence of 0.75 μM dabigatran (FIG. 17b-d) with or without Fab M18-G08-G-DKTHT (0 μM (FIG. 17b), 0.72 μM (FIG. 17c-d) and 0.1 μM rivaroxaban (FIG. 17d)). It can be observed that M18-G08-G-DKTHT does not influence the anticoagulative effect of dabigatran (X axis: time [min]; Y axis: thrombin [nM]).

### DETAILED DESCRIPTION OF THE INVENTION

The present invention is based on the discovery of antibodies and antibody fragments that are specific to or have a high affinity for FXa inhibitors including compounds comprising a group of the formula 1 and can deliver a therapeutic benefit to a subject. The antibodies of the invention may be human, humanized or chimeric. The present invention is further illustrated in the following examples which are not intended to be in any way limiting to the scope of the invention as claimed.

### DEFINITIONS

A “human” antibody or antigen-binding fragment thereof is hereby defined as one that is not chimeric (e.g., not “humanized”) and not from (either in whole or in part) a non-human species. A human antibody or antigen-binding fragment thereof can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained there from. Another example of a human antibody or antigen-binding fragment thereof is one that is encoded by a nucleic acid isolated from a library of antibody sequences of human origin (e.g., such library being based on antibodies taken from a human natural source). Examples of human antibodies include antibodies as described in Söderlind et al., Nat. Biotechnol. 2000, 18(8):853-856.

A “humanized antibody” or humanized antigen-binding fragment thereof is defined herein as one that is (i) derived from a non-human source (e.g., a transgenic mouse which bears a heterologous immune system), which antibody is based on a human germline sequence; (ii) where amino acids of the framework regions of a non human antibody are partially exchanged to human amino acid sequences by genetic engineering or (iii) CDR-grafted, wherein the CDRs of the variable domain are from a non-human origin, while one or more frameworks of the variable domain are of human origin and the constant domain (if any) is of human origin.

A “chimeric antibody” or antigen-binding fragment thereof is defined herein as one, wherein the variable domains are derived from a non-human origin and some or all constant domains are derived from a human origin.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. The term monoclonal antibody specifically includes chimeric, humanized and human antibodies.

As used herein, an antibody “binds specifically to”, is “specific to/for” or “specifically recognizes” an antigen of interest, e.g. a small molecule hapten (here, FXa inhibitors comprising structure formula 1, e.g. rivaroxaban), is one that binds the antigen with sufficient affinity such that the antibody is useful as a therapeutic agent in neutralizing its target in plasma samples, and does not significantly crossreact with other FXa inhibitors than those containing the structural component described in formula 1. The term “specifically recognizes” or “binds specifically to” or is “specific to/for” a particular target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10′ M, alternatively less than about 10⁻⁸ M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”. “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxidase and tetramethyl benzidine with hydrogen peroxide). The reaction in certain wells is scored by the optical density, for example, at 450 nm. Typical background (=negative reaction) may be 0.1 OD; typical positive reaction may be 1 OD. This means the difference positive/negative is more than 5-fold, 10-fold, 50-fold, and preferably more than 100-fold. Typically, determination of binding specificity is performed by using not a single reference antigen, but a set of about three to five unrelated antigens, such as milk powder, BSA, transferrin or the like.

“Binding affinity” refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules Affinity can be measured by common methods known in the art, including those described herein. In one embodiment, the “K_{D}” or “K_{D} value” according to this invention is measured by using surface plasmon resonance assays using a Biacore T100 instrument (GE Healthcare Biacore, Inc.) according to Example 5. The dissociation equilibrium constant (K_{D}) was calculated based on the ratio of association (kₒₙ) and dissociation rated (k_{off}) constants, obtained by fitting sensograms with a first order 1:1 binding model using Biacore Evaluation Software. Other suitable devices are BIACORE(R)-2000, a BIACORE (R)-3000 (BIAcore, Inc., Piscataway, N.J.), or ProteOn XPR36 instrument (Bio-Rad Laboratories, Inc.).

In another embodiment, the “K_{D}” or “K_{D} value” according to this invention is measured by using Isothermal Titration calorimetry (ITC) with control and analysis software (Microcal/GE Healthcare, Freiburg, Germany) according to Example 6. Heat released during the binding reaction in solution is monitored over time and thermodynamic data is analyzed using the analysis software to estimate the K_{D}-value. Isothermal Titration calorimetry with control and analysis software (Microcal/GE Healthcare, Freiburg, Germany) according to Example 6.

In yet another embodiment, the “K_{D}” or “K_{D} value” according to this invention is determined by measuring the unbound concentration of antigen in the presence of a fixed amount of antibody or antibody fragment in solution. The K_{D} value is calculated using the Rosenthal-Scatchard plot according to Example 7. In this method, the X-axis is the concentration of bound ligand and the Y-axis is the concentration of bound ligand divided by the concentration of unbound ligand. It is possible to estimate the K_{D} from a Rosenthal-Scatchard plot, as the K_{D} is equal to the negative reciprocal of the slope.

The term “antibody”, as used herein, is intended to refer to immunglobulin molecules, preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs and up to four FRs. arranged from amino terminus to carboxy-terminus e.g. in the following order: FR1, CDR1, FR2, CDR2, FR3, CDR3, FR4.

As used herein, the term “Complementarity Determining Regions (CDRs; e.g., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (e.g. about residues 24-34 (L1), 50-56 (L2) and 89-97 (L3) in the light chain variable domain and 31-35 (H1), 50-65 (H2) and 95-102 (H3) in the heavy chain variable domain; (Kabat et al., Sequences of Proteins of Immulological Interest, 5th Ed. Public Health Service, National Institutes of Health, Bethesda, Md. (1991)) and/or those residues from a “hypervariable loop” (e.g. about residues 26-32 (L1), 50-52 (L2) and 91-96 (L3) in the light chain variable domain and 26-32 (H1), 53-55 (H2) and 96-101 (H3) in the heavy chain variable domain (Chothia and Lesk; J Mol Biol 196: 901-917 (1987)). In some instances, a complementarity determining region can include amino acids from both a CDR region defined according to Kabat and a hypervariable loop.

Depending on the amino acid sequence of the constant domain of their heavy chains, intact antibodies can be assigned to different “classes”. There are five major classes of intact antibodies: IgA, IgD, IgE, IgG, and IgM, and several of these maybe further divided into “subclasses” (isotypes), e.g., IgG1, IgG2, IgG3, IgG4, IgA, and IgA2. The heavy-chain constant domains that correspond to the different classes of antibodies are called [alpha], [delta], [epsilon], [gamma], and [mu], respectively. The subunit structures and three-dimensional configurations of different classes of immunglobulins are well known. As used herein antibodies are conventionally known antibodies and functional fragments thereof.
A “functional fragment” or “antigen-binding antibody fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hyper variable region(s) of an antibody, e.g., the CDR1, -2, and/or −3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320).
“Functional fragments” or “antigen-binding antibody fragments” of the invention include Fab, Fab′, F(ab′)₂, and Fv fragments; diabodies; single domain antibodies (DAbs), linear antibodies; single-chain antibody molecules (scFv); and multispecific, such as bi- and tri-specific, antibodies formed from antibody fragments (C. A. K Borrebaeck, editor (1995) Antibody Engineering (Breakthroughs in Molecular Biology), Oxford University Press; R. Kontermann & S. Duebel, editors (2001) Antibody Engineering (Springer Laboratory Manual), Springer Verlag). An antibody other than a “multi-specific” or “multi-functional” antibody is understood to have each of its binding sites identical. The F(ab′)2 or Fab may be engineered to minimize or completely remove the intermolecular disulphide interactions that occur between the CH1 and CL domains. A preferred class of antigen-binding fragments for use in the present invention is a Fab fragment.

An antibody and antigen-binding fragment thereof of the invention may be derived from a recombinant antibody library that is based on amino acid sequences that have been isolated from the antibodies of a large number of healthy volunteers. Using the n-CoDeR® technology the fully human CDRs are recombined into new antibody molecules (Soderling et al., Nat. Biotech. 2000, 18:853-856). The unique recombination process allows the library to contain a wider variety of antibodies than could have been created naturally by the human immune system.

As used herein, the term “epitope” includes any structural determinant capable of specific binding to an immunoglobulin or T-cell receptors. Epitopic determinants usually consist of chemically active surface groupings of molecules such as amino acids or sugar side chains, or combinations thereof and usually have specific three dimensional structural characteristics, as well as specific charge characteristics. Two antibodies are said to ‘bind the same epitope’ if one antibody is shown to compete with the second antibody in a competitive binding assay, by any of the methods well known to those of skill in the art.

An “isolated” antibody is one that has been identified and separated from a component of the cell that expressed it. Contaminant components of the cell are materials that would interfere with diagnostic or therapeutic uses of the antibody, and may include enzymes, hormones, and other proteinaceous or nonproteinaceous solutes. In preferred embodiments, the antibody is purified (1) to greater than 95% by weight of antibody as determined e.g. by the Lowry method, UV-Vis spectroscopy or by by SDS-Capillary Gel electrophoresis (for example on a Caliper LabChip GXII, GX 90 or Biorad Bioanalyzer device), and in further preferred embodiments more than 99% by weight, (2) to a degree sufficient to obtain at least 15 residues of N-terminal or internal amino acid sequence, or (3) to homogeneity by SDS-PAGE under reducing or nonreducing conditions using Coomassie blue or, preferably, silver stain. Isolated naturally occurring antibody includes the antibody in situ within recombinant cells since at least one component of the antibody's natural environment will not be present. Ordinarily, however, isolated antibody will be prepared by at least one purification step.

“Percent (%) sequence identity” with respect to a reference polynucleotide or polypeptide sequence, respectively, is defined as the percentage of nucleic acid or amino acid residues, respectively, in a candidate sequence that are identical with the nucleic acid or amino acid residues, respectively, in the reference polynucleotide or polypeptide sequence, respectively, after aligning the sequences and introducing gaps, if necessary, to achieve the maximum percent sequence identity. Conservative substitutions are not considered as part of the sequence identity. Preferred are un-gapped alignments. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

“FXa inhibitors comprising structure formula 1” are defined by compounds comprising a group of the formula 1

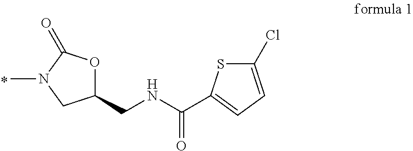

wherein * is the attachment site to the remaining part of the compound.

“FXa inhibitors comprising structure formula 2” are defined by compounds comprising a group of the formula 2

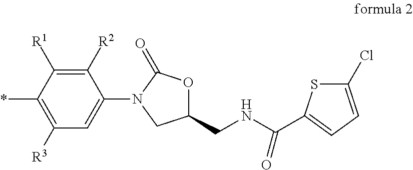

wherein
R¹ is hydrogen, R² is hydrogen and R³ is hydrogen,
or
R¹ is methyl, R² is hydrogen and R³ is methyl,
or
R¹ is hydrogen, R² is fluoro and R³ is hydrogen,
and
* is the attachment site to the remaining part of the compound.

“Neutralize”, “reverse”, “eliminate” or “normalize” the activity of coagulation inhibitors or similar phrases refer to inhibit or block the inhibitory or anticoagulant function of said inhibitor. Such phrases refer to partial inhibition or blocking of the function, as well as to inhibiting or blocking most or all of the activity of said inhibitor, in vitro and/or in vivo. In a preferred embodiment, the coagulation inhibitor is neutralized substantially meaning that its ability to inhibit said coagulation inhibitor, either directly or indirectly, is reduced by at least about 5%, 10%, 15%, 20%, 25%, 30%, 35%, 40%, 45%, 50%, 55%, 60%, 65%, 70%, 75%, 80%, 85, 90%, 95%, or 100%.

“Antibody mimetics” are Affibodies, Adnectins, Anticalins, DARPins, Avimers, Nanobodies (reviewed by Gebauer M. et al., Curr. Opinion in Chem. Biol. 2009; 13:245-255; Nuttall S. D. et al., Curr. Opinion in Pharmacology 2008; 8:608-617) and Aptamers (reviewed by Keefe A D., et al., Nat. Rev. Drug Discov. 2010; 9:537-550).

### Antibodies of the Invention

The present invention relates to the identification and use of antibodies and functional fragments thereof, or antibody mimetics suitable to neutralize the anti-coagulant activity of therapeutic inhibitors of coagulation in vitro and/or in vivo. In a preferred embodiment the in vitro inhibition is determined in a PT, aPTT, a Thrombin generation or a biochemical assay. In a preferred embodiment the in vivo inhibition is determined in a tail-bleeding experiment.

Another embodiment are antibodies and functional fragments thereof of the invention, or antibody mimetics binding to therapeutic inhibitors of coagulation.

In a preferred embodiment, the antibodies of the invention and functional fragments thereof or antibody mimetics bind to an anticoagulant and neutralizes the anticoagulant activity of said anticoagulant in vitro and/or in vivo.

In a preferred embodiment, the antibodies of the invention and functional fragments thereof, or antibody mimetics bind to an anticoagulant and neutralizes the anticoagulant activity of said first anticoagulant in vitro and/or in vivo and not neutralizes another anticoagulant as said first anticoagulant.

In a preferred embodiment, the antibodies of the invention and functional fragments thereof, or antibody mimetics bind specifically to an anticoagulant and specifically neutralizes the anticoagulant activity of said first anticoagulant in vitro and/or in vivo and not neutralizes another anticoagulant as said first anticoagulant.

In a further preferred embodiment the anticoagulant is a small molecule, preferably of a molecular weight of less than 5000 Da, less than 2500 Da and more preferred less than 1000 Da. Preferred anticoagulant are inhibitors of FXa or thrombin (dabigatran (Sorbera et al., Drugs of the Future 2005, 30(9):877-885 and references cited therein).

In a further preferred embodiment a FXa inhibitor is a compound comprising a group of the formula 1, apixaban (see WO2003/026652; Example 18), betrixaban (see U.S. Pat. No. 6,376,515 and U.S. Pat. No. 6,835,739), razaxaban (see WO1998/057951; Example 34), edoxaban (see US 2005 0020645; Example 192), otamixaban (Guertin et al., Current Medicinal Chemistry 2007, 14, 2471-2781 and references cited therein) or YM-150.

In a further preferred embodiment a compound comprising a group of the formula 1 is a compound comprising a group of the formula 2. In an even further preferred embodiment a compound comprising a group of the formula 2 is rivaroxaban, SATI (see WO 2008/155032 (Example 38)) and the compound of Example 1G. In an even more preferred embodiment a compound comprising a group of the formula 2 is rivaroxaban.

In a preferred embodiment, the antibodies of the invention or antigen-binding fragments thereof or antibody mimetics have a binding affinity (K_{D}) of less than 500 nM, preferably less than 250 nM, less than 100 nM, less than 50 nM, or more preferably less than 25 nM. The binding affinity is preferably determined by the method described in example 7.

In a preferred embodiment, the antibodies of the invention or antigen-binding fragments thereof or antibody mimetics neutralizes the anti-coagulant with half-maximal effective concentrations (EC50) in a biochemical assay inhibited with the respective anticoagulant of EC50 <2 μM, <1 μM, <0.5 μM or, preferably <0.01 μM. In a preferred embodiment, the antibodies of the invention or antigen-binding fragments thereof or antibody mimetics neutralizes the anti-coagulant with half-maximal effective concentrations (EC50) in a biochemical FXa-assay inhibited with rivaroxaban of EC50 <2 μM, <1 μM, <0.5 μM or, preferably <0.01 μM.

In a preferred embodiment, the antibodies of the invention or antigen-binding fragments thereof or antibody mimetics compete in binding to the anticoagulant with an antibody of table 1, preferably with antibody M14-G07, M18-G08, M18-G08-G or M18-G08-G-DKTHT. In a further embodiment, the above competing antibody or antigen-binding fragment thereof competes in binding to rivaroxaban with M18-G08-G-DKTHT.

In a further embodiment the antibody or antigen-binding fragment thereof competes in binding to rivaroxaban with M18-G08-G-DKTHT wherein binding of the antibody or antigen binding fragment thereof is mediated via a) a π-stacking of an amino acid residue at position 99 of the light chain to the chlorthiophene moiety of rivaroxaban, b) hydrophobic stacking of an amino acid residue at position 104 of the heavy chain to the chlorthiophene moiety of rivaroxaban, c) hydrogen bonding of an amino acid residue at position 50 (a hydrogen-bond donor amino acid) and 102 (in case of position 102 via the backbone amide of the polypeptide chain) of the heavy chain to the central amide of rivaroxaban, d) hydrogen bonding of a hydrogen-bond acceptor amino acid residue at position 102 of the heavy chain to the carbonyl oxygen of the oxazole of rivaroxaban, and e) π-stacking of an amino acid residue at position 33 of the heavy chain to the phenyl ring of rivaroxaban.

In another further embodiment the antibody or antigen-binding fragment thereof competes in binding to rivaroxaban with M18-G08-G-DKTHT wherein the the amino acid residue at position 99 of the light chain is selected from the group consisting of Trp, Phe and Tyr. In another further embodiment the amino acid residue at position 104 of the heavy chain is a hydrophobic amino acid, preferrably selected from the group consisting of Ala, Val, Leu, Ile, Met, and Phe. In another further embodiment the amino acid residue at position 50 is a hydrogen-bond donor amino acid residue and preferably selected from the group consisting Ser, Thr, Tyr, Trp, His, Asn and Gln. In another further embodiment the amino acid residue at position 102 of the heavy chain is a hydrogen-bond acceptor amino acid and preferably selected from the group consisting Ser, Thr, Tyr, Glu, Asp, Asn and Gln, In another further embodiment the amino acid residue at position 33 of the heavy chain is selected from the group consisting of Trp, Phe and Tyr.

In another further embodiment the antibody or antigen-binding fragment thereof competes in binding to rivaroxaban with M18-G08-G-DKTHT

wherein the amino acid residue at position 99 of the light chain is selected from the group consisting of Trp, Phe and Tyr,

the amino acid residue at position 104 of the heavy chain is a hydrophobic amino acid selected from the group consisting of Ala, Val, Leu, Ile, Met, and Phe, and the amino acid residue at position 102 of the heavy chain is a hydrogen-bond acceptor amino acid selected from the group consisting Ser, Thr, Tyr, Glu, Asp, Asn and Gln.

In another further embodiment the antibody or antigen-binding fragment thereof competes in binding to rivaroxaban with M18-G08-G-DKTHT

wherein the amino acid residue at position 99 of the light chain is Trp,

the amino acid residue at position 102 of the heavy chain is Thr or Asn, and

the amino acid residue at position 104 of the heavy chain is Leu.

In another embodiment, the above competing antibody or antigen-binding fragment competes in binding to rivaroxaban with M18-G08-G-DKTHT and has a variable light chain sequence comprising Asn at position 35, Tyr at position 37, Gln at position 90, Trp at position 99, and Phe at position 101 (numbering according to the amino acid positions of Fab M18-G08-G-DKTHT variable light chain) and a variable heavy hain sequence comprising Ser at position 31, Trp at position 33, Ser at position 35, Trp at position 47, Ser at position 50, Val at position 99, Trp at position 100, Arg at position 101, Asn at position 102, Tyr at position 103 and Leu at position 104 (numbering according to the amino acid positions of Fab M18-G08-G-DKTHT variable heavy chain).

In a further embodiment the aforementioned competing antibody is at least 90% identical to the Vh and Vl sequence of M18-G08-G, respectively.

The antibodies, antigen-binding antibody fragments, and variants of the antibodies and fragments of the invention are comprised of a light chain variable region and a heavy chain variable region. Variants of the antibodies or antigen-binding antibody fragments contemplated in the invention are molecules in which the binding activity of the antibody or antigen-binding antibody fragment for the antigen is maintained.

Throughout this document, reference is made to the following representative antibodies or functional fragments thereof of the invention: M14-G07, M18-G08, M18-G08-G and M18-G08-G-DKTHT. The respective sequences (SEQ ID NOs) are depicted in table 1.

TABLE 1
Antibodies and their respective sequences

| Antibody | Description | type | SEQ ID NO: |
|---|---|---|---|
| M18-G08-1 | Vh | PRT | SEQ ID NO: 1 |
| M18-G08-1 | Vl | PRT | SEQ ID NO: 2 |
| M18-G08-2 | Vh | PRT | SEQ ID NO: 3 |
| M18-G08-2 | Vl | PRT | SEQ ID NO: 4 |
| M18-G08-3 | Vh | PRT | SEQ ID NO: 5 |
| M18-G08-3 | Vl | PRT | SEQ ID NO: 6 |
| M18-G08-4 | Vh | PRT | SEQ ID NO: 7 |
| M18-G08-4 | Vl | PRT | SEQ ID NO: 8 |
| M18-G08-5 | Vh | PRT | SEQ ID NO: 9 |
| M18-G08-5 | Vl | PRT | SEQ ID NO: 10 |
| M18-G08-6 | Vh | PRT | SEQ ID NO: 11 |
| M18-G08-6 | Vl | PRT | SEQ ID NO: 12 |
| M18-G08-7 | Vh | PRT | SEQ ID NO: 13 |
| M18-G08-7 | Vl | PRT | SEQ ID NO: 14 |
| M18-G08-8 | Vh | PRT | SEQ ID NO: 15 |
| M18-G08-8 | Vl | PRT | SEQ ID NO: 16 |
| M18-G08-9 | Vh | PRT | SEQ ID NO: 17 |
| M18-G08-9 | Vl | PRT | SEQ ID NO: 18 |
| M18-G08-10 | Vh | PRT | SEQ ID NO: 19 |
| M18-G08-10 | Vl | PRT | SEQ ID NO: 20 |
| M18-G08-11 | Vh | PRT | SEQ ID NO: 21 |
| M18-G08-11 | Vl | PRT | SEQ ID NO: 22 |
| M18-G08-12 | Vh | PRT | SEQ ID NO: 23 |
| M18-G08-12 | Vl | PRT | SEQ ID NO: 24 |
| M18-G08-13 | Vh | PRT | SEQ ID NO: 25 |
| M18-G08-13 | Vl | PRT | SEQ ID NO: 26 |
| M18-G08-14 | Vh | PRT | SEQ ID NO: 27 |
| M18-G08-14 | Vl | PRT | SEQ ID NO: 28 |
| M18-G08-15 | Vh | PRT | SEQ ID NO: 29 |
| M18-G08-15 | Vl | PRT | SEQ ID NO: 30 |
| M18-G08-16 | Vh | PRT | SEQ ID NO: 31 |
| M18-G08-16 | Vl | PRT | SEQ ID NO: 32 |
| M18-G08-17 | Vh | PRT | SEQ ID NO: 33 |
| M18-G08-17 | Vl | PRT | SEQ ID NO: 34 |
| M18-G08-18 | Vh | PRT | SEQ ID NO: 35 |
| M18-G08-18 | Vl | PRT | SEQ ID NO: 36 |
| M18-G08-19 | Vh | PRT | SEQ ID NO: 37 |
| M18-G08-19 | Vl | PRT | SEQ ID NO: 38 |
| M18-G08-20 | Vh | PRT | SEQ ID NO: 39 |
| M18-G08-20 | Vl | PRT | SEQ ID NO: 40 |
| M18-G08-21 | Vh | PRT | SEQ ID NO: 41 |
| M18-G08-21 | Vl | PRT | SEQ ID NO: 42 |
| M18-G08-22 | Vh | PRT | SEQ ID NO: 43 |
| M18-G08-22 | Vl | PRT | SEQ ID NO: 44 |
| M18-G08-23 | Vh | PRT | SEQ ID NO: 45 |
| M18-G08-23 | Vl | PRT | SEQ ID NO: 46 |
| M18-G08-24 | Vh | PRT | SEQ ID NO: 47 |
| M18-G08-24 | Vl | PRT | SEQ ID NO: 48 |
| M18-G08-25 | Vh | PRT | SEQ ID NO: 49 |
| M18-G08-25 | Vl | PRT | SEQ ID NO: 50 |
| M18-G08-26 | Vh | PRT | SEQ ID NO: 51 |
| M18-G08-26 | Vl | PRT | SEQ ID NO: 52 |
| M18-G08-27 | Vh | PRT | SEQ ID NO: 53 |
| M18-G08-27 | Vl | PRT | SEQ ID NO: 54 |
| M18-G08-28 | Vh | PRT | SEQ ID NO: 55 |
| M18-G08-28 | Vl | PRT | SEQ ID NO: 56 |
| M18-G08-29 | Vh | PRT | SEQ ID NO: 57 |
| M18-G08-29 | Vl | PRT | SEQ ID NO: 58 |
| M18-G08-30 | Vh | PRT | SEQ ID NO: 59 |
| M18-G08-30 | Vl | PRT | SEQ ID NO: 60 |
| M18-G08-31 | Vh | PRT | SEQ ID NO: 61 |
| M18-G08-31 | Vl | PRT | SEQ ID NO: 62 |
| M18-G08-32 | Vh | PRT | SEQ ID NO: 63 |
| M18-G08-32 | Vl | PRT | SEQ ID NO: 64 |
| M18-G08-33 | Vh | PRT | SEQ ID NO: 65 |
| M18-G08-33 | Vl | PRT | SEQ ID NO: 66 |
| M18-G08-34 | Vh | PRT | SEQ ID NO: 67 |
| M18-G08-34 | Vl | PRT | SEQ ID NO: 68 |
| M18-G08-35 | Vh | PRT | SEQ ID NO: 69 |
| M18-G08-35 | Vl | PRT | SEQ ID NO: 70 |
| M18-G08-36 | Vh | PRT | SEQ ID NO: 71 |
| M18-G08-36 | Vl | PRT | SEQ ID NO: 72 |
| M18-G08-37 | Vh | PRT | SEQ ID NO: 73 |
| M18-G08-37 | Vl | PRT | SEQ ID NO: 74 |
| M18-G08-38 | Vh | PRT | SEQ ID NO: 75 |
| M18-G08-38 | Vl | PRT | SEQ ID NO: 76 |
| M18-G08-39 | Vh | PRT | SEQ ID NO: 77 |
| M18-G08-39 | Vl | PRT | SEQ ID NO: 78 |
| M18-G08-40 | Vh | PRT | SEQ ID NO: 79 |
| M18-G08-40 | Vl | PRT | SEQ ID NO: 80 |
| M18-G08-41 | Vh | PRT | SEQ ID NO: 81 |
| M18-G08-41 | Vl | PRT | SEQ ID NO: 82 |
| M18-G08-42 | Vh | PRT | SEQ ID NO: 83 |
| M18-G08-42 | Vl | PRT | SEQ ID NO: 84 |
| M18-G08-43 | Vh | PRT | SEQ ID NO: 85 |
| M18-G08-43 | Vl | PRT | SEQ ID NO: 86 |
| M18-G08-44 | Vh | PRT | SEQ ID NO: 87 |
| M18-G08-44 | Vl | PRT | SEQ ID NO: 88 |
| M18-G08-45 | Vh | PRT | SEQ ID NO: 89 |
| M18-G08-45 | Vl | PRT | SEQ ID NO: 90 |
| M18-G08-46 | Vh | PRT | SEQ ID NO: 91 |
| M18-G08-46 | Vl | PRT | SEQ ID NO: 92 |
| M18-G08-47 | Vh | PRT | SEQ ID NO: 93 |
| M18-G08-47 | Vl | PRT | SEQ ID NO: 94 |
| M18-G08-48 | Vh | PRT | SEQ ID NO: 95 |
| M18-G08-48 | Vl | PRT | SEQ ID NO: 96 |
| M18-G08-49 | Vh | PRT | SEQ ID NO: 97 |
| M18-G08-49 | Vl | PRT | SEQ ID NO: 98 |
| M18-G08-50 | Vh | PRT | SEQ ID NO: 99 |
| M18-G08-50 | Vl | PRT | SEQ ID NO: 100 |
| M18-G08-51 | Vh | PRT | SEQ ID NO: 101 |
| M18-G08-51 | Vl | PRT | SEQ ID NO: 102 |
| M18-G08-52 | Vh | PRT | SEQ ID NO: 103 |
| M18-G08-52 | Vl | PRT | SEQ ID NO: 104 |
| M18-G08-53 | Vh | PRT | SEQ ID NO: 105 |
| M18-G08-53 | Vl | PRT | SEQ ID NO: 106 |
| M18-G08-54 | Vh | PRT | SEQ ID NO: 107 |
| M18-G08-54 | Vl | PRT | SEQ ID NO: 108 |
| M18-G08-55 | Vh | PRT | SEQ ID NO: 109 |
| M18-G08-55 | Vl | PRT | SEQ ID NO: 110 |
| M18-G08-56 | Vh | PRT | SEQ ID NO: 111 |
| M18-G08-56 | Vl | PRT | SEQ ID NO: 112 |
| M18-G08-57 | Vh | PRT | SEQ ID NO: 113 |
| M18-G08-57 | Vl | PRT | SEQ ID NO: 114 |
| M18-G08-58 | Vh | PRT | SEQ ID NO: 115 |
| M18-G08-58 | Vl | PRT | SEQ ID NO: 116 |
| M18-G08-G | Vh | PRT | SEQ ID NO: 117 |
| M18-G08-G | Vl | PRT | SEQ ID NO: 118 |
| M18-G08-62 | Vh | PRT | SEQ ID NO: 119 |
| M18-G08-62 | Vl | PRT | SEQ ID NO: 120 |
| M18-G08-63 | Vh | PRT | SEQ ID NO: 121 |
| M18-G08-63 | Vl | PRT | SEQ ID NO: 122 |
| M18-G08-64 | Vh | PRT | SEQ ID NO: 123 |
| M18-G08-64 | Vl | PRT | SEQ ID NO: 124 |
| M18-G08-65 | Vh | PRT | SEQ ID NO: 125 |
| M18-G08-65 | Vl | PRT | SEQ ID NO: 126 |
| M14-G07-1 | Vh | PRT | SEQ ID NO: 127 |
| M14-G07-1 | Vl | PRT | SEQ ID NO: 128 |
| M14-G07-2 | Vh | PRT | SEQ ID NO: 129 |
| M14-G07-2 | Vl | PRT | SEQ ID NO: 130 |
| M14-G07-3 | Vh | PRT | SEQ ID NO: 131 |
| M14-G07-3 | Vl | PRT | SEQ ID NO: 132 |
| M14-G07-4 | Vh | PRT | SEQ ID NO: 133 |
| M14-G07-4 | Vl | PRT | SEQ ID NO: 134 |
| M14-G07-5 | Vh | PRT | SEQ ID NO: 135 |
| M14-G07-5 | Vl | PRT | SEQ ID NO: 136 |
| M14-G07-6 | Vh | PRT | SEQ ID NO: 137 |
| M14-G07-6 | Vl | PRT | SEQ ID NO: 138 |
| M14-G07-7 | Vh | PRT | SEQ ID NO: 139 |
| M14-G07-7 | Vl | PRT | SEQ ID NO: 140 |
| M14-G07-8 | Vh | PRT | SEQ ID NO: 141 |
| M14-G07-8 | Vl | PRT | SEQ ID NO: 142 |
| M14-G07-9 | Vh | PRT | SEQ ID NO: 143 |
| M14-G07-9 | Vl | PRT | SEQ ID NO: 144 |
| M14-G07-10 | Vh | PRT | SEQ ID NO: 145 |
| M14-G07-10 | Vl | PRT | SEQ ID NO: 146 |
| M14-G07-11 | Vh | PRT | SEQ ID NO: 147 |
| M14-G07-11 | Vl | PRT | SEQ ID NO: 148 |
| M14-G07-12 | Vh | PRT | SEQ ID NO: 149 |
| M14-G07-12 | Vl | PRT | SEQ ID NO: 150 |
| M14-G07-13 | Vh | PRT | SEQ ID NO: 151 |
| M14-G07-13 | Vl | PRT | SEQ ID NO: 152 |
| M14-G07-14 | Vh | PRT | SEQ ID NO: 153 |
| M14-G07-14 | Vl | PRT | SEQ ID NO: 154 |
| M14-G07-15 | Vh | PRT | SEQ ID NO: 155 |
| M14-G07-15 | Vl | PRT | SEQ ID NO: 156 |
| M14-G07-16 | Vh | PRT | SEQ ID NO: 157 |
| M14-G07-16 | Vl | PRT | SEQ ID NO: 158 |
| M14-G07-17 | Vh | PRT | SEQ ID NO: 159 |
| M14-G07-17 | Vl | PRT | SEQ ID NO: 160 |
| M14-G07-18 | Vh | PRT | SEQ ID NO: 161 |
| M14-G07-18 | Vl | PRT | SEQ ID NO: 162 |
| M14-G07-19 | Vh | PRT | SEQ ID NO: 163 |
| M14-G07-19 | Vl | PRT | SEQ ID NO: 164 |
| M14-G07-20 | Vh | PRT | SEQ ID NO: 165 |
| M14-G07-20 | Vl | PRT | SEQ ID NO: 166 |
| M14-G07-21 | Vh | PRT | SEQ ID NO: 167 |
| M14-G07-21 | Vl | PRT | SEQ ID NO: 168 |
| M14-G07-22 | Vh | PRT | SEQ ID NO: 169 |
| M14-G07-22 | Vl | PRT | SEQ ID NO: 170 |
| M14-G07-23 | Vh | PRT | SEQ ID NO: 171 |
| M14-G07-23 | Vl | PRT | SEQ ID NO: 172 |
| M14-G07-24 | Vh | PRT | SEQ ID NO: 173 |
| M14-G07-24 | Vl | PRT | SEQ ID NO: 174 |
| M14-G07-25 | Vh | PRT | SEQ ID NO: 175 |
| M14-G07-25 | Vl | PRT | SEQ ID NO: 176 |
| M14-G07-26 | Vh | PRT | SEQ ID NO: 177 |
| M14-G07-26 | Vl | PRT | SEQ ID NO: 178 |
| M14-G07-27 | Vh | PRT | SEQ ID NO: 179 |
| M14-G07-27 | Vl | PRT | SEQ ID NO: 180 |
| M14-G07-28 | Vh | PRT | SEQ ID NO: 181 |
| M14-G07-28 | Vl | PRT | SEQ ID NO: 182 |
| M14-G07-29 | Vh | PRT | SEQ ID NO: 183 |
| M14-G07-29 | Vl | PRT | SEQ ID NO: 184 |
| M14-G07-30 | Vh | PRT | SEQ ID NO: 185 |
| M14-G07-30 | Vl | PRT | SEQ ID NO: 186 |
| M14-G07-31 | Vh | PRT | SEQ ID NO: 187 |
| M14-G07-31 | Vl | PRT | SEQ ID NO: 188 |
| M14-G07-32 | Vh | PRT | SEQ ID NO: 189 |
| M14-G07-32 | Vl | PRT | SEQ ID NO: 190 |
| M14-G07-33 | Vh | PRT | SEQ ID NO: 191 |
| M14-G07-33 | Vl | PRT | SEQ ID NO: 192 |
| M14-G07-34 | Vh | PRT | SEQ ID NO: 193 |
| M14-G07-34 | Vl | PRT | SEQ ID NO: 194 |
| M14-G07-35 | Vh | PRT | SEQ ID NO: 195 |
| M14-G07-35 | Vl | PRT | SEQ ID NO: 196 |
| M14-G07-36 | Vh | PRT | SEQ ID NO: 197 |
| M14-G07-36 | Vl | PRT | SEQ ID NO: 198 |
| M14-G07-37 | Vh | PRT | SEQ ID NO: 199 |
| M14-G07-37 | Vl | PRT | SEQ ID NO: 200 |
| M14-G07-38 | Vh | PRT | SEQ ID NO: 201 |
| M14-G07-38 | Vl | PRT | SEQ ID NO: 202 |
| M14-G07-39 | Vh | PRT | SEQ ID NO: 203 |
| M14-G07-39 | Vl | PRT | SEQ ID NO: 204 |
| M14-G07-40 | Vh | PRT | SEQ ID NO: 205 |
| M14-G07-40 | Vl | PRT | SEQ ID NO: 206 |
| M14-G07 | Vh | PRT | SEQ ID NO: 207 |
| M14-G07 | Vl | PRT | SEQ ID NO: 208 |
| M15-B07 | Vh | PRT | SEQ ID NO: 209 |
| M15-B07 | Vl | PRT | SEQ ID NO: 210 |
| M16-A03 | Vh | PRT | SEQ ID NO: 211 |
| M16-A03 | Vl | PRT | SEQ ID NO: 212 |
| M16-D05 | Vh | PRT | SEQ ID NO: 213 |
| M16-D05 | Vl | PRT | SEQ ID NO: 214 |
| M18-A10 | Vh | PRT | SEQ ID NO: 215 |
| M18-A10 | Vl | PRT | SEQ ID NO: 216 |
| M18-G08 | Vh | PRT | SEQ ID NO: 217 |
| M18-G08 | Vl | PRT | SEQ ID NO: 218 |
| M25-E05 | Vh | PRT | SEQ ID NO: 219 |
| M25-E05 | Vl | PRT | SEQ ID NO: 220 |
| M14-G07 | H-CDR1 | PRT | SEQ ID NO: 221 |
| M14-G07 | H-CDR2 | PRT | SEQ ID NO: 222 |
| M14-G07 | H-CDR3 | PRT | SEQ ID NO: 223 |
| M14-G07 | L-CDR1 | PRT | SEQ ID NO: 224 |
| M14-G07 | L-CDR2 | PRT | SEQ ID NO: 225 |
| M14-G07 | L-CDR3 | PRT | SEQ ID NO: 226 |
| M15-B07 | H-CDR1 | PRT | SEQ ID NO: 227 |
| M15-B07 | H-CDR2 | PRT | SEQ ID NO: 228 |
| M15-B07 | H-CDR3 | PRT | SEQ ID NO: 229 |
| M15-B07 | L-CDR1 | PRT | SEQ ID NO: 230 |
| M15-B07 | L-CDR2 | PRT | SEQ ID NO: 231 |
| M15-B07 | L-CDR3 | PRT | SEQ ID NO: 232 |
| M16-A03 | H-CDR1 | PRT | SEQ ID NO: 233 |
| M16-A03 | H-CDR2 | PRT | SEQ ID NO: 234 |
| M16-A03 | H-CDR3 | PRT | SEQ ID NO: 235 |
| M16-A03 | L-CDR1 | PRT | SEQ ID NO: 236 |
| M16-A03 | L-CDR2 | PRT | SEQ ID NO: 237 |
| M16-A03 | L-CDR3 | PRT | SEQ ID NO: 238 |
| M16-D05 | H-CDR1 | PRT | SEQ ID NO: 239 |
| M16-D05 | H-CDR2 | PRT | SEQ ID NO: 240 |
| M16-D05 | H-CDR3 | PRT | SEQ ID NO: 241 |
| M16-D05 | L-CDR1 | PRT | SEQ ID NO: 242 |
| M16-D05 | L-CDR2 | PRT | SEQ ID NO: 243 |
| M16-D05 | L-CDR3 | PRT | SEQ ID NO: 244 |
| M18-A10 | H-CDR1 | PRT | SEQ ID NO: 245 |
| M18-A10 | H-CDR2 | PRT | SEQ ID NO: 246 |
| M18-A10 | H-CDR3 | PRT | SEQ ID NO: 247 |
| M18-A10 | L-CDR1 | PRT | SEQ ID NO: 248 |
| M18-A10 | L-CDR2 | PRT | SEQ ID NO: 249 |
| M18-A10 | L-CDR3 | PRT | SEQ ID NO: 250 |
| M18-G08 | H-CDR1 | PRT | SEQ ID NO: 251 |
| M18-G08 | H-CDR2 | PRT | SEQ ID NO: 252 |
| M18-G08 | H-CDR3 | PRT | SEQ ID NO: 253 |
| M18-G08 | L-CDR1 | PRT | SEQ ID NO: 254 |
| M18-G08 | L-CDR2 | PRT | SEQ ID NO: 255 |
| M18-G08 | L-CDR3 | PRT | SEQ ID NO: 256 |
| M25-E05 | H-CDR1 | PRT | SEQ ID NO: 257 |
| M25-E05 | H-CDR2 | PRT | SEQ ID NO: 258 |
| M25-E05 | H-CDR3 | PRT | SEQ ID NO: 259 |
| M25-E05 | L-CDR1 | PRT | SEQ ID NO: 260 |
| M25-E05 | L-CDR2 | PRT | SEQ ID NO: 261 |
| M25-E05 | L-CDR3 | PRT | SEQ ID NO: 262 |
| M18-G08-G | H-CDR1 | PRT | SEQ ID NO: 263 |
| M18-G08-G | H-CDR2 | PRT | SEQ ID NO: 264 |
| M18-G08-G | H-CDR3 | PRT | SEQ ID NO: 265 |
| M18-G08-G | L-CDR1 | PRT | SEQ ID NO: 266 |
| M18-G08-G | L-CDR2 | PRT | SEQ ID NO: 267 |
| M18-G08-G | L-CDR3 | PRT | SEQ ID NO: 268 |
| M18-G08-1 | Vh | DNA | SEQ ID NO: 269 |
| M18-G08-1 | Vl | DNA | SEQ ID NO: 270 |
| M18-G08-2 | Vh | DNA | SEQ ID NO: 271 |
| M18-G08-2 | Vl | DNA | SEQ ID NO: 272 |
| M18-G08-3 | Vh | DNA | SEQ ID NO: 273 |
| M18-G08-3 | Vl | DNA | SEQ ID NO: 274 |
| M18-G08-4 | Vh | DNA | SEQ ID NO: 275 |
| M18-G08-4 | Vl | DNA | SEQ ID NO: 276 |
| M18-G08-5 | Vh | DNA | SEQ ID NO: 277 |
| M18-G08-5 | Vl | DNA | SEQ ID NO: 278 |
| M18-G08-6 | Vh | DNA | SEQ ID NO: 279 |
| M18-G08-6 | Vl | DNA | SEQ ID NO: 280 |
| M18-G08-7 | Vh | DNA | SEQ ID NO: 281 |
| M18-G08-7 | Vl | DNA | SEQ ID NO: 282 |
| M18-G08-8 | Vh | DNA | SEQ ID NO: 283 |
| M18-G08-8 | Vl | DNA | SEQ ID NO: 284 |
| M18-G08-9 | Vh | DNA | SEQ ID NO: 285 |
| M18-G08-9 | Vl | DNA | SEQ ID NO: 286 |
| M18-G08-10 | Vh | DNA | SEQ ID NO: 287 |
| M18-G08-10 | Vl | DNA | SEQ ID NO: 288 |
| M18-G08-11 | Vh | DNA | SEQ ID NO: 289 |
| M18-G08-11 | Vl | DNA | SEQ ID NO: 290 |
| M18-G08-12 | Vh | DNA | SEQ ID NO: 291 |
| M18-G08-12 | Vl | DNA | SEQ ID NO: 292 |
| M18-G08-13 | Vh | DNA | SEQ ID NO: 293 |
| M18-G08-13 | Vl | DNA | SEQ ID NO: 294 |
| M18-G08-14 | Vh | DNA | SEQ ID NO: 295 |
| M18-G08-14 | Vl | DNA | SEQ ID NO: 296 |
| M18-G08-15 | Vh | DNA | SEQ ID NO: 297 |
| M18-G08-15 | Vl | DNA | SEQ ID NO: 298 |
| M18-G08-16 | Vh | DNA | SEQ ID NO: 299 |
| M18-G08-16 | Vl | DNA | SEQ ID NO: 300 |
| M18-G08-17 | Vh | DNA | SEQ ID NO: 301 |
| M18-G08-17 | Vl | DNA | SEQ ID NO: 302 |
| M18-G08-18 | Vh | DNA | SEQ ID NO: 303 |
| M18-G08-18 | Vl | DNA | SEQ ID NO: 304 |
| M18-G08-19 | Vh | DNA | SEQ ID NO: 305 |
| M18-G08-19 | Vl | DNA | SEQ ID NO: 306 |
| M18-G08-20 | Vh | DNA | SEQ ID NO: 307 |
| M18-G08-20 | Vl | DNA | SEQ ID NO: 308 |
| M18-G08-21 | Vh | DNA | SEQ ID NO: 309 |
| M18-G08-21 | Vl | DNA | SEQ ID NO: 310 |
| M18-G08-22 | Vh | DNA | SEQ ID NO: 311 |
| M18-G08-22 | Vl | DNA | SEQ ID NO: 312 |
| M18-G08-23 | Vh | DNA | SEQ ID NO: 313 |
| M18-G08-23 | Vl | DNA | SEQ ID NO: 314 |
| M18-G08-24 | Vh | DNA | SEQ ID NO: 315 |
| M18-G08-24 | Vl | DNA | SEQ ID NO: 316 |
| M18-G08-25 | Vh | DNA | SEQ ID NO: 317 |
| M18-G08-25 | Vl | DNA | SEQ ID NO: 318 |
| M18-G08-26 | Vh | DNA | SEQ ID NO: 319 |
| M18-G08-26 | Vl | DNA | SEQ ID NO: 320 |
| M18-G08-27 | Vh | DNA | SEQ ID NO: 321 |
| M18-G08-27 | Vl | DNA | SEQ ID NO: 322 |
| M18-G08-28 | Vh | DNA | SEQ ID NO: 323 |
| M18-G08-28 | Vl | DNA | SEQ ID NO: 324 |
| M18-G08-29 | Vh | DNA | SEQ ID NO: 325 |
| M18-G08-29 | Vl | DNA | SEQ ID NO: 326 |
| M18-G08-30 | Vh | DNA | SEQ ID NO: 327 |
| M18-G08-30 | Vl | DNA | SEQ ID NO: 328 |
| M18-G08-31 | Vh | DNA | SEQ ID NO: 329 |
| M18-G08-31 | Vl | DNA | SEQ ID NO: 330 |
| M18-G08-32 | Vh | DNA | SEQ ID NO: 331 |
| M18-G08-32 | Vl | DNA | SEQ ID NO: 332 |
| M18-G08-33 | Vh | DNA | SEQ ID NO: 333 |
| M18-G08-33 | Vl | DNA | SEQ ID NO: 334 |
| M18-G08-34 | Vh | DNA | SEQ ID NO: 335 |
| M18-G08-34 | Vl | DNA | SEQ ID NO: 336 |
| M18-G08-35 | Vh | DNA | SEQ ID NO: 337 |
| M18-G08-35 | Vl | DNA | SEQ ID NO: 338 |
| M18-G08-36 | Vh | DNA | SEQ ID NO: 339 |
| M18-G08-36 | Vl | DNA | SEQ ID NO: 340 |
| M18-G08-37 | Vh | DNA | SEQ ID NO: 341 |
| M18-G08-37 | Vl | DNA | SEQ ID NO: 342 |
| M18-G08-38 | Vh | DNA | SEQ ID NO: 343 |
| M18-G08-38 | Vl | DNA | SEQ ID NO: 344 |
| M18-G08-39 | Vh | DNA | SEQ ID NO: 345 |
| M18-G08-39 | Vl | DNA | SEQ ID NO: 346 |
| M18-G08-40 | Vh | DNA | SEQ ID NO: 347 |
| M18-G08-40 | Vl | DNA | SEQ ID NO: 348 |
| M18-G08-41 | Vh | DNA | SEQ ID NO: 349 |
| M18-G08-41 | Vl | DNA | SEQ ID NO: 350 |
| M18-G08-42 | Vh | DNA | SEQ ID NO: 351 |
| M18-G08-42 | Vl | DNA | SEQ ID NO: 352 |
| M18-G08-43 | Vh | DNA | SEQ ID NO: 353 |
| M18-G08-43 | Vl | DNA | SEQ ID NO: 354 |
| M18-G08-44 | Vh | DNA | SEQ ID NO: 355 |
| M18-G08-44 | Vl | DNA | SEQ ID NO: 356 |
| M18-G08-45 | Vh | DNA | SEQ ID NO: 357 |
| M18-G08-45 | Vl | DNA | SEQ ID NO: 358 |
| M18-G08-46 | Vh | DNA | SEQ ID NO: 359 |
| M18-G08-46 | Vl | DNA | SEQ ID NO: 360 |
| M18-G08-47 | Vh | DNA | SEQ ID NO: 361 |
| M18-G08-47 | Vl | DNA | SEQ ID NO: 362 |
| M18-G08-48 | Vh | DNA | SEQ ID NO: 363 |
| M18-G08-48 | Vl | DNA | SEQ ID NO: 364 |
| M18-G08-49 | Vh | DNA | SEQ ID NO: 365 |
| M18-G08-49 | Vl | DNA | SEQ ID NO: 366 |
| M18-G08-50 | Vh | DNA | SEQ ID NO: 367 |
| M18-G08-50 | Vl | DNA | SEQ ID NO: 368 |
| M18-G08-51 | Vh | DNA | SEQ ID NO: 369 |
| M18-G08-51 | Vl | DNA | SEQ ID NO: 370 |
| M18-G08-52 | Vh | DNA | SEQ ID NO: 371 |
| M18-G08-52 | Vl | DNA | SEQ ID NO: 372 |
| M18-G08-53 | Vh | DNA | SEQ ID NO: 373 |
| M18-G08-53 | Vl | DNA | SEQ ID NO: 374 |
| M18-G08-54 | Vh | DNA | SEQ ID NO: 375 |
| M18-G08-54 | Vl | DNA | SEQ ID NO: 376 |
| M18-G08-55 | Vh | DNA | SEQ ID NO: 377 |
| M18-G08-55 | Vl | DNA | SEQ ID NO: 378 |
| M18-G08-56 | Vh | DNA | SEQ ID NO: 379 |
| M18-G08-56 | Vl | DNA | SEQ ID NO: 380 |
| M18-G08-57 | Vh | DNA | SEQ ID NO: 381 |
| M18-G08-57 | Vl | DNA | SEQ ID NO: 382 |
| M18-G08-58 | Vh | DNA | SEQ ID NO: 383 |
| M18-G08-58 | Vl | DNA | SEQ ID NO: 384 |
| M18-G08-G | Vh | DNA | SEQ ID NO: 385 |
| M18-G08-G | Vl | DNA | SEQ ID NO: 386 |
| M18-G08-62 | Vh | DNA | SEQ ID NO: 387 |
| M18-G08-62 | Vl | DNA | SEQ ID NO: 388 |
| M18-G08-63 | Vh | DNA | SEQ ID NO: 389 |
| M18-G08-63 | Vl | DNA | SEQ ID NO: 390 |
| M18-G08-64 | Vh | DNA | SEQ ID NO: 391 |
| M18-G08-64 | Vl | DNA | SEQ ID NO: 392 |
| M18-G08-65 | Vh | DNA | SEQ ID NO: 393 |
| M18-G08-65 | Vl | DNA | SEQ ID NO: 394 |
| M14-G07-1 | Vh | DNA | SEQ ID NO: 395 |
| M14-G07-1 | Vl | DNA | SEQ ID NO: 396 |
| M14-G07-2 | Vh | DNA | SEQ ID NO: 397 |
| M14-G07-2 | Vl | DNA | SEQ ID NO: 398 |
| M14-G07-3 | Vh | DNA | SEQ ID NO: 399 |
| M14-G07-3 | Vl | DNA | SEQ ID NO: 400 |
| M14-G07-4 | Vh | DNA | SEQ ID NO: 401 |
| M14-G07-4 | Vl | DNA | SEQ ID NO: 402 |
| M14-G07-5 | Vh | DNA | SEQ ID NO: 403 |
| M14-G07-5 | Vl | DNA | SEQ ID NO: 404 |
| M14-G07-6 | Vh | DNA | SEQ ID NO: 405 |
| M14-G07-6 | Vl | DNA | SEQ ID NO: 406 |
| M14-G07-7 | Vh | DNA | SEQ ID NO: 407 |
| M14-G07-7 | Vl | DNA | SEQ ID NO: 408 |
| M14-G07-8 | Vh | DNA | SEQ ID NO: 409 |
| M14-G07-8 | Vl | DNA | SEQ ID NO: 410 |
| M14-G07-9 | Vh | DNA | SEQ ID NO: 411 |
| M14-G07-9 | Vl | DNA | SEQ ID NO: 412 |
| M14-G07-10 | Vh | DNA | SEQ ID NO: 413 |
| M14-G07-10 | Vl | DNA | SEQ ID NO: 414 |
| M14-G07-11 | Vh | DNA | SEQ ID NO: 415 |
| M14-G07-11 | Vl | DNA | SEQ ID NO: 416 |
| M14-G07-12 | Vh | DNA | SEQ ID NO: 417 |
| M14-G07-12 | Vl | DNA | SEQ ID NO: 418 |
| M14-G07-13 | Vh | DNA | SEQ ID NO: 419 |
| M14-G07-13 | Vl | DNA | SEQ ID NO: 420 |
| M14-G07-14 | Vh | DNA | SEQ ID NO: 421 |
| M14-G07-14 | Vl | DNA | SEQ ID NO: 422 |
| M14-G07-15 | Vh | DNA | SEQ ID NO: 423 |
| M14-G07-15 | Vl | DNA | SEQ ID NO: 424 |
| M14-G07-16 | Vh | DNA | SEQ ID NO: 425 |
| M14-G07-16 | Vl | DNA | SEQ ID NO: 426 |
| M14-G07-17 | Vh | DNA | SEQ ID NO: 427 |
| M14-G07-17 | Vl | DNA | SEQ ID NO: 428 |
| M14-G07-18 | Vh | DNA | SEQ ID NO: 429 |
| M14-G07-18 | Vl | DNA | SEQ ID NO: 430 |
| M14-G07-19 | Vh | DNA | SEQ ID NO: 431 |
| M14-G07-19 | Vl | DNA | SEQ ID NO: 432 |
| M14-G07-20 | Vh | DNA | SEQ ID NO: 433 |
| M14-G07-20 | Vl | DNA | SEQ ID NO: 434 |
| M14-G07-21 | Vh | DNA | SEQ ID NO: 435 |
| M14-G07-21 | Vl | DNA | SEQ ID NO: 436 |
| M14-G07-22 | Vh | DNA | SEQ ID NO: 437 |
| M14-G07-22 | Vl | DNA | SEQ ID NO: 438 |
| M14-G07-23 | Vh | DNA | SEQ ID NO: 439 |
| M14-G07-23 | Vl | DNA | SEQ ID NO: 440 |
| M14-G07-24 | Vh | DNA | SEQ ID NO: 441 |
| M14-G07-24 | Vl | DNA | SEQ ID NO: 442 |
| M14-G07-25 | Vh | DNA | SEQ ID NO: 443 |
| M14-G07-25 | Vl | DNA | SEQ ID NO: 444 |
| M14-G07-26 | Vh | DNA | SEQ ID NO: 445 |
| M14-G07-26 | Vl | DNA | SEQ ID NO: 446 |
| M14-G07-27 | Vh | DNA | SEQ ID NO: 447 |
| M14-G07-27 | Vl | DNA | SEQ ID NO: 448 |
| M14-G07-28 | Vh | DNA | SEQ ID NO: 449 |
| M14-G07-28 | Vl | DNA | SEQ ID NO: 450 |
| M14-G07-29 | Vh | DNA | SEQ ID NO: 451 |
| M14-G07-29 | Vl | DNA | SEQ ID NO: 452 |
| M14-G07-30 | Vh | DNA | SEQ ID NO: 453 |
| M14-G07-30 | Vl | DNA | SEQ ID NO: 454 |
| M14-G07-31 | Vh | DNA | SEQ ID NO: 455 |
| M14-G07-31 | Vl | DNA | SEQ ID NO: 456 |
| M14-G07-32 | Vh | DNA | SEQ ID NO: 457 |
| M14-G07-32 | Vl | DNA | SEQ ID NO: 458 |
| M14-G07-33 | Vh | DNA | SEQ ID NO: 459 |
| M14-G07-33 | Vl | DNA | SEQ ID NO: 460 |
| M14-G07-34 | Vh | DNA | SEQ ID NO: 461 |
| M14-G07-34 | Vl | DNA | SEQ ID NO: 462 |
| M14-G07-35 | Vh | DNA | SEQ ID NO: 463 |
| M14-G07-35 | Vl | DNA | SEQ ID NO: 464 |
| M14-G07-36 | Vh | DNA | SEQ ID NO: 465 |
| M14-G07-36 | Vl | DNA | SEQ ID NO: 466 |
| M14-G07-37 | Vh | DNA | SEQ ID NO: 467 |
| M14-G07-37 | Vl | DNA | SEQ ID NO: 468 |
| M14-G07-38 | Vh | DNA | SEQ ID NO: 469 |
| M14-G07-38 | Vl | DNA | SEQ ID NO: 470 |
| M14-G07-39 | Vh | DNA | SEQ ID NO: 471 |
| M14-G07-39 | Vl | DNA | SEQ ID NO: 472 |
| M14-G07-40 | Vh | DNA | SEQ ID NO: 473 |
| M14-G07-40 | Vl | DNA | SEQ ID NO: 474 |
| M14-G07 | Vh | DNA | SEQ ID NO: 475 |
| M14-G07 | Vl | DNA | SEQ ID NO: 476 |
| M15-B07 | Vh | DNA | SEQ ID NO: 477 |
| M15-B07 | Vl | DNA | SEQ ID NO: 478 |
| M16-A03 | Vh | DNA | SEQ ID NO: 479 |
| M16-A03 | Vl | DNA | SEQ ID NO: 480 |
| M16-D05 | Vh | DNA | SEQ ID NO: 481 |
| M16-D05 | Vl | DNA | SEQ ID NO: 482 |
| M18-A10 | Vh | DNA | SEQ ID NO: 483 |
| M18-A10 | Vl | DNA | SEQ ID NO: 484 |
| M18-G08 | Vh | DNA | SEQ ID NO: 485 |
| M18-G08 | Vl | DNA | SEQ ID NO: 486 |
| M25-E05 | Vh | DNA | SEQ ID NO: 487 |
| M25-E05 | Vl | DNA | SEQ ID NO: 488 |
| M18-G08-G- | HC | PRT | SEQ ID NO: 489 |
| DKTHT | | | |
| M18-G08-G- | LC | PRT | SEQ ID NO: 490 |
| DKTHT | | | |
| M18-G08-G- | HC | DNA | SEQ ID NO: 491 |
| DKTHT | | | |
| M18-G08-G- | LC | DNA | SEQ ID NO: 492 |
| DKTHT | | | |
| M18-G08-DKTHT | HC | PRT | SEQ ID NO: 493 |
| M18-G08-DKTHT | LC | PRT | SEQ ID NO: 494 |
| M18-G08-DKTHT | HC | DNA | SEQ ID NO: 495 |
| M18-G08-DKTHT | LC | DNA | SEQ ID NO: 496 |

In a further preferred embodiment the antibodies of the invention or antigen-binding fragments thereof comprise heavy or light chain CDR sequences which are at least 50%, 55%, 60% 70%, 80%, 90%, or 95% identical to at least one, preferably corresponding, CDR sequence as depicted in table 1, or which comprise variable heavy or light chain sequences which are at least 50%, 60%, 70%, 80%, 90%, 92% or 95% identical to a VH or VL sequence depicted in table 1, respectively.

In a further preferred embodiment the antibodies of the invention or antigen-binding fragments thereof comprise heavy and/or light chain CDR sequences which are at least 50%, 55%, 60% 70%, 80%, 90%, or 95% identical to at least one, preferably corresponding, CDR sequence of the antibodies M14-G07, M18-G08, M18-G08-G or M18-G08-G-DKTHT, respectively.

In a further preferred embodiment the antibodies of the invention or antigen-binding fragments thereof comprise heavy and/or light chain CDR sequences which are at least 50%, 55%, 60% 70%, 80%, 90%, or 95% identical to the, preferably corresponding, heavy and/or light chain CDR sequences of the antibodies M14-G07, M18-G08, M18-G08-G or M18-G08-G-DKTHT, respectively.

In a further preferred embodiment the antibodies of the invention or antigen-binding fragments thereof comprise heavy chain CDR2 and -3 sequences which are at least 50%, 55%, 60% 70%, 80%, 90%, or 95% identical to the heavy chain CDR2 and -3 sequences and light chain CDR1 and -3 sequences which are at least 50%, 55%, 60% 70%, 80%, 90%, or 95% identical to the light chain CDR1 and -3 sequences of the antibodies M14-G07. In a further preferred embodiment the antibodies or antigen-binding fragments thereof comprise heavy chain CDR2 and -3 sequences which are at least 50%, 55%, 60% 70%, 80%, 90%, or 95% identical to the heavy chain CDR2 and -3 sequences and light chain CDR1 and -3 sequences which are at least 50%, 55%, 60% 70%, 80%, 90%, or 95% identical to the light chain CDR1 and -3 sequences of the antibodies M18-G08, M18-G08-G or M18-G08-G-DKTHT.

In a further preferred embodiment the antibodies or antigen-binding fragments thereof of the invention comprise a variable heavy chain sequence which is at least 50%, 60%, 70%, 80%, 90%, 92% or 95% identical to a VH sequence disclosed in table 1 or table 3, preferably of the antibodies M14-G07, M18-G08, M18-G08-G or M18-G08-G-DKTHT. In a further preferred embodiment the antibodies of the invention or antigen-binding fragments thereof comprise a variable light chain sequence which is at least 50%, 60%, 70%, 80%, 90%, 92% or 95% identical to a VL sequence disclosed in table 1 or table 2, preferably of the antibodies M14-G07, M18-G08, M18-G08-G or M18-G08-G-DKTHT.

In a further preferred embodiment the antibodies of the invention or antigen-binding fragments thereof comprise variable heavy and light chain sequences that are at least 50%, 60%, 70%, 80%, 90%, 92% or 95% identical to the VH and VL sequence of the antibodies M14-G07, M18-G08, M18-G08-G or M18-G08-G-DKTHT, respectively.

In a further preferred embodiment the antibodies of the invention or antigen-binding fragments thereof comprise heavy and light chain CDR sequences which conform to the M14-G07 or M18-G08 derived, preferably corresponding, CDR consensus sequences as depicted in table 4 and 5. A further preferred embodiment are antibodies of the invention or antigen-binding fragments thereof comprising heavy chain CDR sequences conforming to the corresponding heavy chain CDR sequences as represented by the consensus sequences SEQ ID NO: 497 (CDR H1), SEQ ID NO: 222 (CDR H2) and SEQ ID NO: 498 (CDR H3), and light chain CDR sequences conforming to the corresponding light chain CDR sequences as represented by the consensus sequences SEQ ID NO: 499 (CDR L1), SEQ ID NO: 500 (CDR L2) and SEQ ID NO: 501 (CDR L3), or comprising heavy chain CDR sequences conforming to the corresponding heavy chain CDR sequences as represented by the consensus sequences SEQ ID NO: 502 (CDR H1), SEQ ID NO: 503 (CDR H2) and SEQ ID NO: 504 (CDR H3), and light chain CDR sequences conforming to the corresponding light chain CDR sequences as represented by the consensus sequences SEQ ID NO: 505 (CDR L1), SEQ ID NO: 506 (CDR L2) and SEQ ID NO: 507 (CDR L3).

In a further preferred embodiment the antibodies of the invention or antigen-binding antibody fragments comprise at least one, preferably corresponding, heavy and/or light chain CDR sequence as disclosed in table 1 or table 2 and 3, or preferably of an antibody as depicted in table 1 or table 2 and 3. In a further preferred embodiment the antibodies or antigen-binding antibody fragments comprise at least one, two, three, four, five or six, preferably corresponding, heavy and light chain CDR sequences as disclosed in table 1 or table 2 and 3, or preferably of an antibody as depicted in table 1 or table 2 and 3. In a further preferred embodiment the antibodies or antigen-binding antibody fragments comprise the heavy or light chain CDR1, CDR2 or CDR3 sequences of an antibody as depicted in table 1 or table 2 and 3, the heavy or light chain CDR1 and CDR2 sequences of an antibody as depicted in table 1 or table 2 and 3, the heavy or light chain CDR1 and CDR3 sequences of an antibody as depicted in table 1 or table 2 and 3, the heavy or light chain CDR2 and CDR3 sequences of an antibody as depicted in table 1 or table 2 and 3, the heavy or light chain CDR1, CDR2 and CDR3 sequences of an antibody as depicted in table for table 2 and 3. In a further preferred embodiment the antibodies or antigen-binding antibody fragments comprise the heavy chain CDR sequences CDR1 and CDR2 and the light chain CDR sequences CDR1, CDR2, CDR3 of an antibody as depicted in table 1 or table 2 and 3. In a further preferred embodiment the antibodies or antigen-binding antibody fragments comprise the heavy and light chain CDR1, CDR2 or CDR3 sequences of an antibody as depicted in table 1 or table 2 and 3, the heavy and light chain CDR1 and CDR2 sequences of an antibody as depicted in table or table 2 and 3, the heavy and light chain CDR1 and CDR3 sequences of an antibody as depicted in table 1 or table 2 and 3, the heavy and light chain CDR2 and CDR3 sequences of an antibody as depicted in table 1 or table 2 and 3, the heavy and light chain CDR1, CDR2 and CDR3 sequences of an antibody as depicted in table 1 or table 2 and 3.

In a further preferred embodiment the antibodies or antigen-binding antibody fragments of the invention comprise the heavy and light chain CDR sequences of an antibody as depicted in table 1 or table 2 and 3.

In a further embodiment the antibodies or antigen-binding antibody fragments of the invention comprise a VH and/or VL sequence disclosed in table 1 or table 2 and 3. In a further preferred embodiment the antibodies or antigen-binding antibody fragments comprise the VH and VL sequence of an antibody depicted in table 1 or table 2 and 3.

In a further embodiment the antibodies or antigen-binding antibody fragments of the invention comprise a VH and/or VL sequence disclosed in table 9 (variants of M14-G07) or table 11 (variants of M18-G08) depecting single and/or double amino acid substitutions introduced into the heavy and/or light chain of said molecules according to column 2.

In a preferred embodiment the antibodies or antigen-binding antibody fragments of the invention are monoclonal. In a further preferred embodiment the antibodies or antigen-binding antibody fragments of the invention are human, humanized or chimeric.

Throughout this document, reference is made to the following preferred antibodies of the invention: “M14-G07”, “M18-G08”, “M18-G08-G” or “M18-G08-DKTHT” or “M18-G08-G-DKTHT”

M14-G07 represents an antibody comprising a variable heavy chain region corresponding to SEQ ID NO: 475 (DNA)/SEQ ID NO: 207 (protein) and a variable light chain region corresponding to SEQ ID NO: 476 (DNA)/SEQ ID NO: 208 (protein).

M18-G08 represents an antibody comprising a variable heavy chain region corresponding to SEQ ID NO: 485 (DNA)/SEQ ID NO: 217 (protein) and a variable light chain region corresponding to SEQ ID NO: 486 (DNA)/SEQ ID NO: 218 (protein).

M18-G08-G represents an antibody comprising a variable heavy chain region corresponding to SEQ ID NO: 385 (DNA)/SEQ ID NO: 117 (protein) and a variable light chain region corresponding to SEQ ID NO: 386 (DNA)/SEQ ID NO: 118 (protein).

M18-G08-G-DKTHT represents an antibody comprising a heavy chain region corresponding to SEQ ID NO: 491 (DNA)/SEQ ID NO: 489 (protein) and a light chain region corresponding to SEQ ID NO: 492 (DNA)/SEQ ID NO: 490 (protein).

M18-G08-DKTHT represents an antibody comprising a heavy chain region corresponding to SEQ ID NO: 495 (DNA)/SEQ ID NO: 493 (protein) and a light chain region corresponding to SEQ ID NO: 496 (DNA)/SEQ ID NO: 494 (protein).

M018-G08-G-IgG1 represents an IgG1 antibody comprising a heavy chain region corresponding to SEQ ID NO: 508 (protein) and a light chain region corresponding to SEQ ID NO: 509 (protein).

In some embodiments, the antibody, antigen-binding fragment thereof, or derivative thereof or antibody mimetic or nucleic acid encoding the same is isolated. An isolated biological component (such as a nucleic acid molecule or protein such as an antibody) is one that has been substantially separated or purified away from other biological components in the cell of the organism in which the component naturally occurs, e.g., other chromosomal and extra-chromosomal DNA and RNA, proteins and organelles. Nucleic acids and proteins that have been “isolated” include nucleic acids and proteins purified by standard purification methods Sambrook et al., 1989 (Sambrook, J., Fritsch, E. F. and Maniatis, T. (1989) Molecular Cloning: A laboratory manual, Cold Spring Harbor Laboratory Press, Cold Spring Harbor, USA) and Robert K. Scopes eat al 1994 Protein Purification, —Principles and Practice, Springer Science and Business Media LLC. The term also embraces nucleic acids and proteins prepared by recombinant expression in a host cell as well as chemically synthesized nucleic acids.

### Antibody Generation

A fully human n-CoDeR antibody phage display library was used to isolate high affinity, human monoclonal antibodies and antigen-binding fragments thereof specific for FXa inhibitors comprising structure formula 1 using specifically developed tools and methods. These tools and methods include specific target molecules and their immoblization to surfaces based on the biotin-streptavidin interaction. Immobilization of FXa inhibitors comprising structure formula 1 as target molecules is a prerequisite for the selection of antibodies and antigen binding fragments thereof from phage libraries (phage panning) and for screening and analyses of specific antibodies in the ELISA-format.

Inventive antibodies and antigen-binding fragments thereof were developed by a combination of three non-conventional approaches in phage-display technology (PDT). First, FXa inhibitors comprising structure formula 1 which can be immobilized to surfaces based on the biotin-streptavidin interaction were synthesized (Example 1K and 1L). Second, target compounds (Example 1K and 1L) immobilized on streptavidin beads were used for selections under stringent conditions. Pre-adsorption of the phage library with FITC-biotin was included to deplete binder specific for the biotin-linker part. Third, screening methods were developed which allowed for successive screening of the phage outputs obtained in the various panning rounds. The combination of these specific methods allowed the isolation of the unique antibodies “M16-D05”, “M14-G07”, “M15-B07”, “M25-E05”, “M18-A10”, “M16-A03” and “M18-G08”.

These unique antibodies were further characterized in terms of binding affinity to target molecules in ELISA-tests and SPR-analysis (BIAcore) and in functional neutralization assays using e.g. rivaroxaban as FXa inhibitor.

Variants of the unique antibodies “M14-G07” and “M18-G08” were generated and screened for affinity and/or functionality in reversing the effect of rivaroxaban in FXa assays. The resulting variant “M18-G08-G” was recloned and expressed as the non-tagged Fab “M18-G08-G-DKTHT” and in-depth characterized. as described in some of the examples.

Other Exemplary Methods for Obtaining Inventive Antibodies and Functional Antibody Fragments Thereof or Antibody Mimetics:

In a similar manner as described above a skilled person can generate antibody mimetics by library screening.

In addition to the use of display libraries, other methods can be used to obtain inventive antibodies or functional fragments thereof. For example, compounds from Example 1K and/or Example 1L coupled to carrier proteins can be used as an antigen in a non-human animal, e.g., a rodent. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies (Mabs) derived from the genes with the desired specificity may be produced and selected. See, e.g., XENOMOUSE™, Green et al., 1994, Nat. Gen. 7: 13-21; U.S. 2003-0070185, WO 96134096, published Oct. 31, 1996, and PCT Application No. PCT1US96105928, filed Apr. 29, 1996.

In another embodiment, a monoclonal antibody is obtained from the non-human animal, and then modified, e.g., humanized or deimmunized. Winter describes a CDR-grafting method that may be used to prepare the humanized antibodies (UK Patent Application GB 2 188638A, filed on Mar. 26, 1987; U.S. Pat. No. 5,225,539). All of the CDRs of a particular human antibody may be replaced with at least a portion of a non-human CDR or only some of the CDRs may be replaced with non-human CDRs. It is only necessary to replace the number of CDRs required for binding of the humanized antibody to a predetermined antigen. Humanized antibodies can be generated by replacing sequences of the Fv variable region that are not directly involved in antigen binding with equivalent sequences from human Fv variable regions. General methods for generating humanized antibodies are provided by Morrison, S. L., 1985, Science 229:1202-1207, by Oi et al., 1986, 25 BioTechniques 4:214, and by Queen et al. U.S. Pat. Nos. 5,585,089, U.S. Pat. No. 5,693,761 and U.S. Pat. No. 5,693,762. Those methods include isolating, manipulating, and expressing the nucleic acid sequences that encode all or part of immunoglobulin Fv variable regions from at least one of a heavy or light chain. Numerous sources of such nucleic acid are available. For example, nucleic acids may be obtained from a hybridoma producing an antibody against a predetermined target, as described above. The recombinant DNA encoding the humanized antibody, or fragment thereof, can then be cloned into an appropriate expression vector.

### Peptide Variants

Antibodies or antigen-binding fragments of the invention are not limited to the specific peptide sequences provided herein. Rather, the invention also embodies variants of these polypeptides. With reference to the instant disclosure and conventionally available technologies and references, the skilled worker will be able to prepare, test and utilize functional variants of the antibodies and antigen-binding fragments thereof disclosed herein, while appreciating that variants having the ability to bind to anticoagulants fall within the scope of the present invention.

A variant can include, for example, an antibody or antigen-binding fragment thereof that has at least one altered complementary determining region (CDR) (hyper-variable) and/or framework (FR) (variable) domain/position, vis-á-vis a peptide sequence disclosed herein. To better illustrate this concept, a brief description of antibody structure follows.

q´chain) or three (heavy chain) constant domains and a variable region (VL, VH), the latter of which is in each case made up of four FR regions and three interspaced CDRs. The antigen-binding site is formed by one or more CDRs, yet the FR regions provide the structural framework for the CDRs and, hence, play an important role in antigen binding. By altering one or more amino acid residues in a CDR or FR region, the skilled worker routinely can generate mutated or diversified antibody sequences, which can be screened against the antigen, for new or improved properties, for example.

Tables 2 (VL) and 3 (VH) delineate the CDR and FR regions for certain antibodies of the invention and compare amino acids at a given position to each other and to corresponding consensus sequences.

TABLE 2
VL Sequences

| | | |
|---|---|---|
| M14-G07 | (1) | QSVLTQPPSASGTPGQRVTISCSGSSSNIGSNYVYWYQQLPGTAPKLLIYDNNDRPSGV |
| M14-G07-10 | (1) | QSVLTQPPSASGTPGQRVTISCSGSSRNIGSFYVYWYQQLPGTAPKLLIYDNNQRPSGV |
| M14-G07-35 | (1) | QSVLTQPPSASGTPGQRVTISCSGSSSNIGSNYVYWYQQLPGTAPKLLIYDNNQRPSGV |
| M14-G07-36 | (1) | QSVLTQPPSASGTPGQRVTISCSGSSSNIGSYYVYWYQQLPGTAPKLLIYDNNQRPSGV |
| M14-G07-37 | (1) | QSVLTQPPSASGTPGQRVTISCSGSSSNIGSYYVYWYQQLPGTAPKLLIYDNNQRPSGV |
| M18-G08 | (1) | QSVLTQPPSASGTPGQRVTISCSGSSSDIGSNTVNWYQQLPGTAPKLLIYDNNQRPSGV |
| M18-G08-2 | (1) | QSVLTQPPSASGTPGQRVTISCSGSSSNIGSNKVNWYQQLPGTAPKVLIWSNNQRPSGV |
| M18-G08-10 | (1) | QSVLTQPPSASGTPGQRVTISCSGSSSNIGSNKVNWYQQLPGTAPKLLIWSNNQRPSGV |
| M18-G08-18 | (1) | QSVLTQPPSASGTPGQRVTISCSGSSSNIGSNKVNWYQQLPGTAPKVLIYSNNQRPSGV |
| M18-G08-34 | (1) | QSVLTQPPSASGTPGQRVTISCSGSSSNIGSNKVNWYQQLPGTAPKSLIWSNNQRPSGV |
| M18-G08-41 | (1) | QSVLTQPPSASGTPGQRVTISCSGSSSNIGSNTVNWYQQLPGTAPKLLIWSNNQRPSGV |
| M18-G08-G | (1) | QSVLTQPPSASGTPGQRVTISCSGSSSNIGSNTVNWYQQLPGTAPKLLIYSNNQRPSGV |
| | | ----LCDR1---- -LCDR2- |
| M14-G07 | (60) | PDRFSGSKSGTSASLAISGLRSEDEADYYCVAWDDSLNGHWVFGGGTKLTVL |
| M14-G07-10 | (60) | PDRFSGSKSGTSASLAISGLRSEDEADYYCVAWDDSWSGHWVFGGGTKLTVL |
| M14-G07-35 | (60) | PDRFSGSKSGTSASLAISGLRSEDEADYYCVAWDDSLSGHWVFGGGTKLTVL |
| M14-G07-36 | (60) | PDRFSGSKSGTSASLAISGLRSEDEADYYCVAWDDSLSGHWVFGGGTKLTVL |
| M14-G07-37 | (60) | PDRFSGSKSGTSASLAISGLRSEDEADYYCVAWDDSWSGHWVFGGGTKLTVL |
| M18-G08 | (60) | PDRFSGSKSGTSASLAISGLRSEDEADYYCQSYDSSLSG-WVFGGGTKLTVL |
| M18-G08-2 | (60) | PDRFSGSKSGTSASLAITGLQAEDEADYYCQSYDSSLVG-WVFGGGTKLTVL |
| M18-G08-10 | (60) | PDRFSGSKSGTSASLAITGLQAEDEADYYCQSYDSSLVG-WVFGGGTKLTVL |
| M18-G08-18 | (60) | PDRFSGSKSGTSASLAITGLQAEDEADYYCQSYDSSLVG-WVFGGGTKLTVL |
| M18-G08-34 | (60) | PDRFSGSKSGTSASLAITGLQAEDEADYYCQSYDSSLVG-WVFGGGTKLTVL |
| M18-G08-41 | (60) | PDRFSGSKSGTSASLAITGLQAEDEADYYCQSYDSSLSG-WVFGGGTKLTVL |
| M18-G08-G | (60) | PDRFSGSKSGTSASLAITGLQAEDEADYYCQSYDSSLSG-WVFGGGTKLTVL |
| | | ----LCDR3--- |

TABLE 3
VH Sequences

| | | |
|---|---|---|
| M14-G07 | (1) | EVQLLESGGGLVQPGGSLRLSCAASGFTFGDYAMSWVRQAPGKGLEWVSGISGSGGSTY |
| M14-G07-10 | (1) | EVQLLESGGGLVQPGGSLRLSCAASGFTFSSYAMSWVRQAPGKGLEWVGGISGSGGSTY |
| M14-G07-35 | (1) | EVQLLESGGGLVQPGGSLRLSCAASGFTFSSYAMSWVRQAPGKGLEWVSGISGSGGSTY |
| M14-G07-36 | (1) | EVQLLESGGGLVQPGGSLRLSCAASGFTFSSYAMSWVRQAPGKGLEWVSGISGSGGSTY |
| M14-G07-37 | (1) | EVQLLESGGGLVQPGGSLRLSCAASGFTFSSYAMSWVRQAPGKGLEWVSGISGSGGSTY |
| M18-G08 | (1) | EVQLLESGGGLVQPGGSLRLSCAASGFTFSNAWMSWVRQAPGKGLEWVSSISSSSGYIY |
| M18-G08-2 | (1) | EVQLLESGGGLVQPGGSLRLSCAASGFTFSDYWMSWVRQAPGKGLEWVSSISTSSSYIY |
| M18-G08-10 | (1) | EVQLLESGGGLVQPGGSLRLSCAASGFTFSDSWMSWVRQAPGKGLEWVSSISTSSSYIY |
| M18-G08-18 | (1) | EVQLLESGGGLVQPGGSLRLSCAASGFTFSDYWMSWVRQAPGKGLEWVSSISTSSSYIY |
| M18-G08-34 | (1) | EVQLLESGGGLVQPGGSLRLSCAASGFTFSDYWMSWVRQAPGKGLEWVSSISTSSSYIY |
| M18-G08-41 | (1) | EVQLLESGGGLVQPGGSLRLSCAASGFTFSDYWMSWVRQAPGKGLEWVSSISTSSSYIY |
| M18-G08-G | (1) | EVQLLESGGGLVQPGGSLRLSCAASGFTFSSYWMSWVRQAPGKGLEWVSSISSSSSYIY |
| | | HCDR1 -----HCDR2- |
| M14-G07 | (60) | YADSVKGRFTISRDNSKNTLYLQMNSLRAEDTAVYYCAREGETSFGLDVWGQGTLVTVTS |
| M14-G07-10 | (60) | YADSVKGRFTISRDNSKNTLYLQMNSLRAEDTAVYYCARQGRTSFYLDVWGQGTLVTVSS |
| M14-G07-35 | (60) | YADSVKGRFTISRDNSKNTLYLQMNSLRAEDTAVYYCAREGETSFYLDVWGQGTLVTVSS |
| M14-G07-36 | (60) | YADSVKGRFTISRDNSKNTLYLQMNSLRAEDTAVYYCAREGETSFYLDVWGQGTLVTVSS |
| M14-G07-37 | (60) | YADSVKGRFTISRDNSKNTLYLQMNSLRAEDTAVYYCAREGETSFYLDVWGQGTLVTVSS |
| M18-G08 | (60) | YADSLKGRFTISRDNSKNTLYLQMNSLRAEDTAVYYCARVWRNH--LDYWGQGTLVTVTS |
| M18-G08-2 | (60) | YADSVKGRFTISRDNSKNTLYLQMNSLRAEDTAVYYCARVWRNY--LDYWGQGTLVTVSS |
| M18-G02-10 | (60) | YADSVKGRFTISRDNSKNTLYLQMNSLRAEDTAVYYCARVWRNS--LDYWGQGTLVTVSS |
| M18-G08-18 | (60) | YADSVKGRFTISRDNSKNTLYLQMNSLRAEDTAVYYCARVWRNY--LSYWGQGTLVTVSS |
| M18-G08-34 | (60) | YADSVKGRFTISRDNSKNTLYLQMNSLRAEDTAVYYCARVWRNY--LSYWGQGTLVTVSS |
| M18-G08-41 | (60) | YADSVKGRFTISRDNSKNTLYLQMNSLRAEDTAVYYCARVWRNA--LSYWGQGTLVTVSS |
| M18-G08-G | (60) | YADSVKGRFTISRDNSKNTLYLQMNSLRAEDTAVYYCARVWRNY--LDYWGQGTLVTVSS |
| | | ------- --HCDR3--- |

TABLE 4
Consensus CDR sequences of M14-G07 derivatives

| CDR H1 M14-G07 SEQ ID NO: 497 | | | | | | | | | | | | | | | | | |
|---|---|---|---|---|---|---|---|---|---|---|---|---|---|---|---|---|---|
| position | 1 | | 2 | 3 | 4 | 5 | | | | | | | | | | | |
| amino acid | D | | Y | A | M | S | | | | | | | | | | | |
| variant | S | | | | | | | | | | | | | | | | |
| consensus | D or S | | Y | A | M | S | | | | | | | | | | | |
| CDR H2 M14-G07 SEQ ID NO: 222 | | | | | | | | | | | | | | | | | |
| position | 1 | 2 | 3 | 4 | 5 | 6 | 7 | 8 | 9 | 10 | 11 | 12 | 13 | 14 | 15 | 16 | 17 |
| amino acid | G | I | S | G | S | G | G | S | T | Y | Y | A | D | S | V | K | G |
| variant | | | | | | | | | | | | | | | | | |
| consensus | G | I | S | G | S | G | G | S | T | Y | Y | A | D | S | V | K | G |

CDR H3 M14-G07 SEQ ID NO: 496

| position | 1 | 2 | 3 | 4 | 5 | 6 | 7 | 8 | 9 | 10 |
|---|---|---|---|---|---|---|---|---|---|---|
| amino acid | E | G | E | T | S | F | G | L | D | V |
| variant | Q | | R or K | | | | Y | | | |
| consensus | Q or E | G | R, K or | T | S | F | Y or G | L | D | V |
| | | | E | | | | | | | |

| CDR L1 M14-G07 SEQ ID NO: 499 | | | | | | | | | | | | | |
|---|---|---|---|---|---|---|---|---|---|---|---|---|---|
| position | 1 | 2 | 3 | 4 | 5 | 6 | 7 | 8 | 9 | 10 | 11 | 12 | 13 |
| amino acid | S | G | S | S | S | N | I | G | S | N | Y | V | Y |
| variant | C | | | T | | | | | A | F or Y | | | |
| consensus | C or S | G | S | S or T | S or R | N | I | G | S or A | F, Y or | Y | V | Y |
| | | | | | | | | | | N | | | |

| CDR L2 M14-G07 SEQ ID NO: 500 | | | | | | | |
|---|---|---|---|---|---|---|---|
| position | 1 | 2 | 3 | 4 | 5 | 6 | 7 |
| amino acid | D | N | N | D | R | P | S |
| variant | | | R | Q | | | |
| consensus | D | N | N or R | Q or D | R | P | S |

CDR L3 M14-G07 SEQ ID NO: 501

| position | 1 | 2 | 3 | 4 | 5 | 6 | 7 | 8 | 9 | 10 | 11 | 12 |
|---|---|---|---|---|---|---|---|---|---|---|---|---|
| amino acid | V | A | W | D | D | S | L | N | G | H | W | V |
| variant | | | | | W or V | | W or Y | S | | T | | |
| consensus | V | A | W | D | D, V or | S | W, Y or | S or N | G | H or T | W | V |
| | | | | | W | | L | | | | | |

TABLE 5
Consensus CDR sequences of M18-G08 derivatives

| CDR H1 M18-G08 SEQ ID NO: 502 | | | | | |
|---|---|---|---|---|---|
| position | 1 | 2 | 3 | 4 | 5 |
| amino acid | N | A | W | M | S |
| variant | S or D | Y, S or | | | |
| | | H | | | |
| consensus | D, N or | A, Y, S | W | M | S |
| | S | or H | | | |

| CDR H2 M18-G08 SEQ ID NO: 503 | | | | | | | | | | | | | | | | | |
|---|---|---|---|---|---|---|---|---|---|---|---|---|---|---|---|---|---|
| position | 1 | 2 | 3 | 4 | 5 | 6 | 7 | 8 | 9 | 10 | 11 | 12 | 13 | 14 | 15 | 16 | 17 |
| amino acid | S | I | S | S | S | S | G | Y | I | Y | Y | A | D | S | L | K | G |
| variant | | | | T | | | S | | | | | | | G | V | | |
| consensus | S | I | S | S or T | S | S | G or S | Y | I | Y | Y | A | D | G or S | L or V | K | G |

CDR H3 M18-G08 SEQ ID NO: 504

| position | 1 | 2 | 3 | 4 | 5 | 6 | 7 | 8 |
|---|---|---|---|---|---|---|---|---|
| amino acid | V | W | R | N | H | L | D | Y |
| variant | | | | | Y, S, or | | D | |
| | | | | | A | | | |
| consensus | V | W | R | N | Y, S, A | L | D or S | Y |
| | | | | | or H | | | |

CDR L1 M18-G08 SEQ ID NO: 505

| position | 1 | 2 | 3 | 4 | 5 | 6 | 7 | 8 | 9 | 10 | 11 | 12 | 13 |
|---|---|---|---|---|---|---|---|---|---|---|---|---|---|
| amino acid | S | G | S | S | S | D | I | G | S | N | T | V | N |
| variant | | | | | | N | | | | | T | | |
| consensus | S | G | S | S | S | N or D | I | G | S | N | K or T | V | N |

CDR L2 M18-G08 SEQ ID NO: 506

| position | 1 | 2 | 3 | 4 | 5 | 6 | 7 |
|---|---|---|---|---|---|---|---|
| amino acid | D | N | N | Q | R | P | S |
| variant | S | | | | H | | N |
| consensus | D or S | N | N | Q | R or H | P | S or N |

| CDR L3 M18-G08 SEQ ID NO: 507 | | | | | | | | | | | |
|---|---|---|---|---|---|---|---|---|---|---|---|
| position | 1 | 2 | 3 | 4 | 5 | 6 | 7 | 8 | 9 | 10 | 11 |
| amino acid | Q | S | Y | D | S | S | L | S | G | W | V |
| variant | | | | S | | | | V | | | |
| consensus | Q | S | Y | D or S | S | S | L | S or V | G | W | V |

A further preferred embodiment of the invention is an antibody or antigen binding fragment thereof in which the CDR sequences are selected as shown in table 1.

A further preferred embodiment of the invention is an antibody or antigen-binding fragment in which the VH and VL sequences are selected as shown in table 1. The skilled worker can use the data in tables 1, 2 and 3 to design peptide variants that are within the scope of the present invention. It is preferred that variants are constructed by changing amino acids within one or more CDR regions; a variant might also have one or more altered framework regions. Alterations also may be made in the framework regions. For example, a peptide FR domain might be altered where there is a deviation in a residue compared to a germline sequence.

Furthermore, variants may be obtained by using one antibody as starting point for optimization by diversifying one or more amino acid residues in the antibody, preferably amino acid residues in one or more CDRs, and by screening the resulting collection of antibody variants for variants with improved properties. Particularly preferred is diversification of one or more amino acid residues in CDR3 of VL and/or VH. Diversification can be done by synthesizing a collection of DNA molecules using trinucleotide mutagenesis (TRIM) technology (Virnekäs B. et al., Nucl. Acids Res. 1994, 22: 5600). Antibodies or antigen-binding fragments thereof include molecules with modifications/variations including but not limited to e.g. modifications leading to altered half-life (e.g. modification of the Fc part or attachment of further molecules such as PEG).

### Conservative Amino Acid Variants

Polypeptide variants may be made that conserve the overall molecular structure of an antibody peptide sequence described herein. Given the properties of the individual amino acids, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

For example, (a) nonpolar (hydrophobic) amino acids include alanine, leucine, isoleucine, valine, proline, phenylalanine, tryptophane, and methionine; (b) polar neutral amino acids include glycine, serine, threonine, cysteine, tyrosine, asparagine, and glutamine; (c) positively charged (basic) amino acids include arginine, lysine, and histidine; and (d) negatively charged (acidic) amino acids include aspartic acid and glutamic acid. Substitutions typically may be made within groups (a)-(d). In addition, glycine and proline may be substituted for one another based on their ability to disrupt α-helices. Similarly, certain amino acids, such as alanine, cysteine, leucine, methionine, glutamic acid, glutamine, histidine and lysine are more commonly found in α-helices, while valine, isoleucine, phenylalanine, tyrosine, tryptophan and threonine are more commonly found in β-pleated sheets. Glycine, serine, aspartic acid, asparagine, and proline are commonly found in turns. Some preferred substitutions may be made among the following groups: (i) S and T; (ii) P and G; and (iii) A, V, L and I. Given the known genetic code, and recombinant and synthetic DNA techniques, the skilled scientist readily can construct DNAs encoding the conservative amino acid variants.

As used herein, “sequence identity” between two polypeptide sequences, indicates the percentage of amino acids that are identical between the sequences. “Sequence homology” indicates the percentage of amino acids that either is identical or that represent conservative amino acid substitutions.

### DNA Molecules of the Invention

The present invention also relates to the DNA molecules that encode an antibody of the invention or antigen-binding fragment thereof. These sequences include, but are not limited to, those DNA molecules set forth in table 1.

DNA molecules of the invention are not limited to the sequences disclosed herein, but also include variants thereof. DNA variants within the invention may be described by reference to their physical properties in hybridization. The skilled worker will recognize that DNA can be used to identify its complement and, since DNA is double stranded, its equivalent or homolog, using nucleic acid hybridization techniques. It also will be recognized that hybridization can occur with less than 100% complementarity. However, given appropriate choice of conditions, hybridization techniques can be used to differentiate among DNA sequences based on their structural relatedness to a particular probe. For guidance regarding such conditions see, Sambrook et al., 1989 supra and Ausubel et al., 1995 (Ausubel, F. M., Brent, R., Kingston, R. E., Moore, D. D., Sedman, J. G., Smith, J. A., & Struhl, K. eds. (1995). Current Protocols in Molecular Biology. New York: John Wiley and Sons).

Structural similarity between two polynucleotide sequences can be expressed as a function of “stringency” of the conditions under which the two sequences will hybridize with one another. As used herein, the term “stringency” refers to the extent that the conditions disfavor hybridization. Stringent conditions strongly disfavor hybridization, and only the most structurally related molecules will hybridize to one another under such conditions. Conversely, non-stringent conditions favor hybridization of molecules displaying a lesser degree of structural relatedness. Hybridization stringency, therefore, directly correlates with the structural relationships of two nucleic acid sequences. The following relationships are useful in correlating hybridization and relatedness (where Tₘ is the melting temperature of a nucleic acid duplex):

Tₘ=69.3+0.41(G+C)% a.

The Tₘ of a duplex DNA decreases by 1° C. with every increase of 1% in the number of mismatched base pairs. b.

(Tₘ)_{μ2}−(Tₘ)_{μ1}=18.5 log₁₀μ2/μ1 c.

where n1 and n2 are the ionic strengths of two solutions.

Hybridization stringency is a function of many factors, including overall DNA concentration, ionic strength, temperature, probe size and the presence of agents which disrupt hydrogen bonding. Factors promoting hybridization include high DNA concentrations, high ionic strengths, low temperatures, longer probe size and the absence of agents that disrupt hydrogen bonding. Hybridization typically is performed in two phases: the “binding” phase and the “washing” phase.

First, in the binding phase, the probe is bound to the target under conditions favoring hybridization. Stringency is usually controlled at this stage by altering the temperature. For high stringency, the temperature is usually between 65° C. and 70° C., unless short (<20 nt) oligonucleotide probes are used. A representative hybridization solution comprises 6×SSC, 0.5% SDS, 5×Denhardt's solution and 100 ng of nonspecific carrier DNA. See Ausubel et al., section 2.9, supplement 27 (1994). Of course, many different, yet functionally equivalent, buffer conditions are known. Where the degree of relatedness is lower, a lower temperature may be chosen. Low stringency binding temperatures are between about 25° C. and 40° C. Medium stringency is between at least about 40° C. to less than about 65° C. High stringency is at least about 65° C.

Second, the excess probe is removed by washing. It is at this phase that more stringent conditions usually are applied. Hence, it is this “washing” stage that is most important in determining relatedness via hybridization. Washing solutions typically contain lower salt concentrations. One exemplary medium stringency solution contains 2×SSC and 0.1% SDS. A high stringency wash solution contains the equivalent (in ionic strength) of less than about 0.2×SSC, with a preferred stringent solution containing about 0.1×SSC. The temperatures associated with various stringencies are the same as discussed above for “binding.” The washing solution also typically is replaced a number of times during washing. For example, typical high stringency washing conditions comprise washing twice for 30 minutes at 55° C. and three times for 15 minutes at 60° C.

An embodiment of the invention is an isolated nucleic acid sequence that encodes (i) the antibody or antigen-binding fragment of the invention, the CDR sequences as depicted in table 1, or (ii) the variable light and heavy chain sequences as depicted in table 1, or (iii) which comprises a nucleic acid sequence that encodes an antibody or antigen-binding fragment of the invention, the CDR sequences as depicted in table 1, or the variable light and heavy chain sequences as depicted in table 1.

### Functionally Equivalent Variants

Yet another class of DNA variants within the scope of the invention may be described with reference to the product they encode. These functionally equivalent polynucleotides are characterized by the fact that they encode the same peptide sequences found in table 1, due to the degeneracy of the genetic code.

It is recognized that variants of DNA molecules provided herein can be constructed in several different ways. For example, they may be constructed as completely synthetic DNAs. Methods of efficiently synthesizing oligonucleotides in the range of 20 to about 150 nucleotides are widely available. See Ausubel et al., section 2.11, Supplement 21 (1993). Overlapping oligonucleotides may be synthesized and assembled in a fashion first reported by Khorana et al., J. Mol. Biol. 72:209-217 (1971); see also Ausubel et al., supra, Section 8.2. Synthetic DNAs preferably are designed with convenient restriction sites engineered at the 5′ and 3′ ends of the gene to facilitate cloning into an appropriate vector.

As indicated, a method of generating variants is to start with one of the DNAs disclosed herein and then to conduct site-directed mutagenesis. See Ausubel et al., supra, chapter 8, Supplement 37 (1997). In a typical method, a target DNA is cloned into a single-stranded DNA bacteriophage vehicle. Single-stranded DNA is isolated and hybridized with an oligonucleotide containing the desired nucleotide alteration(s). The complementary strand is synthesized and the double stranded phage is introduced into a host. Some of the resulting progeny will contain the desired mutant, which can be confirmed using DNA sequencing. In addition, various methods are available that increase the probability that the progeny phage will be the desired mutant. These methods are well known to those in the field and kits are commercially available for generating such mutants.

### Recombinant DNA Constructs and Expression

The present invention further provides recombinant DNA constructs comprising one or more of the nucleotide sequences of the present invention. The recombinant constructs of the present invention are used in connection with a vector, such as a plasmid, phagemid, phage or viral vector, into which a DNA molecule encoding an antibody of the invention or antigen-binding fragment thereof is inserted.

An antibody, antigen binding portion, or derivative thereof provided herein can be prepared by recombinant expression of nucleic acid sequences encoding light and heavy chains or portions thereof in a host cell. To express an antibody, antigen binding portion, or derivative thereof recombinantly, a host cell can be transfected with one or more recombinant expression vectors carrying DNA fragments encoding the light and/or heavy chains or portions thereof such that the light and heavy chains are expressed in the host cell. Standard recombinant DNA methodologies are used prepare and/or obtain nucleic acids encoding the heavy and light chains, incorporate these nucleic acids into recombinant expression vectors and introduce the vectors into host cells, such as those described in Sambrook, Fritsch and Maniatis (eds.), Molecular Cloning; A Laboratory Manual, Second Edition, Cold Spring Harbor, N.Y., (1989), Ausubel, F. M. et al. (eds.) Current Protocols in Molecular Biology, Greene Publishing Associates, (1989) and in U.S. Pat. No. 4,816,397 by Boss et al.

In addition, the nucleic acid sequences encoding variable regions of the heavy and/or light chains can be converted, for example, to nucleic acid sequences encoding full-length antibody chains, Fab fragments, or to scFv. The VL- or VH-encoding DNA fragment can be operatively linked, (such that the amino acid sequences encoded by the two DNA fragments are in-frame) to another DNA fragment encoding, for example, an antibody constant region or a flexible linker. The sequences of human heavy chain and light chain constant regions are known in the art (see e.g., Kabat, E. A., et al. (1991) Sequences of Proteins of Immunological Interest, Fifth Edition, U.S. Department of Health and Human Services, NIH Publication No. 91-3242) and DNA fragments encompassing these regions can be obtained by standard PCR amplification.

To create a polynucleotide sequence that encodes a scFv, the VH- and VL-encoding nucleic acids can be operatively linked to another fragment encoding a flexible linker such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., Nature (1990) 348:552-554).

To express the antibodies, antigen binding portions or derivatives thereof standard recombinant DNA expression methods can be used (see, for example, Goeddel; Gene Expression Technology. Methods in Enzymology 185, Academic Press, San Diego, Calif. (1990)). For example, DNA encoding the desired polypeptide can be inserted into an expression vector which is then transfected into a suitable host cell. Suitable host cells are prokaryotic and eukaryotic cells. Examples for prokaryotic host cells are e.g. bacteria, examples for eukaryotic host cells are yeast, insect or mammalian cells. In some embodiments, the DNAs encoding the heavy and light chains are inserted into separate vectors. In other embodiments, the DNA encoding the heavy and light chains are inserted into the same vector. It is understood that the design of the expression vector, including the selection of regulatory sequences is affected by factors such as the choice of the host cell, the level of expression of protein desired and whether expression is constitutive or inducible.

### Bacterial Expression

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and, if desirable, to provide amplification within the host. Suitable prokaryotic hosts for transformation include E. coli, Bacillus subtilis, Salmonella typhimurium and various species within the genera Pseudomonas, Streptomyces, and Staphylococcus.

Bacterial vectors may be, for example, bacteriophage-, plasmid- or phagemid-based. These vectors can contain a selectable marker and bacterial origin of replication derived from commercially available plasmids typically containing elements of the well known cloning vector pBR322 (ATCC 37017). Following transformation of a suitable host strain and growth of the host strain to an appropriate cell density, the selected promoter is de-repressed/induced by appropriate means (e.g., temperature shift or chemical induction) and cells are cultured for an additional period. Cells are typically harvested by centrifugation, disrupted by physical or chemical means, and the resulting crude extract retained for further purification.

In bacterial systems, a number of expression vectors may be advantageously selected depending upon the use intended for the protein being expressed. For example, when a large quantity of such a protein is to be produced, for the generation of antibodies or to screen peptide libraries, for example, vectors which direct the expression of high levels of fusion protein products that are readily purified may be desirable. Antibodies of the present invention or antigen-binding fragment thereof or antibody mimetics include naturally purified products, products of chemical synthetic procedures, and products produced by recombinant techniques from a prokaryotic host, including, for example, E. coli, Bacillus subtilis, Salmonella typhimurium and various species within the genera Pseudomonas, Streptomyces, and Staphylococcus, preferably, from E. coli cells.

### Mammalian Expression & Purification

Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al. The recombinant expression vectors can also include origins of replication and selectable markers (see e.g., U.S. Pat. Nos. 4,399,216, 4,634,665 and U.S. Pat. No. 5,179,017, by Axel et al.). Suitable selectable markers include genes that confer resistance to drugs such as G418, hygromycin or methotrexate, on a host cell into which the vector has been introduced. For example, the dihydrofolate reductase (DHFR) gene confers resistance to methotrexate and the neo gene confers resistance to G418.

Transfection of the expression vector into a host cell can be carried out using standard techniques such as electroporation, calcium-phosphate precipitation, and DEAE-dextran, lipofection or polycation-mediated transfection.

Suitable mammalian host cells for expressing the antibodies, antigen binding fragements, or derivatives thereof, or antibody mimetics provided herein include Chinese Hamster Ovary (CHO cells) (including dhfr-CHO cells, described in Urlaub and ChasM, (1980) Proc. Natl. Acad. Sci. USA 77:4216-4220, used with a DHFR selectable marker, e.g., as described in R. J. Kaufman and P. A. Sharp (1982) Mol. Biol. 159:601-621, NSO myeloma cells, COS cells and SP2 cells. In some embodiments, the expression vector is designed such that the expressed protein is secreted into the culture medium in which the host cells are grown. Transient transfection/epression of antibodies can for example be achieved following the protocols by Durocher et al (2002) Nucl. Acids Res. Vol 30 e9. Stable transfection/expression of antibodies can for example be achieved following the protocols of the UCOE system (T. Benton et al. (2002) Cytotechnology 38: 43-46).

The antibodies, antigen binding fragments, or derivatives thereof can be recovered from the culture medium using standard protein purification methods.

Antibodies of the invention or antigen-binding fragments thereof or antibody mimetics can be recovered and purified from recombinant cell cultures by well-known methods including, but not limited to ammonium sulfate or ethanol precipitation, acid extraction, Protein A chromatography, Protein G chromatography, anion or cation exchange chromatography, phospho-cellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Antibodies of the present invention or antigen-binding fragments thereof include naturally purified products, products of chemical synthetic procedures, and products produced by recombinant techniques from a eukaryotic host, including, for example, yeast (for example Pichia), higher plant, insect and mammalian cells, preferably from mammalian cells. Depending upon the host employed in a recombinant production procedure, the antibody of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20.

### Therapeutic Methods

Therapeutic methods involve administering to a subject in need of treatment a therapeutically effective amount of an inventive antibody or antigen-binding fragment or antibody mimetic. A “therapeutically effective” amount hereby is defined as the amount of an inventive antibody or antigen-binding fragment or antibody mimetic that is of sufficient quantity to neutralize FXa inhibitor comprising the structure of formula 1 in plasma, either as a single dose or according to a multiple dose regimen, alone or in combination with other agents, which leads to the alleviation of an adverse condition, yet which amount is toxicologically tolerable. An inventive antibody or antigen-binding fragment thereof or antibody mimetic might be co-administered with known medicaments, and in some instances the antibody or antigen-binding fragment thereof or antibody mimetic might itself be modified. For example, an antibody or antigen-binding fragment thereof or antibody mimetic could be conjugated or added to polyethylene glycol, carrier proteins, liposomes and encapsulating agents, phospholipid membranes or nanoparticles to increase plasma half life of an antidote.

The present invention relates to a therapeutic method of selectively neutralizing the anticoagulant effect of a FXa inhibitor comprising the structure of formula 1 in a subject undergoing anticoagulant therapy with said FXa inhibitors by administering to the subject an effective amount of antibody or antigen-binding fragment thereof or antibody mimetic. It is contemplated that the antibody or antigen-binding fragment of the invention or antibody mimetic can be used in elective or emergency situations to safely and specifically neutralize anticoagulant properties of said FXa inhibitors resulting in approximately normalized coagulation status. Such elective or emergency situations are situations were a normalized coagulation is favorable, including severe bleeding events (e.g. caused by trauma) or a need for an urgent invasive procedure (e.g. an emergency surgery). The antibody or antigen-binding fragment of the invention does not have an instrinsic effect on hemodynamic parameters. In a preferred embodiment the FXa inhibitor is rivaroxaban.

The subject may be a human or non-human animal (e.g., rabbit, rat, mouse, dog, monkey or other lower-order primate).

In one embodiment, the antibody or antigen-binding fragment of the invention or antibody mimetic is administered after the administration of an overdose of a FXa inhibitor comprising the structure of formula 1.

In another embodiment the antibody or antigen-binding fragment of the invention or antibody mimetic is administered prior to a surgery, which may expose subjects treated with a FXa inhibitor comprising the structure of formula 1 to an increased bleeding risk.

In still another embodiment, a subject treated with an antibody or antigen-binding fragment of the invention or antibody mimetic in order to neutralize the effect of a FXa inhibitor comprising the structure of formula 1 on coagulation can be rapidly re-anticoagulated by administering a FXa-inhibitor which is not bound by the antidote.

It is contemplated that an effective amount of the antibody or antigen-binding fragment of the invention or antibody mimetic is administered to the subject.

In another embodiment, the antibody or antigen-binding fragment of the invention or antibody mimetic is administered in combination with a coagulant agent, having anti-thrombotic and/or anti-fibrinolytic activity. In one embodiment, the blood coagulation agent is selected from the group consisting of a coagulation factor, a polypeptide related to the coagulation factor, a recombinant coagulation factor and combinations thereof. In another embodiment, the blood coagulating agent may be selected from the group consisting of an adsorbent chemical, a hemostatic agent, thrombin, fibrin glue, desmopressin, cryoprecipitate and fresh frozen plasma, coagulation factor concentrate, activated or non-activated prothrombin complex concentrate, FEIBA, platelet concentrates and combinations thereof. More examples of available blood coagulation factors are available in the citation Brooker M, Registry of Clotting Factor Concentrates, 8th Edition, World Federation of Hemophilia, 2008.

The disorders mentioned above have been well characterized in humans, but also exist with a similar etiology in other animals, including mammals, and can be treated by administering pharmaceutical compositions of the present invention.

To treat any of the foregoing disorders, pharmaceutical compositions for use in accordance with the present invention may be formulated in a conventional manner using one or more physiologically acceptable carriers or excipients. An antibody and antigen-binding fragment of the invention can be administered by any suitable means, which can vary, depending on the type of disorder being treated. Possible administration routes include parenteral (e.g., intramuscular, intravenous, intra-arterial, intraperitoneal, or subcutaneous), intrapulmonary and intranasal, and, if desired for local immunosuppressive treatment, intralesional administration. In addition, an antibody of the invention or antigen-binding fragment thereof might be administered by pulse infusion, with, e.g., declining doses of the antibody or antigen binding fragment. Preferably, the dosing is given by injections, most preferably intravenous or subcutaneous injections, depending in part on whether the administration is brief or chronic. The amount to be administered will depend on a variety of factors such as the clinical symptoms, weight of the individual, whether other drugs are administered. The skilled artisan will recognize that the route of administration will vary depending on the disorder or condition to be treated.

Determining a therapeutically effective amount of the antibody or antigen-binding fragment thereof or antibody mimetic, according to this invention, largely will depend on particular patient characteristics, route of administration, and the nature of the disorder being treated. General guidance can be found, for example, in the publications of the International Conference on Harmonization and in REMINGTON'S PHARMACEUTICAL SCIENCES, chapters 27 and 28, pp. 484-528 (18th ed., Alfonso R. Gennaro, Ed., Easton, Pa.: Mack Pub. Co., 1990). More specifically, determining a therapeutically effective amount will depend on such factors as toxicity and efficacy of the medicament. Toxicity may be determined using methods well known in the art and found in the foregoing references. Efficacy may be determined utilizing the same guidance in conjunction with the methods described below in the Examples.

### Diagnostic Methods

An other aspect of the invention is an in vitro diagnostic method to determine whether an altered coagulation status of a subject is due to the presence of a FXa inhibitor comprising the structure of formula 1 in the blood of said subject, wherein (a) an in vitro coagulation test is performed in the presence of an inventive antibody or antigen-binding fragment, (b) an in vitro coagulation test is performed in the absence of an inventive antibody or antigen-binding fragment, (c) the results of the test performed in step (a) and (b) are compared, and (d) an altered coagulation status due to the presence of a FXa inhibitor comprising the structure of formula 1 is diagnosed, if results from steps (a) and (b) are different. A preferred in vitro coagulation test is a PT, aPTT or thrombin generation test. The rapid availability of this information can be very important for planning further steps in diagnostic and therapy, especially in emergency situations. Prolonged clotting time in laboratory testing (e.g. PTT) can be observed for example in the presence of lupus anticoagulants, where autoantibodies against phospholipids and proteins associated with cell membranes are interfering with the normal coagulation process. However, in vivo lupus anticoagulant is actually a prothrombotic agent, as it precipitates the formation of thrombi by interacting with platelet membrane phospholipids and increasing adhesion and aggregation of platelets. In combination with other assays, the diagnostic test described above may help to detect lupus anticoagulants.

An other aspect of the invention is an in vitro diagnostic method to determine the amount of functional active inventive antibody or antigen-binding fragment thereof or antibody mimetic in the blood of a subject treated with said molecules using compounds from Example 1K and/or 1L as a capturing reagent. E.g. in an ELISA-assay, compounds from Example 1K and/or 1L can be immobilized to streptavidin-coated wells and samples containing inventive antibody or antigen-binding fragment thereof or antibody mimetic can be added. Subsequent to a washing step, captured said molecules can be detected with a detection antibody and the amount of material in the sample can be calculated by comparing results to a calibration curve with known amounts of antibody or antigen-binding fragment thereof or antibody mimetic.

An other aspect of the invention is an in vitro diagnostic method to determine the amount of a FXa inhibitor comprising the structure of formula 1 in boodyfluids of a subject treated with said inhibitor using compounds from Example 1K and/or 1L and an inventive antibody or antigen-binding fragment thereof or antibody mimetic as a capturing reagent for an ELISA-test. The amount of bound FXa inhibitor comprising the structure of formula 1 can be estimated from the signal that can be generated by the addition of a labeled anti-ideotypic antibody, whose binding to the inventive antibody or antigen-binding fragment thereof or antibody mimetic is blocked in the presence of said inhibitor

An other aspect of the invention is an in vitro diagnostic method to determine the amount of a FXa inhibitor comprising the structure of formula 1 in boodyfluids of a subject treated with said inhibitor using compounds from Example 1K and/or 1L and an inventive antibody or antigen-binding fragment thereof or antibody mimetic in a competition binding assay. In more detail, bodyfluids, e.g. plasma from a subject treated with said inhibitor, can be preincubated with a fixe amount of the inventive antibody or antigen-binding fragment thereof or antibody mimetic. Subsequently, residual binding of the inventive antibody to immobilized compounds from Example 1K and/or 1L, can be assessed e.g. in an ELISA-assay. The amount of said inhibitor in the sample can be calculated by comparing results to a calibration curve with known amounts of inhibitor.

In a preferred embodiment bodyfluids are for example urine, blood, blood plasma, blood serum and saliva. In another preferred embodiment the bodyfluid is blood.

Another embodiment of the invention is a diagnostic kit comprising an anticoagulant tethered to a matrix and an antibody or antigen-binding fragment thereof of the invention, binding to said anticoagulant. The tethering can be by a linker, e.g. a biotin linker. The matrix can be a solid matrix, e.g. a microtiter plate. In a preferred embodiment of the above diagnostic kit the anticoagulant is rivaroxaban. In a further preferred embodiment of the above diagnostic kit the tethered anticoagulant is compound Example 1K or compound Example 1L. In a further preferred embodiment of the above diagnostic kit the antibody is M18-G08, M18-G08-G, or M18-G08-G-DKTHT or antigen-binding fragment thereof. A most preferred kit comprises antibody M18-G08-G-DKTHT or antigen-binding fragment thereof and compound Example 1K. In a further embodiment the aforementioned diagnostic kit is used in a diagnostic method to quantitatively and/or qualitatively determine an anticoagulant (wherein the anticoagulant corresponds to the anticoagulant of the kit) in a sample comprising the steps (a) forming a mixture of an antibody or antigen-binding fragment thereof of the aforementioned kit under conditions allowing binding of the antibody to the anticoagulant, (b) contacting of said mixture with the tethered anticoagulant of the aforementioned kit under conditions allowing binding of the antibody to the anticoagulant, (c) determine the amount of antibody or antigen-binding fragment bound to the tethered anticoagulant. The amount of said anticoagluant in the sample can be calculated by comparing the results to a calibration curve with known amounts of said anticoagulant. In a preferred embodiment the sample is a bodyfluid. More preferred are bodyfluids comprised in a group of fluids consisting of urine, blood, blood plasma, blood serum and saliva. In a preferred embodiment the above diagnostic method is for the determination of rivaroxaban. Preferably, the method employs a kit comprising antibody M18-G08-G-DKTHT or antigen-binding fragment thereof.and compound Example 1K. An example for such a diagnostic method is the is a competing ELISA format method depicted in Example 22.

### Pharmaceutical Compositions and Administration

The present invention also relates to pharmaceutical compositions which may comprise inventive antibodies and antigen-binding fragments, alone or in combination with at least one other agent, such as stabilizing compound, which may be administered in any sterile, biocompatible pharmaceutical carrier, including, but not limited to, saline, buffered saline, dextrose, and water. Any of these molecules can be administered to a patient alone, or in combination with other agents, drugs or hormones, in pharmaceutical compositions where it is mixed with excipient(s) or pharmaceutically acceptable carriers. In one embodiment of the present invention, the pharmaceutically acceptable carrier is pharmaceutically inert.

The present invention also relates to the administration of pharmaceutical compositions. Such administration is accomplished orally or parenterally. Methods of parenteral delivery include topical, intra-arterial, intramuscular, subcutaneous, intramedullary, intrathecal, intraventricular, intravenous, intraperitoneal, or intranasal administration. In addition to the active ingredients, these pharmaceutical compositions may contain suitable pharmaceutically acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Ed. Maack Publishing Co, Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient.

Pharmaceutical preparations for oral use can be obtained through combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl, cellulose, hydroxypropylmethylcellulose, or sodium carboxymethyl cellulose; and gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinyl pyrrolidone, carbopol gel, polyethylene glycol and/or titanium dioxide, lacquer solutions, and suitable organic solvents or solvent mixtures. Dyestuffs or pigments may be added to the tablets or dragee coatings for product identification or to characterize the quantity of active compound, i.e. dosage.

Pharmaceutical preparations that can be used orally include push-fit capsules made of gelatin, as well as soft, sealed capsules made of gelatin and a coating such as glycerol or sorbitol. Push-fit capsules can contain active ingredients mixed with a filler or binders such as lactose or starches, lubricants such as talc or magnesium stearate, and optionally, stabilizers. In soft capsules, the active compounds may be dissolved or suspended in suitable liquids, such as fatty oils, liquid paraffin, or liquid polyethylene glycol with or without stabilizers.

Pharmaceutical formulations for parenteral administration include aqueous solutions of active compounds. For injection, the pharmaceutical compositions of the invention may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

For topical or nasal administration, penetrants appropriate to the particular barrier to be permeated are used in the formulation. Such penetrants are generally known in the art.

### Kits

The invention further relates to pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

In another embodiment, the kits may contain DNA sequences encoding the antibodies or antigen-binding fragments of the invention. Preferably the DNA sequences encoding these antibodies are provided in a plasmid suitable for transfection into and expression by a host cell. The plasmid may contain a promoter (often an inducible promoter) to regulate expression of the DNA in the host cell. The plasmid may also contain appropriate restriction sites to facilitate the insertion of other DNA sequences into the plasmid to produce various antibodies. The plasmids may also contain numerous other elements to facilitate cloning and expression of the encoded proteins. Such elements are well known to those of skill in the art and include, for example, selectable markers, initiation codons, termination codons, and the like.

### Manufacture and Storage

The pharmaceutical compositions of the present invention may be manufactured in a manner that is known in the art, e.g., by means of conventional mixing, dissolving, granulating, dragee-making, levigating, emulsifying, encapsulating, entrapping or lyophilizing processes.

The pharmaceutical composition may be provided as a salt and can be formed with acids, including by not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

After pharmaceutical compositions comprising a compound of the invention formulated in an acceptable carrier have been prepared, they can be placed in an appropriate container and labeled for treatment of an indicated condition. For administration of inventive antibodies and antigen-binding fragments, such labeling would include amount, frequency and method of administration.

### Therapeutically Effective Dose.

Pharmaceutical compositions suitable for use in the present invention include compositions wherein the active ingredients are contained in an effective amount to achieve the intended purpose, i.e. neutralization of a FXa inhibitor comprising the structure of formula 1. The determination of an effective dose is well within the capability of those skilled in the art.

For any compound, the therapeutically effective dose can be estimated initially either in in vitro coagulation tests, e.g., PT, or in animal models, usually mice, rabbits, dogs, or pigs. The animal model is also used to achieve a desirable concentration range and route of administration. Such information can then be used to determine useful doses and routes for administration in humans.

A therapeutically effective dose refers to that amount of antibodies or antigen-binding fragments thereof or antibody mimetic that ameliorate the symptoms or condition. Therapeutic efficacy and toxicity of such compounds can be determined by standard pharmaceutical procedures in vitro or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred. The data obtained from in vitro assays and animal studies are used in formulating a range of dosage for human use. The dosage of such compounds lies preferably within a range of circulating concentrations what include the ED₅₀ with little or no toxicity. The dosage varies within this range depending upon the dosage form employed, sensitivity of the patient, and the route of administration.

The antibody or antigen-binding fragment of this invention or antibody mimetic may be administered once or several times when needed to neutralize the effect of a FXa inhibitor comprising the structure of formula 1 present in a subject's plasma. Preferably, the antibody or antigen-binding fragment of this invention are sufficient when administering in a single dose.

The exact dosage is chosen by the individual physician in view of the patient to be treated. Dosage and administration are adjusted to provide sufficient levels of the active moiety or to maintain the desired effect. Additional factors that may be taken into account include the identity and/or amount of FXa inhibitor comprising the structure of formula 1, which was administered to the subject, the formulation and/or the mode of administration of the antibody or antigen-binding fragment thereof; age, weight and gender of the patient; diet, time and frequency of administration, drug combination(s), reaction sensitivities, and tolerance/response to therapy.

Normal dosage amounts may vary from 0.1 to 100,000 milligrams total dose, depending upon the route of administration. Guidance as to particular dosages and methods of delivery is provided in the literature. See U.S. Pat. Nos. 4,657,760; 5,206,344; or 5,225,212. Those skilled in the art will employ different formulations for polynucleotides than for proteins or their inhibitors. Similarly, delivery of polynucleotides or polypeptides will be specific to particular cells, conditions, locations, etc. Preferred specific activities for a radiolabelled antibody may range from 0.1 to 10 mCi/mg of protein (Riva et al., Clin. Cancer Res. 5:3275-3280, 1999; Ulaner et al., 2008 Radiology 246(3):895-902)

The present invention is further described by the following examples. The examples are provided solely to illustrate the invention by reference to specific embodiments. These exemplifications, while illustrating certain specific aspects of the invention, do not portray the limitations or circumscribe the scope of the disclosed invention.

All examples were carried out using standard techniques, which are well known and routine to those of skill in the art, except where otherwise described in detail. Routine molecular biology techniques of the following examples can be carried out as described in standard laboratory manuals, such as Sambrook et al., Molecular Cloning: A Laboratory Manual, 2nd Ed.; Cold Spring Harbor Laboratory Press, Cold Spring Harbor, N.Y., 1989.

### EXAMPLES

### A) Examples

### Abbreviations

aq. aqueous solution
cat. Catalytic
d day(s)
DCI direct chemical ionization (in MS)
DMAP 4-dimethylaminopyridine
DMF Dimethylformamide
DMSO dimethyl sulphoxide
EDC N′-(3-dimethylaminopropyl)-N-ethylcarbodiimide x HCl
EI electron impact ionization (in MS)
ESI electrospray ionization (in MS)
Et Ethyl
GC-MS gas chromatography-coupled mass spectroscopy
h hour(s)
HATU O-(7-azabenzotriazol-1-yl)-N,N,N′,N′-tetramethyluronium hexafluorophosphate
HOBt 1H-1,2,3-benzotriazole-1-ol hydrate
HPLC high pressure, high performance liquid chromatography
conc. Concentrated
LC-MS liquid chromatography-coupled mass spectroscopy
Meth. Method
min minute(s)
MS mass spectroscopy
NMR nuclear magnetic resonance spectroscopy
Rₜ retention time (in HPLC)
RT room temperature
TFA trifluoroacetic acid
THF Tetrahydrofuran

LC-MS Methods:

Method 1A:

instrument: Micromass QuattroPremier with Waters UPLC Acquity; column: Thermo Hypersil GOLD 1.9μ 50 mm×1 mm; mobile phase A: 1 l of water+0.5 ml of 50% strength formic acid, mobile phase B: 1 l acetonitrile+0.5 ml of 50% strength formic acid; gradient: 0.0 min 90% A→0.1 min 90% A→1.5 min 10% A→2.2 min 10% A; flow rate: 0.33 ml/min; oven: 50° C.; UV detection: 210 nm

Method 2A:

instrument: Micromass Quattro Micro MS with HPLC Agilent series 1100; column: Thermo Hypersil GOLD 3μ 20 mm×4 mm; mobile phase A: 1 l of water+0.5 ml of 50% strength formic acid, mobile phase B: 1 l acetonitrile+0.5 ml of 50% strength formic acid; gradient: 0.0 min 100% A→3.0 min 10% A→4.0 min 10% A 4.01 min 100% A (flow rate 2.5 ml/min)→5.00 min 100% A; oven: 50° C.; flow rate: 2 ml/min; UV detection: 210 nm

Method 3A:

Instrument: Waters ACQUITY SQD UPLC System; column: Waters Acquity UPLC HSS T3 1.8μ 50 mm×1 mm; mobile phase A: 1 l of water+0.25 ml of 99% strength formic acid, mobile phase B: 1 l of acetonitrile+0.25 ml of 99% strength formic acid; gradient: 0.0 min 90% A→1.2 min 5% A→2.0 min 5% A; oven: 50° C.; flow rate: 0.40 ml/min; UV detection: 210-400 nm.

Method 4A:

Instrument: Waters ZQ with HPLC Agilent Serie 1100; UV DAD; column: Thermo Hypersil GOLD 3μ 20 mm×4 mm; mobile phase A: 1 l of water+0.5 ml of 50% strength formic acid, mobile phase B: 1 l of acetonitrile+0.5 ml of 50% strength formic acid; gradient: 0.0 min 100% A→3.0 min 10% A→4.0 min 10% A; oven: 55° C.; flow rate: 2 ml/min; UV detection: 210 nm

Preparative Separation of Enantiomers:

Method 1B:

Phase: spherical vinyl silica gel bound methacryl-L-leucine-tert.-butylamide, 670 mm×40 mm; mobile phase: ethyl acetate; flow rate: 80 ml/min, UV detection: 265 nm

Method 2B:

Phase: spherical vinyl silica gel bound methacryl-L-leucine-dicyclopropylmethylamide, 670 mm×40 mm; mobile phase A: ethyl acetate, mobile phase B: methanol; gradient: 0.0 min 100% A→10.1 min 100% A→13.1 min 100% B→13.11 min 100% A→21.0 min 100% A; flow rate: 80 ml/min, UV detection: 265 nm

Analytic Separation of Enantiomers:

Method 1C:

Phase: spherical vinyl silica gel bound methacryl-L-leucine-dicyclopropylmethylamide, 250 mm×4.6 mm; mobile phase: ethyl acetate; flow rate: 2 ml/min, UV detection: 265 nm.

### Starting Materials

### Example 1A

### N-({(5S)-3-[4-(2-Allyl-3-oxomorpholin-4-yl)phenyl]-2-oxo-1,3-oxazolidin-5-yl}methyl)-5-chlorothiophene-2-carboxamide [mixture of diastereomers]

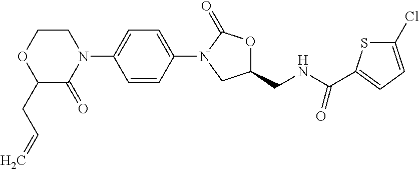 [SMILES: C=CCC1OCCN(c2ccc(N3C[C@H](CNC(=O)c4ccc(Cl)s4)OC3=O)cc2)C1=O]

10.9 g (25.0 mmol) of 5-chloro-N-({(5S)-2-oxo-3-[4-(3-oxo-4-morpholinyl)phenyl]-1,3-oxazolidin-5-yl}methyl)-2-thiophenecarboxamide (described in WO 01/047919) were dissolved in 250 ml THF and 62.5 ml (10.5 g, 62.5 mmol) of a 1 N lithium hexamethyldisilazide-THF-solution were added slowly at −78° C. After 30 minutes 2.4 ml (4.4 g, 26.2 mmol) 3-iodo-2-propene were added dropwise. The reaction mixture was allowed to warm slowly to room temperature and was stirred at this temperature for 16 h. Then saturated aqueous ammonium chloride solution and ethyl acetate were added. The phases were separated and the aqueous phase was extracted with ethyl acetate. The combined organic extracts were washed with water, dried over sodium sulphate, filtered and concentrated under reduced pressure. The residue was dissolved in dichlormethane and was purified by column chromatography on silica gel (mobile phase: gradient ethyl acetate/dichloromethane 2:1->ethyl acetate). Yield: 5.8 g (49% of theory)

LC-MS (method 3A): Rt==0.97 min; MS (ESIpos): m/z=476 [M+H]+.

### Example 1B

### N-({(5S)-3-[4-(2-Allyl-3-oxomorpholin-4-yl)phenyl]-2-oxo-1,3-oxazolidin-5-yl}methyl)-5-chlorothiophene-2-carboxamide [enantiomerically pure diastereomer 2]

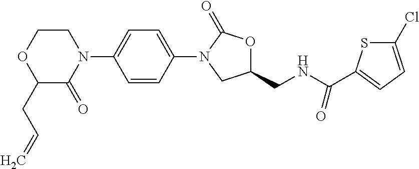 [SMILES: C=CCC1OCCN(c2ccc(N3C[C@H](CNC(=O)c4ccc(Cl)s4)OC3=O)cc2)C1=O]

Separation of isomers of 5.7 g (12.0 mmol) of the compound from Example 1A following method 1B resulted in 2.5 g of Example 1B (second eluated compound).

LC-MS (method 3A): Rt==0.95 min; MS (ESIpos): m/z=476 [M+H]+.

HPLC (method 1C): Rt==4.15 min

### Example 1C

### 5-Chloro-N-{[(5S)-3-{4-[2-(3-hydroxypropyl)-3-oxomorpholin-4-yl]phenyl}-2-oxo-1,3-oxazolidin-5-yl]methyl}thiophene-2-carboxamide [enantiomeric ally pure diastereomer]

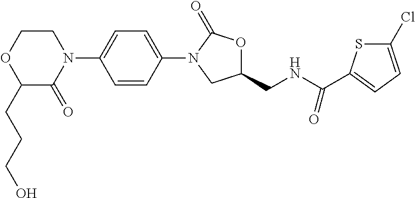 [SMILES: O=C(NC[C@H]1CN(c2ccc(N3CCOC(CCCO)C3=O)cc2)C(=O)O1)c1ccc(Cl)s1]

2.5 g (5.25 mmol) of the compound from Example 1B were dissolved in 35 ml THF and 23.1 ml (1.41 g, 11.6 mmol) of a 0.5 molar THF-solution of 9-borabicyclo[3.3.1]nonane were added at 10 to 15° C. The reaction mixture was allowed to warm to room temperature and was stirred at this temperature for 1.5 h. 13.1 ml (1.05 g, 26.3 mmol) of a 2N sodium hydroxide solution were added dropwise at 0 to 5° C. Then 4.6 ml of a 36% solution of hydrogen peroxide were added dropwise, whereas the bath temperature does not rise above 30° C. After 30 minutes ethyl acetate and water were added. The organic phase was separated. The aqueous phase was extracted with ethyl acetate. The combined organic extracts were washed with aqueous sodium hydrogen sulfite solution, dried over sodium sulphate, filtered and concentrated under reduced pressure. The residue was mixed at 30 to 35° C. with ethyl acetate, filtered and washed with ethyl acetate. The residue was dried under reduced pressure and the crude product was reacted further without further purification.

LC-MS (method 3A): Rt=0.82 min; MS (ESIpos): m/z=494 [M+H]+.

### Example 1D

### 4-[3-(4-{4-[(5S)-5-({[(5-Chloro-2-thienyl)carbonyl]amino}methyl)-2-oxo-1,3-oxazolidin-3-yl]-phenyl}-3-oxomorpholin-2-yl)propoxy]-4-oxobutanoic acid [enantiomerically pure diastereomer]

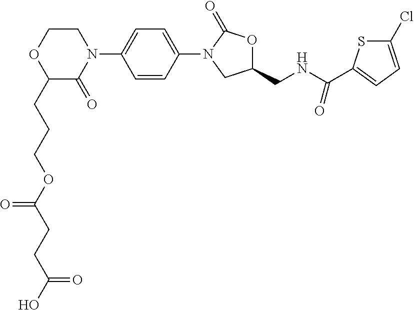 [SMILES: O=C(O)CCC(=O)OCCCC1OCCN(c2ccc(N3C[C@H](CNC(=O)c4ccc(Cl)s4)OC3=O)cc2)C1=O]

To 300 mg (0.61 mmol) of the compound from Example 1C, 182 mg (1.82 mmol) succinic anhydride, 0.23 ml (226 mg, 2.85 mmol) pyridine and 223 mg (1.82 mmol) DMAP were added and were solved in 2 ml DMF. The reaction mixture was stirred at room temperature for 1 h. The reaction mixture was purified by preparative HPLC. Yield: 134 mg (37% of theory)

LC-MS (method 1A): Rt=0.98 min; MS (ESIpos): m/z=594 [M+H]+.

### Example 1E

### tert.-Butyl-(5-{[6-({5-[(3aS,4S,6aR)-2-oxohexahydro-1H-thieno[3,4-d]imidazol-4-yl]pentanoyl}-amino)hexanoyl]amino}pentyl)carbamate

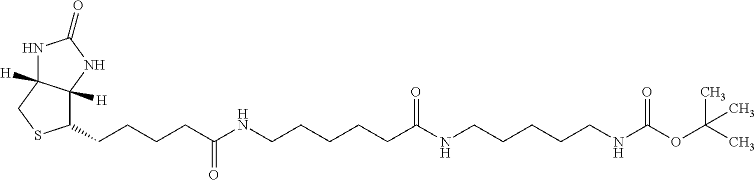

A suspension of 500 mg (1.40 mmol) N-(+)-biotinyl-6-aminocapronic acid, 283 mg (1.40 mmol) tert.-butyl-(5-aminopentyl)carbamate, 321 mg (2.10 mmol) HOBT, 0.24 ml (181 mg, 1.40 mmol) N,N-diisopropylethylamine and 322 mg (1.68 mmol) EDC in 30 ml DMF was stirred at room temperature for 16 h. Then further 291 mg (1.44 ml) tert.-butyl-(5-aminopentyl)carbamate were added and the mixture was stirred at room temperature for further 16 h. The reaction mixture was concentrated under reduced pressure, water and ethyl acetate as well as a small amount of dioxane was added. The aqueous phase was extracted with ethyl acetate several times. The combined organic extracts were dried over sodium sulphate, filtered and concentrated under reduced pressure. The crude product was purified by preparative HPLC. Yield: 100 mg (13% of theory)

LC-MS (method 4A): Rt=1.71 min; MS (ESIpos): m/z=542 [M+H]+.

### Example 1F

### N-(5-Aminopentyl)-6-({5-[(3aS,4S,6aR)-2-oxohexahydro-1H-thieno[3,4-d]imidazol-4-yl]-pentanoyl}amino)hexanamide hydrochloride

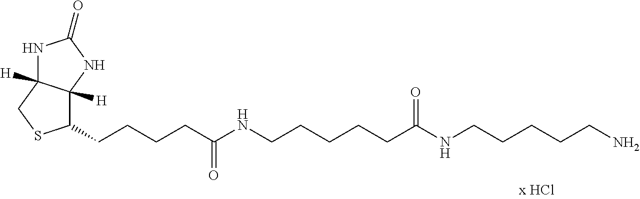

100 mg (0.19 mmol) of the compound from Example 1E were solved in 2 ml methanol and 2 ml dichlormethane and 2 ml (2.0 mmol) of a 1 N hydrochloric acid solution in diethyl ether were added. The mixture was stirred at room temperature for 16 h, concentrated under reduced pressure and dried. Yield: 90 mg (98% of theory)

LC-MS (method 3A): Rt=0.46 min; MS (ESIpos): m/z=442 [M+H−HCl]+.

### Example 1G

### N-({(5S)-3-[4-(2-Allyl-3-oxomorpholin-4-yl)-2-fluorophenyl]-2-oxo-1,3-oxazolidin-5-yl}methyl)-5-chlorothiophene-2-carboxamide [mixture of diastereomers]

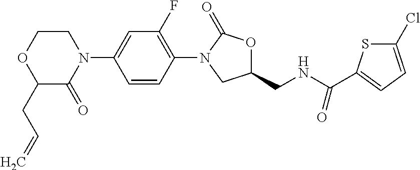 [SMILES: C=CCC1OCCN(c2ccc(N3C[C@H](CNC(=O)c4ccc(Cl)s4)OC3=O)c(F)c2)C1=O]

10.0 g (22.0 mmol) of 5-chloro-N-({(5S)-3-[2-fluoro-4-(3-oxomorpholin-4-yl)phenyl]-2-oxo-1,3-oxazolidin-5-yl}methyl)thiophene-2-carboxamide (described in WO 2008/155034) were dissolved in 220 ml THF and 26.4 ml (4.42 g, 26.4 mmol) of a 1 N lithium hexamethyldisilazide-THF-solution were added slowly at −78° C. After 30 minutes 2.12 ml (3.89 g, 23.1 mmol) 3-iodo-2-propene were added dropwise. The reaction mixture was allowed to warm slowly to room temperature and was stirred at this temperature for 16 h. Then saturated aqueous ammonium chloride solution and ethyl acetate were added. The phases were separated and the aqueous phase was extracted with ethyl acetate. The combined organic extracts were washed with water, dried over sodium sulphate, filtered and concentrated under reduced pressure. Yield: 3.8 g (35% of theory)

LC-MS (method 3A): Rt=0.96 min; MS (ESIpos): m/z=494 [M+H]+.

### Example 111

### N-({(5S)-3-[4-(2-Allyl-3-oxomorpholin-4-yl)-2-fluorophenyl]-2-oxo-1,3-oxazolidin-5-yl}methyl)-5-chlorothiophene-2-carboxamide [enantiomeric ally pure diastereomer 1]

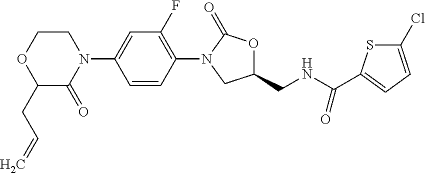 [SMILES: C=CCC1OCCN(c2ccc(N3C[C@H](CNC(=O)c4ccc(Cl)s4)OC3=O)c(F)c2)C1=O]

Separation of isomers of 2.90 g (5.87 mmol) of the compound from Example 1G following method 2B resulted in 1.40 g of Example 1H (first eluated compound).

LC-MS (method 2A): Rt=2.02 min; MS (ESIpos): m/z=494 [M+H]+.

HPLC (method 1C): Rt=2.54 min

### Example 1I

### 5-Chloro-N-{[(5S)-3-{2-fluoro-4-[2-(3-hydroxypropyl)-3-oxomorpholin-4-yl]phenyl}-2-oxo-1,3-oxazolidin-5-yl]methyl}thiophene-2-carboxamide [enantiomerically pure diastereomer]

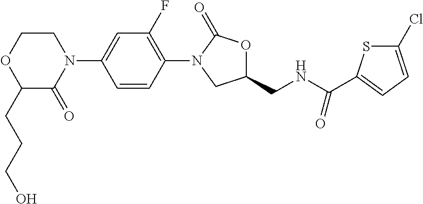 [SMILES: O=C(NC[C@H]1CN(c2ccc(N3CCOC(CCCO)C3=O)cc2F)C(=O)O1)c1ccc(Cl)s1]

1.40 g (2.83 mmol) of the compound from Example 1H were dissolved in 15 ml THF and 12.5 ml (0.76 g, 6.24 mmol) of a 0.5 molaren THF-solution of 9-borabicyclo[3.3.1]nonane were added at 10 to 15° C. The reaction mixture was allowed to warm to room temperature and was stirred at this temperature for 1.5 h. 7.1 ml (0.57 g, 14.2 mmol) of a 2N sodium hydroxide solution were added dropwise at 0 to 5° C. Then 2.5 ml of a 35% solution of hydrogen peroxide were added dropwise, whereas the bath temperature does not rise above 30° C. After 30 minutes ethyl acetate and water were added. The organic phase was separated. The aqueous phase was extracted with ethyl acetate. The combined organic extracts were washed with aqueous sodium hydrogen sulfite solution, dried over sodium sulphate, filtered and concentrated under reduced pressure. The residue was mixed at 30 to 35° C. with ethyl acetate, filtered and washed with ethyl acetate. The residue was dried under reduced pressure and the crude product was reacted further without further purification.

LC-MS (method 3A): Rt=0.83 min; MS (ESIpos): m/z=512 [M+H]+.

### Example 1J

### {4-[(5S)-5-({[(5-Chloro-2-thienyl)carbonyl]amino}methyl)-2-oxo-1,3-oxazolidin-3-yl]-3-fluoro-phenyl}-3-oxomorpholin-2-yl)propoxy]-4-oxobutanoic acid [enantiomerically pure diastereomer]

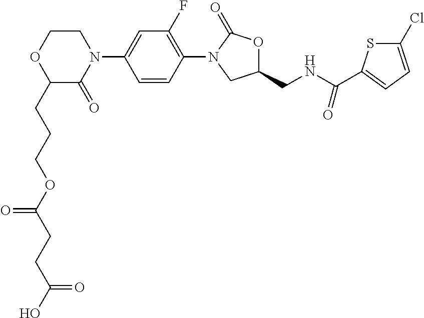 [SMILES: O=C(O)CCC(=O)OCCCC1OCCN(c2ccc(N3C[C@H](CNC(=O)c4ccc(Cl)s4)OC3=O)c(F)c2)C1=O]

To 300 mg (0.59 mmol) of the compound from Example 1I, 176 mg (1.76 mmol) succinic anhydride, 223 μl (218 mg, 1.76 mmol) pyridine and 215 mg (1.76 mmol) N,N-4-dimethylaminopyridine were added and were solved in 1 ml DMF. The reaction mixture was stirred at room temperature for 1 h. The reaction mixture was purified by preparative HPLC. Yield: 132 mg (37% of theory)

LC-MS (method 1A): Rt=0.99 min; MS (ESIpos): m/z=612 [M+H]+.

### Working Examples

### Example 1K

### 3-(4-{4-[(5S)-5-({[(5-Chloro-2-thienyl)carbonyl]amino}methyl)-2-oxo-1,3-oxazolidin-3-yl]-phenyl}-3-oxomorpholin-2-yl)propyl-4-oxo-4-[(5-{[6-({5-[(3aS,4S,6aR)-2-oxohexahydro-1H-thieno[3,4-d]imidazol-4-yl]pentanoyl}amino)hexanoyl]amino}pentyl)amino]butanoate

[enantiomeric ally pure diastereomer]

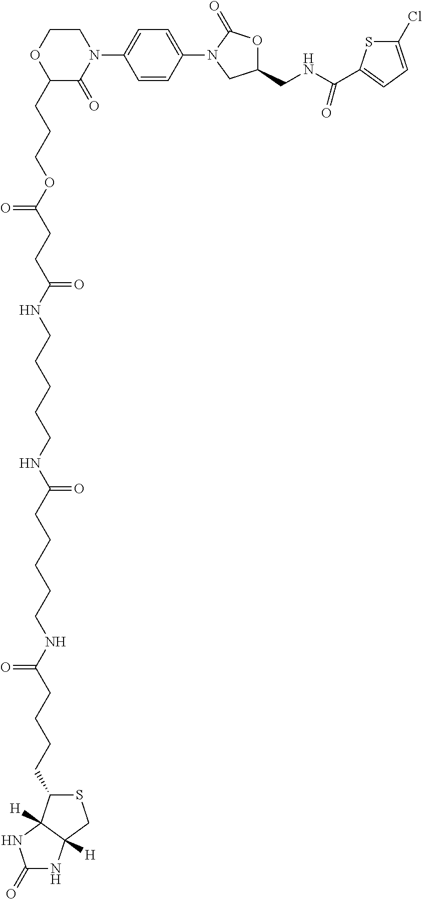 [SMILES: O=C(CCCCCNC(=O)CCCC[C@@H]1SC[C@@H]2NC(=O)N[C@@H]21)NCCCCCNC(=O)CCC(=O)OCCCC1OCCN(c2ccc(N3C[C@H](CNC(=O)c4ccc(Cl)s4)OC3=O)cc2)C1=O]

24 mg (0.05 mmol) of the compound from Example 1F and 30 mg (0.05 mmol) of the compound from Example 1D were solved in 1 ml DMF and then 26 μl (19 mg, 0.15 mmol) N,N-diisopropylethylamine and 29 mg (0.08 mmol) HATU were added. The mixture was stirred at room temperature for 1 h, concentrated under reduced pressure and purified by preparative HPLC. Yield: 9 mg (18% of theory)

LC-MS (method 3A): Rt=0.81 min; MS (ESIpos): m/z=1017 [M+H]+.

¹H-NMR (400 MHz, DMSO-d6) δ=9.05-8.90 (m, 1H), 7.86-7.76 (m, 1H), 7.72-7.66 (m, 3H), 7.55 (d, 2H), 7.37 (d, 2H), 7.19 (d, 1H), 6.47-6.28 (m, 1H), 4.90-4.77 (m, 1H), 4.36-4.26 (m, 1H), 4.25-3.99 (m, 6H), 3.95-3.79 (m, 3H), 3.65-3.52 (m, 3H), 3.14-3.05 (m, 1H), 3.03-2.96 (m, 6H), 2.82 (dd, 1H), 2.39-2.28 (m, 2H), 2.08-1.98 (m, 3H), 1.94-1.86 (m, 1H), 1.82-1.55 (m, 4H), 1.54-1.42 (m, 5H), 1.41-1.17 (m, 15H).

### Example 1L

### 3-(4-{4-[(5S)-5-({[(5-Chloro-2-thienyl)carbonyl]amino}methyl)-2-oxo-1,3-oxazolidin-3-yl]-3-fluorophenyl}-3-oxomorpholin-2-yl)propyl-4-oxo-4-[(5-{[6-({5-[(3aS,4S,6aR)-2-oxohexahydro-1H-thieno[3,4-d]imidazol-4-yl]pentanoyl}amino)hexanoyl]amino}pentyl)amino]butanoate [enantiomerically pure diastereomer]

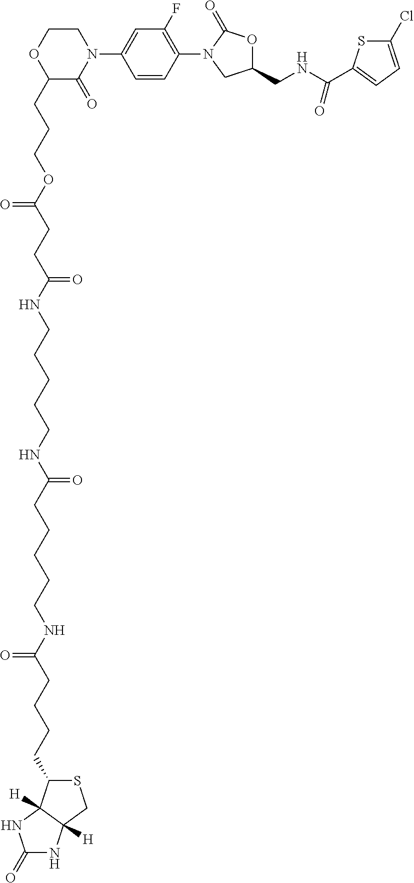 [SMILES: O=C(CCCCCNC(=O)CCCC[C@@H]1SC[C@@H]2NC(=O)N[C@@H]21)NCCCCCNC(=O)CCC(=O)OCCCC1OCCN(c2ccc(N3C[C@H](CNC(=O)c4ccc(Cl)s4)OC3=O)c(F)c2)C1=O]

24 mg (0.05 mmol) of the compound from Example 1F and 31 mg (0.05 mmol) of the compound from Example 1J were solved in 1 ml DMF and then 26 μl (19 mg, 0.15 mmol) N,N-diisopropylethylamine and 29 mg (0.08 mmol) HATU were added. The mixture was stirred at room temperature for 1 h, concentrated under reduced pressure and purified by preparative HPLC. Yield: 40 mg (73% of theory)

LC-MS (method 1A): Rt=0.98 min; MS (ESIpos): m/z=1035 [M+H]+;

¹H-NMR (400 MHz, DMSO-d6) δ=9.00-8.96 (m, 1H), 7.81 (t, 1H), 7.75-7.63 (m, 3H), 7.58-7.40 (m, 2H), 7.28 (dd, 1H), 7.21 (d, 1H), 6.46-6.31 (m, 2H), 4.93-4.82 (m, 1H), 4.36-4.27 (m, 1H), 4.27-4.18 (m, 1H), 4.15-4.05 (m, 3H), 4.05-3.99 (m, 2H), 3.95-3.84 (m, 2H), 3.81 (dd, 1H), 3.68-3.55 (m, 4H), 3.15-3.05 (m, 1H), 3.02-2.99 (m, 6H), 2.82 (dd, 1H), 2.38-2.26 (m, 2H), 2.10-1.98 (m, 4H), 1.96-1.55 (m, 4H), 1.49-1.42 (m, 4H), 1.41-1.30 (m, 6H), 1.26-1.12 (m, 4H).

### Structures and Names

### Rivaroxaban

### 5-Chloro-N-({(5S)-2-oxo-3-[4-(3-oxo-4-morpholinyl)phenyl]-1,3-oxazolidin-5-yl}-methyl)-2-thiophenecarboxamide

### Described in WO 01/047919 (Example 44)

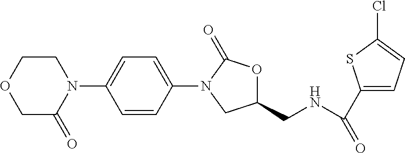 [SMILES: O=C(NC[C@H]1CN(c2ccc(N3CCOCC3=O)cc2)C(=O)O1)c1ccc(Cl)s1]

### SATI

### 5-Chloro-N-{[(5S)-3-{4-[3-{2-[(trans-4-hydroxycyclohexyl)amino]ethyl}-2-oxopyridin-1(2H)-yl]-3,5-dimethylphenyl}-2-oxo-1,3-oxazolidin-5-yl]methyl}-thiophene-2-carboxamide

### Described in SATI in WO 2008/155032 (Example 38)

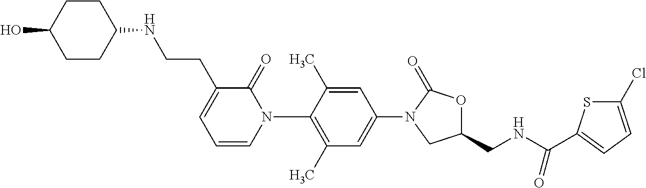

### Example 2

### Antibody Generation from n-CoDeR Libraries

### Phage Selections:

The isolation of human antibodies or antigen binding fragments thereof against FXa inhibitors comprising a group of formula 1 was performed by phage display technology employing the naive Fab antibody library n-CoDeR of BioInvent International AB (Lund, Sweden; described in Soderling et al., Nat. Biotech. 2000, 18:853-856), which is a Fab library in which all six CDRs are diversified.

Standard buffers used in this example are:
1×PBS: from Sigma (D5652-501)
PBST: 1×PBS supplemented with 0.05% Tween20 (Sigma, P7949)
PBST-MP3%: PBST supplemented with 3% milkpowder (Cell Signaling, 9999)

Briefly, an aliquot of the Fab antibody library was depleted for unwanted binders by sequential pre-incubation on an end-to-end rotator first with streptavidin-coated Dynabeads M280 (Invitrogen, 11206D) for 60 min and then with FITC-biotin (Sigma, B8889) at a concentration of 500 nM for 10 min. Subsequently, 150 μl fresh streptavidin-coated beads were pre-coupled to either 500 nM compound Example 1K or 500 nM compound Example 1L in selection buffer PBST during a 1.5 h incubation step followed by extensive washing of the beads with PBST. Then coated beads were blocked by incubating in blocking buffer for 30 min on an end-to-end rotator. Coated and blocked beads were washed extensively with blocking buffer and then mixed with blocked and depleted aliquots of the Fab-library. After 60 min incubation on an end-to-end rotator the samples were washed 3 times with blocking buffer followed by 3 times washing with PBST, and 3 final washing steps in PBS. Bound phages were eluted by adding 400 μl trypsin solution (1 mg/ml in PBS; Sigma, T1426). After 30 min incubation at r.t., 40 μl aprotinin (2 mg/ml in PBS; Sigma, A1153) were added to stop trypsin digestion.

Eluted phages were propagated and phage titers determined as previously described (Cicortas Gunnarsson et al., Protein Eng Des Sel 2004; 17 (3): 213-21). Briefly, aliquots of the eluate solution were saved for titration experiments while the rest was used to transform exponentially growing E. coli HB101′ (from Bioinvent) for preparation of new phage stocks used in a second and a third selection round employing 100 nM and 20 nM of target molecules, respectively. For each selection round, both input and output phages were titrated on exponentially growing E. coli HB101′ and clones were picked from round 2 and 3 for analysis in Phage ELISA.

### Enzyme-Linked Immunosorbent Assay (ELISA):

### Phage ELISA:

Selected phages from different selection rounds were analyzed for specificity using phage ELISA. Briefly, phage expression was performed by adding 10 μl of over night culture (in LB-medium supplemented with 100 μg/ml ampicillin (Sigma, A5354) and 15 μg/ml tetracycline (Sigma, T3383)) to 100 μl fresh medium (LB-medium supplemented with 100 μg/ml ampicillin, 15 μg/ml tetracyclin and 0.1% glucose (Sigma, G8769) and shaking at 250 rpm and 37° C. in 96-well MTP until an OD600 of 0.5 was reached. Subsequently helper phage M13KO7 (Invitrogen, 420311) was added and samples were incubated for another 15 min at 37° C. without shaking. After addition of IPTG (f.c. of 0.25 mM) cells were incubated over night at 30° C. while shaking at 200 rpm.

96-well ELISA-plates precoated with streptavidin (Pierce, 15500) were coated over night at 4° C. with 1 μg/ml compounds from Examples 1K and 1L, respectively. The next day plates were washed 3 times with PBST, treated with blocking reagent, and washed again 3 times with PBST. After that 50 μl aliquots from phage expressions were transferred per well and incubated for 1 h at r.t. After washing 3 times with PBST, anti M13 antibody coupled to HRP (GE Healthcare, 27-9421-01; 1:2500 diluted in PBST) was added and incubated for 1 h at r.t. Color reaction was developed by addition of 50 μl TMB (Invitrogen, 2023) and stopped after 5-15 mM by adding 50 μl H2SO₄ (Merck, 1120801000). Colorimetric reaction was recorded at 450 nM in a plate reader (Tecan).

Screening of sFabs by ELISA:

For the generation of soluble Fab fragements (sFabs) phagemid DNA from the selection rounds 2 and 3 was isolated and digested with restriction enzymes EagI (Fermentas, FD0334) and EcoRI (NEB, R0101L) according to the providers instructions in order to remove the gene III sequence. The resulting fragment was re-ligated and constructs were transformed into chemically competent E. coli Top10 using standard methods. Single clones were picked, transferred to 96-well plates containing LB-media (100 μg/ml, 0.1% glucose) and shaken at 250 rpm and 37° C. until an OD600 of 0.5 was reached. After that sFab production was induced by the addition of IPTG (f.c. 0.5 mM) and incubation was continued over night at 30° C. while shaking at 200 rpm. Next morning BEL-buffer (24.7 g/l boric acid; 18.7 g/l NaCl; 1.49 g/l EDTA pH 8.0; 2.5 mg/ml lysozyme (Roche)) was added to each well and 50 μl of the treated cultures were analyzed for binding of sFabs to the target in an ELISA essentially as described for phages, except that detection was performed with an anti-hIgG (Fab-specific) coupled to HRP (Sigma; A 0293).

### Example 3

### Small-Scale Production of Soluble Fab Screening Hits

Unique screening hits were produced in small scale for the initial characterization in surface plasmon resonance and functional neutralization of rivaroxaban in a biochemical FXa activity assay. 50 to 100 ml of LB-medium (supplemented with 0.1 mg/ml ampicillin and 0.1% glucose) were inoculated with a pre-culture of the respective E. coli Top 10 clone, containing a unique Fab sequence cloned into the initial pBIF-vector but lacking the gene III sequence. Production of sFabs was induced by the addition of 0.5 mM IPTG (final concentration) and incubation was continued over night at 30° C. at 250 rpm shaking.

Subsequently, cells were harvested by centrifugation and gently lysed by 1 h incubation at 4° C. in a lysis buffer, containing 20% sucrose (w/v), 30 mM TRIS, 1 mM EDTA, pH 8.0, 1 mg/ml lysozyme (Sigma L-6876) and 2.5 U/ml Benzonase (Sigma E1014). The cleared supernatant was then applied to a capture select lambda affinity matrix (BAC 0849.010). After washing of the matrix with PBS, bound sFabs were eluted with 100 mM glycin/HCl, pH 3 and immediately neutralized with 1 M HEPES-buffer. Samples were subsequently dialysed against PBS and submitted to a second purification step on His-Multi-Trap plates (GE) according to manufacturer's instructions. Eluted sFabs were dialysed against PBS and analysed for protein content and for purity by SDS-PAGE.

### Example 4

### Functional Neutralization of Rivaroxaban in a Biochemical Factor Xa Activity Assay

Factor Xa activity was inhibited by rivaroxaban to 20-30% remaining FXa activity, and neutralization of this inhibition by test compounds (e.g. Fab fragments) was analyzed:

Serial dilutions of test compounds in assay buffer (50 mM HEPES pH 7.8, 250 mM NaCl, 6 mM CaCl₂, 0.01% Brij35, 1 mM glutathione, 4 mM EDTA, 0.05% bovine serum albumin) were performed (typical concentrations ranging from 5 μM to 0.0007 μM).

20 μL of the diluted test compounds were placed in 384 well microtiter plates (Greiner, Frickenhausen, Germany), followed by the addition of 10 μL of a 1:400 dilution (250 μM) of the FXa substrate Pefafluor Xa (100 mM in DMSO, Loxo, Dossenheim, Germany) in assay buffer. The enzymatic reaction was started by addition of 20 μL of a factor Xa (HTI, Essex Junction, VT USA) dilution in assay buffer containing the factor Xa inhibitor rivaroxaban. Simultaneously, control reactions without rivaroxaban were started.

During incubation at 32° C., reaction progress curves were monitored using a fluorescence microtiter plate reader (e.g Tecan Ultra Evolution, Tecan Group Ltd., Mannedorf, Switzerland; excitation 360 nm, emission 465 nm).

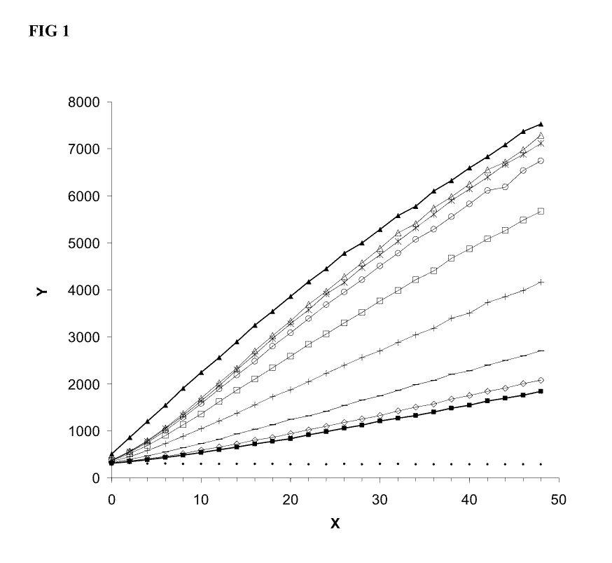

The dilution of FXa was chosen that in the control reactions the reaction kinetics was linear, and less than 50% of the substrate was consumed (typical final FXa concentration in the assay: 0.05 nM). The concentration of rivaroxaban was chosen that FXa activity was inhibited by 70-80%, compared to the control reactions (typical final concentration of rivaroxaban in the assay: 0.6 nM). Results are depicted in FIG. 1.

EC50 values were determined by plotting the test compound concentration against the percentage of factor Xa activity after 50 min incubation time. EC₅₀ values were defined as the concentration of test compound reversing 50% of the rivaroxaban induced FXa inhibition.

### Example 5

### Determination of Affinities by Surface Plasmon Resonance (Biacore)

Binding affinities of Fab-fragments were determined by surface plasmon resonance analysis on a Biacore T100 instrument (GE Healthcare Biacore, Inc.). Fab fragments were diluted to a final concentration of 10 μg/ml in 10 mM sodium acetate, pH 4.5, and immobilized on a CM5 chip (GE Healthcare Biacore, Inc.) at levels of 3000-5000RU by amine-coupling chemistry for flow cells 2, 3 and 4, respectively. Flow cell 1 was used as a reference. Various concentrations of analyte, rivaroxaban, compound from Example 1G, SATI (described in WO 2008/155032 (Example 38)), apixaban (described in WO2003/026652 (Example 18)), edoxaban (described in WO2003/000680 (Example 192), US 2005 0020645 (Example 192)), razaxaban (described in WO1998/057951 (Example 34)) respectively (200 nM, 100 nM, 50 nM, 25 nM, 12.5 nM, 6.25 nM, 3.12 nM, and 1.56 nM) in HEPES-EP buffer (GE Healthcare Biacore, Inc.) were injected over immobilized Fab fragments at a flow rate of 60 μl/min for 3 minutes and the dissociation was allowed for 10 minutes. Sensograms were generated after in-line reference cell correction followed by buffer sample subtraction. The dissociation equilibrium constant (K_{D}) was calculated based on the ratio of association and dissociation rated constants, obtained by fitting sensograms with a first order 1:1 binding model using BiaEvaluation Software. Data is summarized in Table 6 and 7.

TABLE 6
Characterization of some initial Fab-hits from panning/screening and
the optimized variant M18-G08-G-DKTHT: Summary of affinity data
(K_{D} in nM) of immobilized Fabs for rivaroxaban from
SPR-analysis (Biacore) and of half maximal effective
concentration (EC₅₀ in μM) of Fabs in biochemical FXa-
assay (0.05 nM FXa) in the presence of rivaroxaban (0.6 nM).

| Fab | K_{D} (nM) | EC₅₀ (μM) |
|---|---|---|
| M16-D05 | <500 | <1 |
| M14-G07 | <50 | <0.5 |
| M15-B07 | <500 | <1 |
| M25-E05 | <500 | <1 |
| M18-A10 | <500 | <1 |
| M16-A03 | <500 | <2 |
| M18-G08 | <50 | <0.5 |
| M18-G08-G-DKTHT | <10 | <0.01 |

TABLE 7
Characterization of selected Fabs for binding to various
FXa-inhibitors: Summary of affinity data (K_{D} in nM)
of immobilized Fabs for rivaroxaban from SPR-analysis
(Biacore).

| | Rivar- | Example | | | Edoxa- | Razax- |
|---|---|---|---|---|---|---|
| Fab | oxaban | 1G | SATI | Apixaban | ban | aban |
| M14-G07 | <50 | >1000. | n.b. | n.b. | n.b. | n.b. |
| M18-G08 | <50 | <50 | <50 | n.b. | n.b. | n.b. |
| M18-G08- | <10 | n.t. | <10 | n.b. | n.b. | n.b. |
| G-DKTHT | | | | | | |

n.b.: no binding
n.t.: not tested

### Example 6

### Affinity Determination by Isothermal Titration Calorimetry (ITC)

For determination of thermodynamic parameters a VP-ITC Isothermal

Titration calorimeter with control and analysis software (Microcal/GE Healthcare, Freiburg, Germany) was applied. Here, Isothermal Titration calorimetry was used to determine the order of the association constant of a test compound (e.g. Fab fragment) binding to rivaroxaban in solution.

A 10 mM solution of rivaroxaban (Bayer Healthcare, Wuppertal, Germany) in DMSO was diluted 1:2000 in PBS buffer (pH 7.4, Sigma, Taufkirchen, Germany). The solution was degassed and filled into the sample cell (1.4 mL). The reference cell was filled with water. A 50 μM solution of the test compound in PBS buffer was prepared. The DMSO concentration in the test compound solution was adjusted to the DMSO concentration in the sample cell. After degassing, the test compound solution was drawn into the instrument's syringe.

At constant temperature (25° C.) and providing continuous mixing, the test compound solution was injected into the sample cell, making use of the instrument's control software (Reference Power: 5 μcal/s, twelve injections 10 μL each, duration of each injection 20 s, waiting time between each injection 300 s). Heat released during the binding reaction was monitored over time and data were analyzed using the analysis software. For M18-G08-G-DKTHT a K_{D} of <1 nM for rivaroxaban was estimated from the titration curve.

### Example 7

### Determination of the K_{D} Value of Fab M18-G08-G-DKTHT Towards Rivaroxaban in Dulbecos PBS

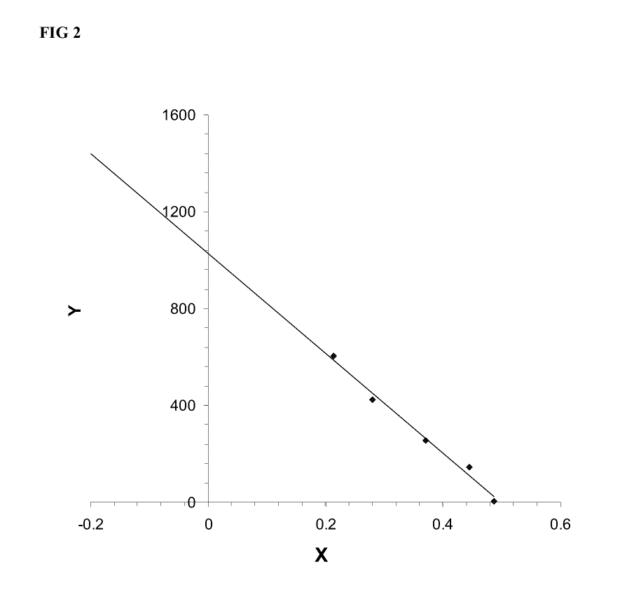

The determination of the unbound concentration of rivaroxaban in the presence of M18-G08-G-DKTHT allows the determination of the K_{D} value of the Fab towards rivaroxaban in solution. The K_{D} value was calculated using the Rosenthal-Scatchard plot (FIG. 2).

Rivaroxaban was incubated at concentrations of 0.214 μM to 0.583 μM with 0.5 μM Fab M18-G08-G-DKTHT at room temperature for 20 mM in Dulbeccos PBS (DPBS) buffer. The solution was than added to an ultrafiltration device containing a membrane with an exclusion size of 30000 Da. Samples were centrifuged for 3 min at 100 g. 50 μL of the ultrafiltrate and start solution was spiked with 150 μL of a solution of ammonium acetate/acetonitril (1/1 v/v) pH 3.0 containing the internal standard. Samples were analyzed by LC-MS/MS using an API 4000 (AB Sciex). The fu (=fraction unbound) values were calculated according to the relation fu (%)=concentration filtrate/(concentration start solution*100) and were corrected for unspecific binding to the ultrafiltration device as described before (Schuhmacher J. et al., J Pharm Sci. 2004; 93(4):816-30). A K_{D} value of about 0.5 nM was calculated from the slope of the Rosenthal Scatchard Plot (FIG. 2).

### Example 8

### Reversal of the Effect of Rivaroxaban or SATI in the Thrombin Generation Assay by Fab-Antidote

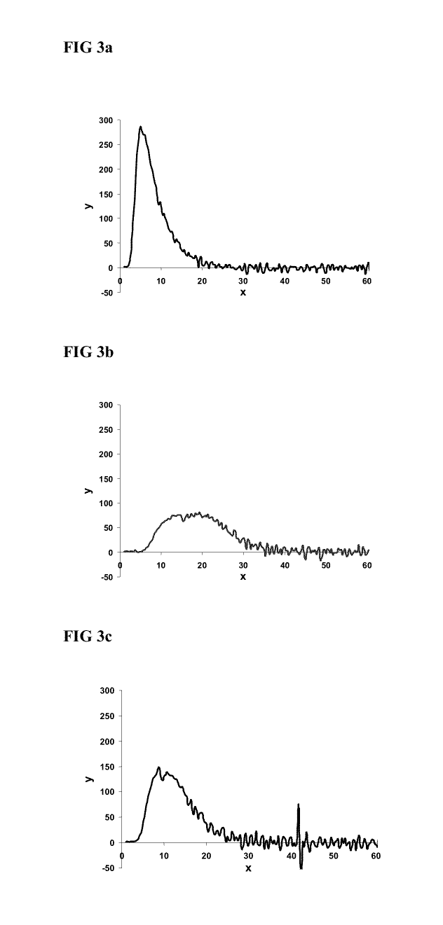

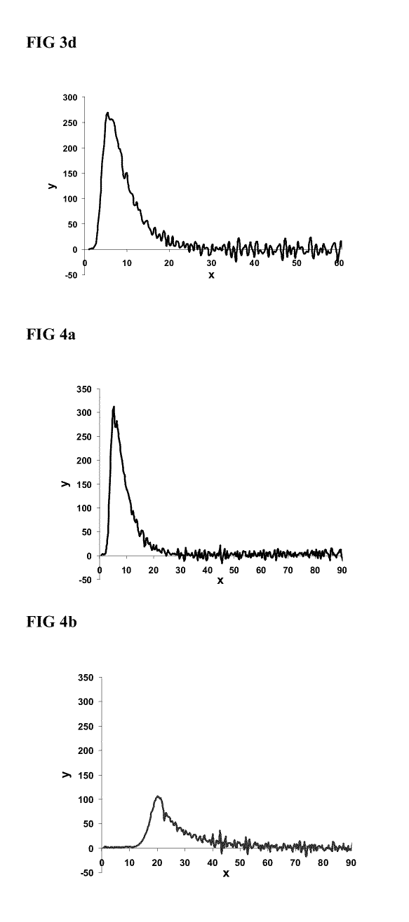

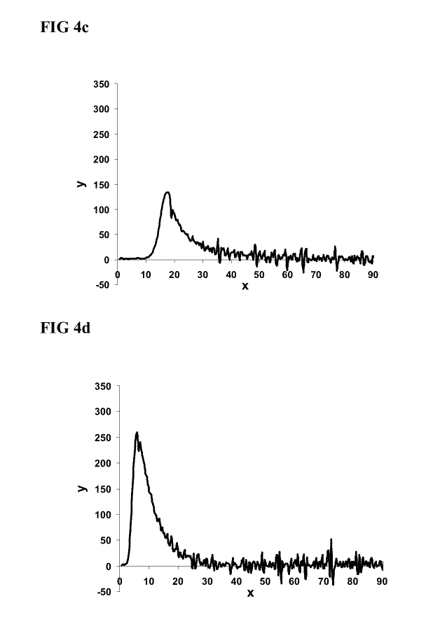

The thrombin generation assay according to Hemker allows to investigate the effects of compounds on the kinetics of the coagulation cascade. Tissue factor and Ca²⁺ are added to human platelet poor plasma to initiate the extrinsic pathway, and the activity of thrombin generated is determined with a specific, fluorescently labeled substrate (Bachem, I-1140 (Z-Gly-Gly-Arg-AMC)). The reaction was performed in 20 mM Hepes, 60 mg/ml BSA, 102 mM CaCl₂, pH 7.5 at 37° C. Reagents to start the reaction and a thrombin calibrator are commercially available from Thrombinoscope. Measurements are carried out in a Thermo Electron Fluorometer (Fluoroskan Ascent) equipped with a 390/460 nm filter set and a dispenser. All experimental steps are carried out according to the manufacturer's instructions (Thrombinoscope). Inhibitor (rivaroxaban or SATI, 0.1 μM) and antidote, when present, were preincubated with plasma for 5 min at 37° C. before initiation of thrombin generation. M18-G08-G-DKTHT concentration-dependently neutralizes the effect of rivaroxaban and SATI as shown in FIGS. 3 and 4, respectively. FIG. 5 demonstrates that increasing concentrations of the Fab M18-G08-G-DKTHT itself do not modify the thrombogram, underlining that the Fab has no intrinsic influence on coagulation.

### Example 9

### Reversal of Rivaroxaban's Effect in a FXa Activity Assay in Plasma

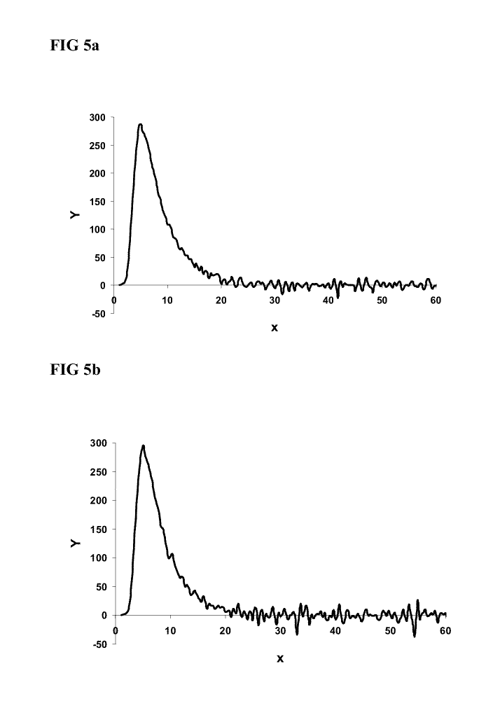

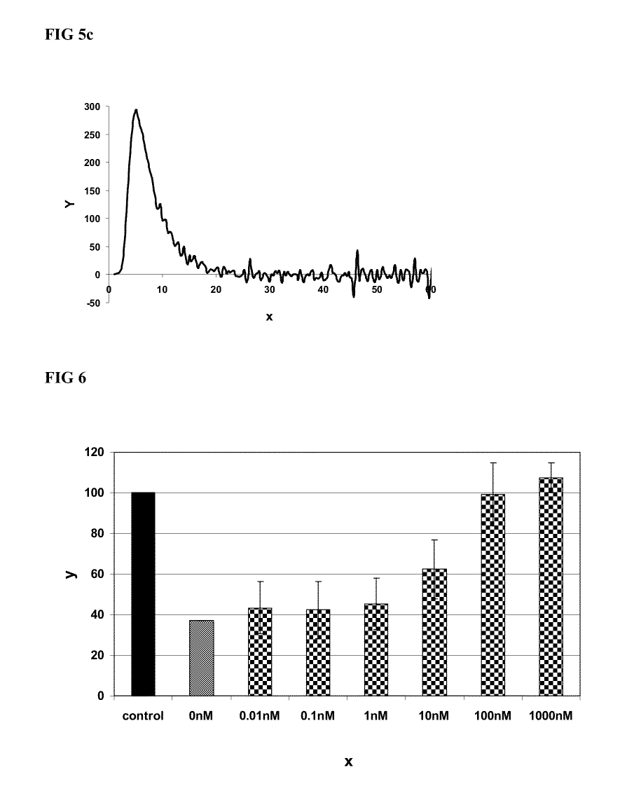

In order to investigate the inhibition of FXa activity in plasma by rivaroxaban and reversal of its inhibitory effect, citrated human plasma (Octapharm) is incubated with rivaroxaban diluted in Hirudin and incubated for 3 min at 37° C. Then the Fab is added and after 5 min incubation at 37° C. FX activation is started by adding Russel's Viper Venom (RVV-X, Pentapharm, final concentration 5 mU/ml) in buffer containing 0.1 mM calcium. FXa activity is determined by measuring the cleavage of a specific, fluorogenically-labeled substrate (Bachem, I-1100, concentration 50 μM) and the flourescence was monitored continuously at 360/465 nm using a SpectraFlourplus Reader (Tecan). In FIG. 6 the effect of rivaroxaban on FXa activity in plasma and reversal of the inhibitory effect by increasing concentrations of the Fab M018-G08-G-DKTHT is shown.

### Example 10

### Reversal of Rivaroxaban's Effect on Prothrombin Time (PT) in Vitro

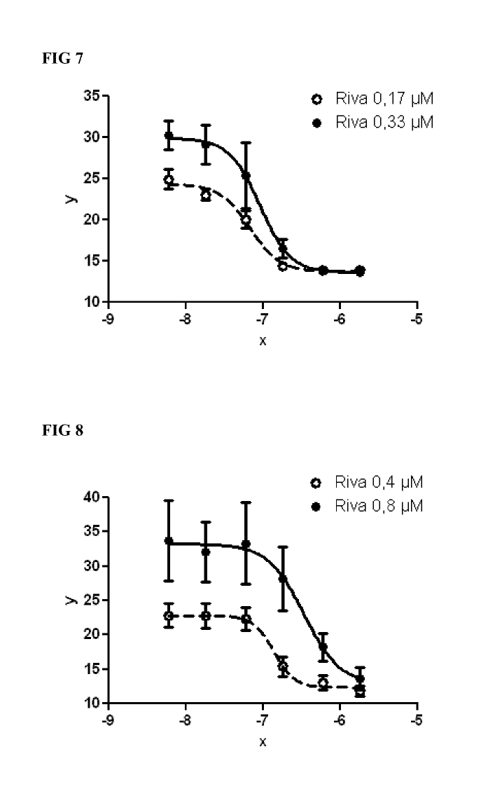

Citrated blood (0.11 M Na-citrate/blood, 1:9 v/v) was obtained from human donors by venipuncture or from anesthetized Wistar rats (Charles River) by aortic cannulation and centrifuged at 4000 g for 15 minutes for separation of platelet-poor plasma. Plasma samples were mixed with rivaroxaban (concentrations as in FIGS. 7 and 8, dissolved in DMSO, final DMSO concentration 1%) and incubated for 10 minutes at room temperature. Antidote was added to the Plasma-rivaroxaban mixture and incubated for another 10 minutes at room temperature. The PT assay was run using Recombiplastin (Instrumentation Laboratory) as tissue factor source on an AMAX 200 automated coagulometer (Trinity Biotech) according to manufacturer's instructions. The composition of the final assay volume is ⅓ plasma and ⅔ PT reagent. IC50 values were calculated for the antidote concentration required for half-maximal normalization of the PT prolongation produced by the respective rivaroxaban concentration. Data are given as means±sem from 5 experiments and represent final assay concentrations (Table 8 and FIGS. 7 and 8).

TABLE 8
Effects of M18-G08-G-DKTHT on Prothrombin Time (PT) in vitro

| | Rivaroxaban | PT prolongation | IC₅₀ M18-G08-G- |
|---|---|---|---|
| | concentration | by rivaroxaban (x | DKTHT |
| Plasma species | (μM) | control) | (μM) |
| Human | 0.17 | 1.8 | 0.07 ± 0.01 |
| | 0.33 | 2.2 | 0.09 ± 0.02 |
| Rat | 0.4 | 1.9 | 0.14 ± 0.03 |
| | 0.8 | 2.6 | 0.34 ± 0.17 |

### Example 11

### Cloning, Expression and Quantification of Expression Levels of Antibody Variants

The heavy and light chain of the two rivaroxaban binding Fabs M14-G07 and M18-G08 which both carry a c-myc-tag and a hexa-histidine tag at the C-terminus of the heavy chain were subcloned into the pET28a bacterial expression vector (Novagen/Merck Chemicals Ltd., Nottingham, UK) and transformed into Top10F′ cells (Invitrogen GmbH, Karlsruhe, Germany). Mutations were introduced by standard oligo-based site-directed mutagenesis and confirmed by DNA sequencing.

For Fab antibody expression, variant plasmids were transformed into the T7 Express lysY/Iq Escherichia coli strain (New England Biolabs, C3013), inoculated into an overnight culture in LB medium including kanamycin (30 μg/ml) and incubated at 37° C. for 18 hours. Expression cultures were generated by transferring 5% of the overnight culture into fresh LB medium with kanamycin (30 μg/ml). After 6 hours, 1 mM isopropyl-b-D-1-thiogalactopyranoside (Roth, 2316.5) was added to induce Fab expression and the cultures were incubated for additional 18 hours at 30° C.

For quantification of expression levels an ELISA approach was used. Briefly, MTP plates (Nunc Maxisorp black, 460518) were incubated with a Fab-specific antibody (Sigma, I5260) diluted in coating buffer (Candor Bioscience GmbH, 121500) at 4° C. over night, washed three times with PBST (phosphate buffered saline: 137 mM NaCl Merck 1.06404.5000; 2.7 mM KCl Merck 1.04936.1000; 10 mM Na₂HPO₄ Merck 1.06586.2500, 1.8 mM KH₂PO₄ Merck 1.04871.5000; containing 0.05% Tween 20 Acros Organics, 233360010), blocked with 100% Smart Block (Candor Bioscience GmbH, 113500) for 1 h at room temperature and washed again. Cultures were diluted in 10% Smart Block in PBST and bound to the MTP plates for 1 h at room temperature. After washing with PBST, captured Fabs were incubated with a HRP (horseradish peroxidase)-coupled anti c-myc antibody (Bethyl Laboratories Inc., A190-105P), washed and incubated with 10 μM Amplex Red substrate (Invitrogen, A12222) for 10 to 30 minutes at room temperature in the dark followed by fluorescence measurement. Measured quantification signals were filtered according to the dynamic range as determined by a dilution series of a purified Fab control.

### Example 12

### Determination of Activity of Antibody Variants Using an ELISA-Based Assay

To determine the activity of the mutated antibody variants on compound from Example 1K an equilibrium or dissociation limited ELISA assay format was used. Briefly, MTP plates (Nunc Maxisorp black, 460518) were coated with 4 μg/ml streptavidin (Calbiochem, 189730) diluted in coating buffer (Candor Bioscience GmbH, 121500) and incubated over night at 4° C. After washing with PBST, plates were blocked with 100% Smart Block (Candor Bioscience GmbH, 113500) in PBST for 1 h at room temperature and the washing step was repeated. Plates were incubated with compound from Example 1K at varying concentrations (0.3-200 nM) for 1 h at 37° C., washed with PBST and antibody fragments were immobilized by adding 25 μl of the crude bacterial cultures for 1 h at room temperature. After washing with PBST a competition step or the immediate detection was performed. For the competition 300 nM rivaroxaban diluted in 10% Smart Block in PBST were added and incubated for 1.5-3 h at room temperature. For the detection of the residually bound Fabs a HRP-coupled anti-lambda antibody (Sigma, A5175) diluted in 10% Smart Block in PBST was added for 1 h at room temperature. After washing 10 μM Amplex Red (Invitrogen, A12222) were added and incubated for 10 to 30 min at room temperature followed by measurement of the fluorescence signal.

### Example 13

### Analysis of Variant Performance in a FXa De-Inhibition Assay (FXa DIA)

To determine the activity of wild-type (wt) and mutated Fab variants on unmodified rivaroxaban a de-inhibition assay of FXa activity was performed.

Briefly, 10 μl of crude bacterial cultures were incubated with 1 μl 200 nM rivaroxaban and 2 μl of FXa substrate (Fluophen, Hyphen BioMed, 329011) for 1 h at room temperature in black low volume plates (Greiner, 784076). Then, 7 μl of 28 nM FXa (Haematologic Technologies Inc., HCXA-0060) diluted in assay buffer (20 mM Tris, Merck 1.08382.2500; 100 mM NaCl, Merck 1.06404.5000; 2.5 mM CaCl₂*2H₂O, Merck 1.02382.1000; 0.1% bovine serum albumin, Sigma A4503; 0.1% PEG 8000, Sigma P2139) were added and enzyme activity was recorded over time by measuring the fluorescence signal at 440 nm using a micro plate reader e.g. Tecan Infinite F500. The fluorescence signal was integrated over time and ratios of variant to wild-type were compared.

### Example 14

### Single and Multiple Amino Acid Substitutions

Provided in Table 9 are several examples of single and/or double amino acid substitutions introduced into the heavy and/or the light chain of M14-G07 (wt). Performance of the variants was analyzed in quadruples in the ELISA without a competition step and the FXa deinhibition assay (FXa DIA). In the ELISA, averages were calculated and normalized to the respective average expression level. Overall performance of variants was evaluated by comparing the variant to wt ratio from 2-3 independent experiments. Variants with an average ratio above wt plus 2×SD (standard deviation of the ratio) were considered as improved and are marked with “++”, whereas variants with a ratio below wt minus 2×SD were considered as reduced in their binding affinity and are marked with “−”. All variants with a performance in between both thresholds are marked with “+/−”. Variants with average fluorescence counts below the negative control (non-expressing cells) plus 3×SD were considered as non-binding and marked with “−−” with none of the variants fulfilling this criteria. In the FXa deinhibition assay averages were calculated and overall performance of variants was evaluated by comparing the variant to wt ratio from 2-3 independent experiments. Variants with an average ratio above wt plus 2×SD were considered as improved and are marked with “++”, whereas variants with a ratio below wt minus 2×SD were considered as either reduced in their binding affinity or non-binding and are marked with “−−”. All variants with a performance in between both thresholds are marked with “+/−”. Variants not analyzed are marked with “nd” (not determined). CDRs were defined according to Kabat.

TABLE 9
Analysis of single and double amino acid substitutions
within M14-G07.

| variable domain | M14-G07 | | FXa |
|---|---|---|---|
| location | mutation | ELISA | DIA |
| FR1 | HC_T28K | +/− | +/− |
| FR1 | HC_T28R | +/− | +/− |
| FR1 | HC_G30S | +/− | Nd |
| CDR H1 | HC_D31A | +/− | +/− |
| CDR H1 | HC_D31R | +/− | +/− |
| CDR H1 | HC_D31S | +/− | Nd |
| CDR H1 | HC_A33G | +/− | +/− |
| CDR H1 | HC_S35A | ++ | +/− |
| CDR H1 | HC_S35G | +/− | +/− |
| FR2 | HC_S49G | +/− | +/− |
| CDR H2 | HC_G50A | − | Nd |
| CDR H2 | HC_G53R | ++ | +/− |
| CDR H2 | HC_S57I | +/− | +/− |
| CDR H2 | HC_S57K | +/− | +/− |
| CDR H2 | HC_S57M | +/− | +/− |
| CDR H2 | HC_S57R | +/− | +/− |
| CDR H2 | HC_T58R | +/− | +/− |
| FR3 | HC_A97Q | − | −− |
| FR3 | HC_R98K | − | Nd |
| CDR H3 | HC_E99Q | +/− | +/− |
| CDR H3 | HC_G100A | +/− | +/− |
| CDR H3 | HC_G100S | +/− | ++ |
| CDR H3 | HC_G100V | − | +/− |
| CDR H3 | HC_E101G | − | +/− |
| CDR H3 | HC_E101R | +/− | +/− |
| CDR H3 | HC_E101S | +/− | +/− |
| CDR H3 | HC_T102D | +/− | +/− |
| CDR H3 | HC_T102F | +/− | +/− |
| CDR H3 | HC_T102L | +/− | +/− |
| CDR H3 | HC_T102R | +/− | +/− |
| CDR H3 | HC_G105Y | +/− | Nd |
| CDR H3 | HC_L106F | − | Nd |
| CDR H3 | HC_V108A | +/− | +/− |
| CDR H3 | HC_V108C | +/− | +/− |
| CDR H3 | HC_V108W | − | +/− |
| CDR H3 | HC_V108Y | +/− | Nd |
| FR4 | HC_T118S | +/− | Nd |
| FR1 | LC_Q1E | − | Nd |
| CDR L1 | LC_S23K | +/− | ++ |
| CDR L1 | LC_S23T | +/− | +/− |
| CDR L1 | LC_S25N | +/− | +/− |
| CDR L1 | LC_S25V | +/− | +/− |
| CDR L1 | LC_S26A | +/− | +/− |
| CDR L1 | LC_S27A | +/− | +/− |
| CDR L1 | LC_S27R | +/− | +/− |
| CDR L1 | LC_N28S | +/− | ++ |
| CDR L1 | LC_S31A | +/− | ++ |
| CDR L1 | LC_N32F | +/− | +/− |
| CDR L1 | LC_N32G | +/− | +/− |
| CDR L1 | LC_N32Y | ++ | ++ |
| CDR L1 | LC_Y33L | +/− | +/− |
| CDR L1 | LC_V34G | − | +/− |
| CDR L1 | LC_V34S | +/− | +/− |
| FR2 | LC_L48V | +/− | +/− |
| FR2 | LC_Y50V | +/− | +/− |
| CDR L2 | LC_D51R | − | Nd |
| CDR L2 | LC_N53A | − | +/− |
| CDR L2 | LC_N53P | +/− | ++ |
| CDR L2 | LC_N53R | ++ | +/− |
| CDR L2 | LC_N53S | +/− | ++ |
| CDR L2 | LC_N53V | +/− | +/− |
| CDR L2 | LC_D54Q | +/− | Nd |
| CDR L2 | LC_R55L | +/− | +/− |
| CDR L2 | LC_P56S | +/− | +/− |
| CDR L2 | LC_S57W | ++ | +/− |
| FR3 | LC_G58E | ++ | +/− |
| CDR L3 | LC_V90A | − | Nd |
| CDR L3 | LC_V90N | +/− | +/− |
| CDR L3 | LC_D93E | +/− | ++ |
| CDR L3 | LC_D94C | ++ | +/− |
| CDR L3 | LC_D94V | +/− | +/− |
| CDR L3 | LC_D94W | ++ | +/− |
| CDR L3 | LC_S95V | +/− | +/− |
| CDR L3 | LC_L96G | +/− | +/− |
| CDR L3 | LC_L96W | ++ | +/− |
| CDR L3 | LC_L96Y | +/− | +/− |
| CDR L3 | LC_N97S | +/− | Nd |
| CDR L3 | LC_G98L | − | +/− |
| CDR L3 | LC_G98T | − | Nd |
| CDR L3 | LC_H99K | − | +/− |
| CDR L3 | LC_H99P | − | Nd |
| CDR L3 | LC_H99T | +/− | +/− |
| CDR L3 | LC_W100V | − | Nd |
| CDR L3 | LC_V101F | +/− | +/− |
| CDR L3 | LC_V101P | +/− | +/− |
| CDR L3 | LC_V101W | +/− | ++ |
| HC_FR1_LC_CDR L1 | HC_A23T_LC_N32W | +/− | +/− |
| HC_FR3_HC_CDR H3 | HC_A92S_HC_T102C | − | +/− |
| HC_FR1_LC_CDR L3 | HC_G10S_LC_G98A | − | +/− |
| HC_FR1_LC_CDR L2 | HC_L11M_LC_S57Y | +/− | ++ |
| HC_FR1_LC_CDR L1 | HC_L5M_LC_S26L | − | +/− |
| HC_CDR H2_HC_CDR H2 | HC_S57T_HC_T58V | +/− | +/− |
| LC_CDR L1_LC_CDR L1 | LC_S25G_LC_N32Y | ++ | +/− |

Provided in Table 10 are examples of combined amino acid substitutions within M14-G07 antibodies. While not every combination is provided in Table 10, it is contemplated that the anti-rivaroxaban antibody may comprise any combination of modifications provided. Variant performance was analyzed in quadruples in the ELISA without a competition step. Averages were calculated, average background signals determined on a streptavidin coated plate without compound from Example 1K were subtracted if the compound from Example 1K concentration used for coating was below 10 nM and signals were normalized to the respective average expression level. Overall performance of variants was evaluated by comparing the variant to reference ratio from 2-3 independent experiments using a 2-fold improved reference variant as compared to wt. Variants with an average ratio above reference plus 2×SD are marked with “+++”, whereas variants with a ratio below reference minus 2×SD are marked with “+/−”. All variants with a performance in between both thresholds are marked with “++”. Variants with a ratio below 0.5 are marked with “−” with none of the variants fulfilling this criteria. CDRs were defined according to Kabat.

TABLE 10
Example of multiple amino acid substitutions within M14-G07.

| | HC | | | | | | | | | LC | | | | | | | | | | | | |
|---|---|---|---|---|---|---|---|---|---|---|---|---|---|---|---|---|---|---|---|---|---|---|
| M14-G07-X; | FR1 | FR1 | CDR H1 | FR2 | | CDR H3 | | | FR4 | CDR L1 | | | | | FR2 | CDR L2 | | CDR L3 | | | | |
| X = | T28 | G30 | D31 | V37 | S49 | E99 | E101 | G105 | T118 | S23 | S26 | S27 | S31 | N32 | Q38 | N53 | D54 | D94 | L96 | N97 | H99 | ELISA |
| 1 | R | S | S | | G | Q | R | Y | S | | | R | | F | | | Q | | W | S | T | +++ |
| 2 | | S | S | | G | Q | R | Y | S | C | | R | | Y | | | Q | | W | S | T | +++ |
| 3 | | S | S | | | Q | R | Y | S | | | R | | Y | | R | Q | | W | S | T | +++ |
| 4 | R | S | S | | G | Q | R | Y | S | | | R | A | Y | | R | Q | | W | S | T | +++ |
| 5 | R | S | S | | | Q | R | Y | S | | | R | | Y | | R | Q | | W | S | T | +++ |
| 6 | R | S | S | I | G | Q | R | Y | S | | | R | | Y | | R | Q | | W | S | T | +++ |
| 7 | R | S | S | | G | Q | R | Y | S | | | R | | Y | | | Q | | W | S | | +++ |
| 8 | | S | S | | G | Q | R | Y | S | | | R | A | F | | R | Q | | W | S | T | +++ |
| 9 | R | S | S | | G | Q | R | Y | S | | | R | | F | | | Q | V | | S | | +++ |
| 10 | | S | S | | G | Q | R | Y | S | | | R | | F | | | Q | | W | S | | +++ |
| 11 | R | S | S | | G | Q | R | Y | S | | | R | A | F | | R | Q | | W | S | | +++ |
| 12 | | S | S | | | Q | R | Y | S | | | R | | Y | | | Q | | W | S | T | +++ |
| 13 | | S | S | | G | Q | R | Y | S | | | R | | F | | R | Q | | W | S | T | +++ |
| 14 | | S | S | | G | Q | R | Y | S | | | R | | Y | | R | Q | | Y | S | T | +++ |
| 15 | | S | S | | G | Q | R | Y | S | | | | | Y | | | Q | | W | S | | +++ |
| 16 | R | S | S | | G | Q | R | Y | S | | | R | | F | | R | Q | | Y | S | T | +++ |
| 17 | | S | S | | G | Q | R | Y | S | | | R | A | F | | R | Q | | W | S | T | +++ |
| 18 | R | S | S | | G | Q | R | Y | S | | | R | | Y | | | Q | | W | S | T | +++ |
| 19 | | S | S | | | Q | R | Y | S | | | R | | F | | R | Q | | | S | | +++ |
| 20 | R | S | S | | G | Q | R | Y | S | | | | A | Y | | | Q | | | S | T | +++ |
| 21 | R | S | S | | G | Q | | Y | S | | | | | Y | H | | Q | | W | S | T | +++ |
| 22 | | S | S | | G | Q | R | Y | S | | | | A | Y | | | Q | | W | S | T | +++ |
| 23 | R | S | S | | G | Q | R | Y | S | | | R | A | F | | | Q | | | S | T | +++ |
| 24 | | S | S | | | Q | R | Y | S | | | R | | Y | | | Q | | | S | T | +++ |
| 25 | R | S | S | | G | Q | R | Y | S | | | | A | Y | | | Q | | Y | S | T | +++ |
| 26 | | S | S | | G | Q | K | Y | S | | | | | F | | | Q | | Y | S | T | +++ |
| 27 | | S | S | | G | Q | R | Y | S | | | | A | Y | | | Q | | Y | S | | +++ |
| 28 | | S | S | | G | Q | R | Y | S | | | | | Y | | | Q | | Y | S | | +++ |
| 29 | R | S | S | | | Q | R | Y | S | | | | A | Y | | | Q | W | W | S | T | +++ |
| 30 | | S | S | | | Q | R | Y | S | | | R | | F | | | Q | | | S | | +++ |
| 31 | | S | S | | G | Q | R | Y | S | | | | A | F | | | Q | W | | S | T | +++ |
| 32 | R | S | S | | G | Q | R | Y | S | | | | A | Y | | R | Q | | | S | | +++ |
| 33 | R | S | S | | G | Q | R | Y | S | | | R | A | F | | | Q | W | Y | S | | +++ |
| 34 | R | S | S | | G | Q | R | Y | S | | T | | | Y | | R | Q | | S | T | | +++ |
| 35 | | S | S | | | | | Y | S | | | | | | | | Q | | | S | | ++* |
| 36 | | S | S | | | | | Y | S | | | | | Y | | | Q | | | S | | +++ |
| 37 | | S | S | | | | | Y | S | | | | | Y | | | Q | | W | S | | +++ |
| 38 | | | | | | | | | | | | | | | | | Q | | | S | | +/− |
| 39 | | S | S | | | | | Y | S | | | | | | | | | | | | | ++ |
| 40 | | | S | | | | | Y | S | | | | | | | | Q | | | S | | +/− |

*reference

Provided in table 11 are several examples of single and/or double amino acid substitutions introduced into the heavy and/or the light chain of M18-G08 (wt). Performance of the variants was analyzed in quadruples in the ELISA with a competition step and the FXa deinhibition assay (FXa DIA). In the ELISA, averages were calculated and overall performance of variants was evaluated by comparing the variant to wt ratio from 1-3 independent experiments. Variants with an average ratio above wt plus 2×SD (standard deviation of the ratio) were considered as improved and are marked with “++”, whereas variants with a ratio below wt minus 2×SD were considered as reduced in their binding affinity and are marked with “−”. All variants with a performance in between both thresholds are marked with “+/−”. Variants with average fluorescence counts below the negative control (non-expressing cells) plus 3×SD were considered as non-binding and marked with “−−”. In the FXa deinhibition assay averages were calculated and overall performance of variants was evaluated by comparing the variant/wt ratio from 2-3 independent experiments. Variants with an average ratio above wt plus 2×SD were considered as improved and are marked with “++”, whereas variants with a ratio below wt minus 2×SD were considered as either reduced in their binding affinity or non-binding and are marked with “−−”. All variants with a performance between both thresholds are marked with “+/−”. Variants not analyzed are marked with “nd” (not determined). CDRs were defined according to Kabat.

TABLE 11
Analysis of single and double amino acid substitutions
within M18-G08

| variable domain | M18-G08 | | |
|---|---|---|---|
| location | mutation | ELISA | FXa-DIA |
| FR1 | HC_G9S | ++ | ++ |
| FR1 | HC_G26A | +/− | +/− |
| FR1 | HC_T28D | +/− | +/− |
| FR1 | HC_S30E | +/− | +/− |
| FR1 | HC_S30G | +/− | +/− |
| FR1 | HC_S30N | +/− | +/− |
| FR1 | HC_S30P | +/− | +/− |
| CDR H1 | HC_N31D | +/− | +/− |
| CDR H1 | HC_N31H | +/− | +/− |
| CDR H1 | HC_N31S | +/− | nd |
| CDR H1 | HC_A32E | +/− | +/− |
| CDR H1 | HC_A32F | +/− | +/− |
| CDR H1 | HC_A32H | +/− | ++ |
| CDR H1 | HC_A32S | +/− | +/− |
| CDR H1 | HC_A32Y | ++ | ++ |
| CDR H1 | HC_M34I | − | Nd |
| CDR H1 | HC_S35A | +/− | +/− |
| CDR H1 | HC_S35N | − | Nd |
| FR2 | HC_S49A | +/− | +/− |
| FR2 | HC_S49G | +/− | +/− |
| CDR H2 | HC_I51V | +/− | +/− |
| CDR H2 | HC_S52D | +/− | +/− |
| CDR H2 | HC_S52G | +/− | +/− |
| CDR H2 | HC_S53I | +/− | +/− |
| CDR H2 | HC_S53T | +/− | +/− |
| CDR H2 | HC_S54D | +/− | +/− |
| CDR H2 | HC_S54E | ++ | +/− |
| CDR H2 | HC_S55D | +/− | +/− |
| CDR H2 | HC_G56S | +/− | Nd |
| CDR H2 | HC_I58A | +/− | +/− |
| CDR H2 | HC_I58R | +/− | ++ |
| CDR H2 | HC_I58S | +/− | +/− |
| CDR H2 | HC_I58T | +/− | +/− |
| CDR H2 | HC_Y59F | +/− | +/− |
| CDR H2 | HC_L64V | +/− | Nd |
| FR3 | HC_A97M | − | +/− |
| FR3 | HC_R98M | +/− | +/− |
| FR3 | HC_R98S | − | ++ |
| FR3 | HC_R98V | +/− | +/− |
| CDR H3 | HC_W100E | +/− | +/− |
| CDR H3 | HC_W100M | +/− | +/− |
| CDR H3 | HC_R101E | +/− | +/− |
| CDR H3 | HC_N102D | − | Nd |
| CDR H3 | HC_H103A | ++ | ++ |
| CDR H3 | HC_H103C | +/− | +/− |
| CDR H3 | HC_H103N | +/− | ++ |
| CDR H3 | HC_H103S | ++ | ++ |
| CDR H3 | HC_H103T | ++ | +/− |
| CDR H3 | HC_H103Y | +/− | +/− |
| CDR H3 | HC_L104F | − | Nd |
| CDR H3 | HC_D105K | +/− | +/− |
| CDR H3 | HC_D105S | +/− | +/− |
| CDR H3 | HC_Y106D | +/− | +/− |
| CDR H3 | HC_Y106V | +/− | ++ |
| FR4 | HC_W107I | +/− | +/− |
| FR4 | HC_W107V | +/− | +/− |
| FR4 | HC_T116S | +/− | Nd |
| FR1 | LC_Q1E | +/− | Nd |
| FR1 | LC_Q6H | +/− | +/− |
| CDR L1 | LC_S23C | − | +/− |
| CDR L1 | LC_G24L | − | +/− |
| CDR L1 | LC_S25G | +/− | +/− |
| CDR L1 | LC_S26G | +/− | +/− |
| CDR L1 | LC_S26K | +/− | +/− |
| CDR L1 | LC_S26R | +/− | +/− |
| CDR L1 | LC_S26V | − | +/− |
| CDR L1 | LC_D28N | +/− | Nd |
| CDR L1 | LC_S31W | +/− | +/− |
| CDR L1 | LC_T33F | +/− | +/− |
| CDR L1 | LC_T33K | ++ | ++ |
| FR2 | LC_Q38K | +/− | +/− |
| FR2 | LC_L47I | +/− | +/− |
| FR2 | LC_L47K | +/− | ++ |
| FR2 | LC_L47S | +/− | +/− |
| FR2 | LC_L47V | +/− | +/− |
| FR2 | LC_I49G | +/− | +/− |
| FR2 | LC_I49L | +/− | +/− |
| FR2 | LC_Y50W | +/− | +/− |
| CDR L2 | LC_D51S | +/− | ++ |
| CDR L2 | LC_Q54V | +/− | +/− |
| CDR L2 | LC_R55A | +/− | +/− |
| CDR L2 | LC_R55G | +/− | ++ |
| CDR L2 | LC_P56K | +/− | +/− |
| CDR L2 | LC_P56R | +/− | +/− |
| CDR L2 | LC_S57R | +/− | +/− |
| FR3 | LC_V59F | +/− | +/− |
| FR3 | LC_S77T | +/− | Nd |
| FR3 | LC_R80Q | +/− | Nd |
| FR3 | LC_S81A | +/− | Nd |
| CDR L3 | LC_Q90E | − | +/− |
| CDR L3 | LC_Q90V | − | +/− |
| CDR L3 | LC_Q90W | − | +/− |
| CDR L3 | LC_S91N | − | +/− |
| CDR L3 | LC_D93S | +/− | +/− |
| CDR L3 | LC_S95C | − | +/− |
| CDR L3 | LC_S95E | +/− | +/− |
| CDR L3 | LC_S95T | +/− | +/− |
| CDR L3 | LC_L96G | +/− | +/− |
| CDR L3 | LC_S97G | +/− | +/− |
| CDR L3 | LC_S97V | +/− | +/− |
| CDR L3 | LC_G98E | +/− | +/− |
| CDR L3 | LC_W99V | − | Nd |
| HC_FR1_LC_CDR L1 | HC_G9V_LC_D51S | +/− | ++ |
| HC_FR1_LC_CDR L1 | HC_G10C_LC_Q54R | ++ | ++ |
| HC_FR1_HC_CDR H2 | HC_L11M_HC_S54D | ++ | +/− |
| HC_FR1_HC_CDR H2 | HC_Q13K_HC_S53V | +/− | +/− |
| HC_FR1_LC_CDR L1 | HC_S21T_LC_S25A | +/− | +/− |
| HC_FR2_LC_CDR L3 | HC_A40T_LC_L96S | +/− | +/− |
| HC_CDR H2_LC_FR4 | HC_S53T_HC_L112P | +/− | +/− |
| HC_CDR H2_LC_FR1 | HC_Q54W_LC_R17H | +/− | +/− |

Provided in Table 12 are some examples of combined amino acid substitutions within M18-G08 antibodies. While not every combination is provided in Table 12, it is contemplated that the M18-G08 anti-rivaroxaban antibody may comprise any combination of modifications provided. Variant performance was analyzed in quadruples in the ELISA with a competition step. Averages were calculated, average background signals determined on a streptavidin coated plate without compound from Example 1K were subtracted if the compound from Example 1K concentration used for coating was below 10 nM and signals were normalized to the respective average expression level. Overall performance of variants was evaluated by comparing the variant to reference ratio from 2-3 independent experiments using 10-fold improved reference variants as compared to wt. For variants with no further indication reference 1 (marked with “*”) was used, for variants marked with # reference 2 (marked with “**”) was used. Variants with an average ratio above reference plus 2×SD are marked with “+++”, whereas variants with a ratio <0.1 of the respective reference are marked with “+/−” with none of the variants fulfilling this criteria. All variants with a performance in between both thresholds are marked with “++”. CDRs were defined according to Kabat.

TABLE 12
Example of multiple amino acid substitutions within M18-G08

| M18- | HC | | | | | | | | | | | LC | | | | | | | | | | | | | | | |
|---|---|---|---|---|---|---|---|---|---|---|---|---|---|---|---|---|---|---|---|---|---|---|---|---|---|---|---|
| G08-X; | FR1 | | CDR H1 | | CDR H2 | | | | CDR H3 | | FR4 | FR1 | CDR L1 | | FR2 | | | CDR L2 | | | FR3 | | | | CDR L3 | | |
| X = | G8 | P14 | N31 | A32 | S53 | G56 | S63 | L64 | H103 | D105 | T116 | A10 | D28 | T33 | A44 | L47 | Y50 | D51 | R55 | S57 | G58 | S77 | R80 | S81 | D93 | S97 | ELISA |
| 1 | | | D | Y | T | S | | V | Y | | S | | N | K | | S | W | S | | | | T | Q | A | | | +++ |
| 2 | | | D | Y | T | S | | V | Y | | S | | N | K | | V | W | S | | | | T | Q | A | | V | +++ |
| 3 | | | D | Y | | S | | V | Y | S | S | | N | K | | V | W | S | | | | T | Q | A | | | +++ |
| 4 | | | D | Y | T | S | | V | Y | S | S | | N | K | | V | W | S | | | | T | Q | A | | | +++ |
| 5 | | | D | Y | | S | | V | Y | S | S | | N | K | | S | | S | | | | T | Q | A | S | | +++ |
| 6 | | | D | Y | T | S | | V | Y | S | S | | N | K | | S | | S | | | | T | Q | A | S | V | +++ |
| 7 | | | D | Y | | S | | V | Y | S | S | | N | K | | S | W | S | | | | T | Q | A | | V | +++ |
| 8 | | | D | Y | T | S | | V | Y | S | S | | N | K | | S | W | S | | | | T | Q | A | S | | +++ |
| 9 | | | D | Y | T | S | | V | | | S | | N | K | S | I | W | S | | | | T | Q | A | S | V | +++ |
| 10 | | | D | Y | T | S | | V | S | | S | | N | K | | | W | S | | | | T | Q | A | | V | +++ |
| 11 | | | D | Y | T | S | | V | S | | S | | N | K | | | W | S | | | | T | Q | A | | | +++ |
| 12 | | | D | Y | T | S | | V | Y | S | S | | N | | | V | W | S | | | | T | Q | A | S | | +++ |
| 13 | | | D | Y | | S | | V | Y | S | S | | N | | | S | | S | | | | T | Q | A | S | | +++ |
| 14 | | | D | Y | | S | | V | Y | S | S | | N | K | | V | W | S | H | | | T | Q | A | | V | +++ |
| 15 | | | D | Y | | S | | V | S | S | S | | N | | | S | | S | | | | T | Q | A | S | | ++ |
| 16 | | | D | Y | T | S | G | V | Y | S | S | | N | K | | I | W | S | | | | T | Q | A | | | +++ |
| 17 | | | D | Y | | S | | V | Y | S | S | | N | K | | V | | S | | | | T | Q | A | S | V | +++ |
| 18 | | | D | Y | T | S | | V | Y | S | S | | N | K | | V | | S | | | | T | Q | A | | V | +++ |
| 19 | | | D | Y | | S | | V | Y | S | S | | N | K | | S | W | S | | N | | T | Q | A | | | +++ |
| 20 | | | D | Y | | S | | V | Y | | S | | N | K | | I | W | S | | | | T | Q | A | S | V | ++ |
| 21 | | | D | Y | T | S | | V | Y | S | S | | N | K | | S | | S | | | | T | Q | A | S | | +++ |
| 22 | | | D | Y | T | S | | V | Y | | S | | N | | | S | W | S | | | | T | Q | A | S | | +++ |
| 23 | | | D | Y | T | S | | V | Y | | S | | N | | | S | W | S | | | | T | Q | A | S | | +++ |
| 24 | | | D | Y | | S | | V | Y | S | S | | N | K | | S | | S | | | | T | Q | A | | V | +++ |
| 25 | | | D | Y | T | S | | V | Y | S | S | | N | K | | I | W | S | | | | T | Q | A | S | | +++ |
| 26 | | | D | Y | | S | | V | Y | S | S | | N | K | | V | W | S | | | | T | Q | A | S | V | +++ |
| 27 | | | D | Y | T | S | | V | Y | | S | | N | K | | S | W | S | | | | T | Q | A | S | V | +++ |
| 28 | | | D | Y | | S | | V | Y | S | S | T | N | | | V | | S | | | | T | Q | A | S | | +++ |
| 29 | | | D | Y | T | S | | V | A | | S | | N | K | | | | S | | | | T | Q | A | S | | +++ |
| 30 | | | D | Y | T | S | | V | A | | S | | N | | | | W | S | | | | T | Q | A | S | V | ++ |
| 31 | | | D | Y | | S | | V | Y | S | S | | N | K | | I | W | S | | | | T | Q | A | S | V | +++ |
| 32 | | | D | Y | T | S | | V | Y | S | S | | N | K | | V | W | S | | | S | T | Q | A | S | | +++ |
| 33 | | T | D | Y | T | S | | V | | S | S | | N | K | | V | W | S | | | | T | Q | A | S | V | +++ |
| 34 | | | D | Y | T | S | | V | Y | S | S | | N | K | | S | W | S | | | | T | Q | A | | V | +++ |
| 35 | | | D | Y | T | S | | V | Y | S | S | | N | | | I | W | S | | | | T | Q | A | S | | +++ |
| 36 | | | D | Y | T | S | | V | Y | | S | | N | K | | S | W | S | | | | T | Q | A | S | | ++ |
| 37 | | | D | Y | T | S | | V | Y | S | S | | N | K | | V | | S | | N | | T | Q | A | S | | +++ |
| 38 | | | D | Y | T | S | | V | Y | S | S | | N | K | | I | W | S | | | | T | Q | A | | | +++ |
| 39 | | | D | Y | T | S | | V | Y | S | S | | N | K | | V | W | S | | | | T | Q | A | | V | +++ |
| 40 | | | D | Y | | S | | V | Y | S | S | | N | K | | S | W | S | | | | T | Q | A | S | V | +++ |
| 41 | | | D | Y | T | S | | V | A | S | S | | N | | | | W | S | | | | T | Q | A | | | +++ |
| 42 | | | D | Y | T | S | | V | Y | S | S | | N | | | S | | S | | | | T | Q | A | S | V | +++ |
| 43 | | | D | Y | | S | | V | Y | | S | | N | | | V | W | S | | | | T | Q | A | S | V | +++ |
| 44 | | | D | Y | T | S | | V | A | S | S | | N | | | I | W | S | | | | T | Q | A | S | V | +++ |
| 45 | | | D | Y | T | S | | V | Y | S | S | | N | K | | S | | S | | | | T | Q | A | | | +++ |
| 46 | | | D | Y | T | S | | V | S | S | S | | N | K | | S | | S | | | | T | Q | A | S | V | +++ |
| 47 | | | D | Y | T | S | | V | Y | S | S | | N | K | | I | W | S | | | | T | Q | A | S | V | +++ |
| 48 | | | D | Y | T | S | | V | Y | | S | | N | K | | V | | S | | | | T | Q | A | S | V | +++ |
| 49 | | | D | Y | T | S | | V | Y | S | S | | N | K | | S | W | S | | | | T | Q | A | | | +++ |
| 50 | | | D | Y | T | S | | V | Y | | S | | N | K | | I | W | S | | | | T | Q | A | S | V | +++ |
| 51 | | | D | Y | T | S | | V | Y | S | S | | N | | | I | W | S | | | | T | Q | A | S | | +++ |
| 52 | | | S | Y | | S | | V | S | | S | | N | | | | | S | | | | T | Q | A | | V | ++ |
| 53 | | | S | Y | | S | | V | S | | S | | N | | | | | S | | | | T | Q | A | | | ++* |
| 54 | | | S | Y | | S | | V | Y | | S | | N | | | | | S | | | | T | Q | A | | V | ++ |
| 55 | | | | Y | | | | V | Y | | S | | N | | | | | S | | | | T | Q | A | | | ++ |
| 56 | | | | Y | | | | V | Y | | S | | N | | | | | S | | | | T | Q | A | | V | ++ |
| 57 | | | | Y | | | | V | S | | S | | N | | | | | S | | | | T | Q | A | | V | ++ |
| 58 | | | | Y | | | | V | S | | S | | N | | | | | S | | | | T | Q | A | | | +++ |
| G | | | S | Y | | S | | V | Y | | S | | N | | | | | S | | | | T | Q | A | | | ++** |
| 62 | | | | Y | | | | V | Y | | S | | | | | | | | | | | T | Q | A | | | ++# |
| 63 | | | S | Y | | S | | V | Y | | S | | | | | | | | | | | T | Q | A | | | ++# |
| 64 | | | S | Y | | S | | V | Y | | S | | | | | | | | | | | | | | | | ++# |
| 65 | | | | | | | | | | | | | N | | | | | S | | | | T | Q | A | | | ++# |

*reference 1;
**reference 2;
#variants analysed using reference 2

### Example 15

### Purification of his-Tagged Fab Fragments

Cells were harvested by centrifugation at 9000 rpm for 30 min at 4° C. and stored at −20° C. The antibody fragments were purified from the supernatant using a two-step purification procedure. First, the supernatant was re-buffered against buffer A (50 mM NaH2PO₄, 300 mM NaCl, 10 mM imidazole pH 8.0) and concentrated and 100 ml were loaded on a 5 ml Ni-NTA superflow column (Qiagen, 1018142). After loading the column was washed first with 20 column volumes of buffer A followed by 15 column volumes of 4.3% buffer B (50 mM NaH2PO₄, 300 mM NaCl, 250 mM imidazole pH 8.0) and eluted with 15 volumes of buffer B. Fractions were combined and the buffer was exchanged to PBS using PD-10 columns according to the manufacturer's protocol (GE Healthcare, 17-0851-01). In a second purification step the Ni-NTA purified antibodies were incubated with the capture select lambda affinity matrix (BAC 0849.010). After incubation at 4° C. over night the matrix was loaded into a column, washed with 5 volumes of PBS, 5 volumes of 500 mM arginine 100 mM NaH2PO₄, 100 mM NaCl pH6.0 and again 5 volumes of PBS. Antibodies were eluted with 6 volumes of 100 mM glycine pH3.0 followed by 3 volumes of 100 mM glycine pH 2.0 into 1/15 of total volume of 1M HEPES pH 7.5 to neutralize the eluates. Finally, eluates were dialyzed against PBS over night at 4° C. Purified antibodies were analyzed by SDS-PAGE and mass spectrometry.

### Example 16

### Production of Non-Tagged Fab Fragments in E. Coli and in HEK293

### Cloning of Expression-Constructs:

Routine cloning tasks were carried out according Sambrook et al. (Molecular Cloning Cold Spring Harbor, 1989). For the isolation of plasmid DNA from E. coli (minipreps) Qiagen-tips (Qiagen) were used. The host organism employed for transformation was E. coli strain DHSalpha (invitrogen). The extraction of DNA fragments from agarose gels was carried out with the aid of Qiagen gel extraction kit according to the manufacturers protocol (Qiagen). Oligonucleotides for PCR and sequencing reactions were purchased from metabion, synthetic genes (optimized for S. cerevisiae coden-usage) from Geneart. For PCR experiments the KOD Polymerase from Merck was used according to the manufacturer's protocol. All vector constructs were confirmed by external sequencing (eurofins).

E. Coli Expression Vectors pMC11 and pMC14:

Recloning of both, M18-G08-DKTHT and M18-G08-G-DKTHT from pET28a into pUC based E. coli expression vectors (pMC14 and pMC11, respectively) under the control of the pLAC promoter was done by amplifying light chain (LC) and heavy chain (HC) sequences separately, followed by the use of unique restriction sites of the pUC based vector.

Additional oligonucleotides 5′ and 3′ to the coding sequence include restriction enzyme recognition sites, which were used for in-frame subcloning of LC and HC into the pUC based E. coli expression vector:
Amplification of LC sequences was performed with a forward primer carrying the NheI-restriction site binding to the pelB leader and a reverse primer pairing to the 3′ end of the LC sequence. Restriction was done with NheI/XhoI. Subsequent ligation was performed into NheI/XhoI digested pUC based vector. Transformation of ligated DNA was done in E. coli DHSalpha (invitrogen).

Amplification of HC sequences was performed with a forward primer carrying the NcoI-restriction site binding to the pelB leader and a reverse primer pairing to the 3′ end of the LC sequence and carrying additional nucleotides encoding for the terminal 5 amino acid DKTHT followed by a SacII-restriction site. Restriction was done with NcoI/SacII. Subsequent ligation was performed into NcoI/SacII digested pUC based vector. Transformation of ligated DNA was done in E. coli DH5alpha (invitrogen).

The protein sequence of the gene coding for the LC of M18-G08-DKTHT is as following:

QSVLTQPPSASGTPGQRVTISCSGSSSDIGSNTVNWYQQLPGTAPKLLIY
DNNQRPSGVPDRFSGSKSGTSASLAISGLRSEDEADYYCQSYDSSLSGWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The protein sequence of the gene coding for the HC of M18-G08-DKTHT is as following:

EVQLLESGGGLVQPGGSLRLSCAASGFTFSNAWMSWVRQAPGKGLEWVSS
ISSSSGYIYYADSLKGRFTISRDNSKNTLYLQMNSLRAEDTAVYYCARVW
RNHLDYWGQGTLVTVTSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHT

The protein sequence of the gene coding for the LC of M18-G08-G-DKTHT is as following:

QSVLTQPPSASGTPGQRVTISCSGSSSNIGSNTVNWYQQLPGTAPKLLIY
SNNQRPSGVPDRFSGSKSGTSASLAITGLQAEDEADYYCQSYDSSLSGWV
FGGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQVT
HEGSTVEKTVAPTECS

The protein sequence of the gene coding for the HC of M18-G08-G-DKTHT is as following:

EVQLLESGGGLVQPGGSLRLSCAASGFTFSSYWMSWVRQAPGKGLEWVSS
ISSSSSYIYYADSVKGRFTISRDNSKNTLYLQMNSLRAEDTAVYYCARVW
RNYLDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHT

Mammalian Expression Vectors pMC19 And pMC32:
The coding sequence regarding to plasmids pMC19 (encoding for M18-G08-G-DKTHT) and pMC32 (encoding for M18-G08-DKTHT) were purchased as synthetic genes from Geneart (optimized for mammalien codon-usage). Additional oligonucleotides 5′ and 3′ to the coding sequence include restriction enzyme recognition sites, which were used for in-frame subcloning of the respective sequences into a single standard mammalian expression vector under the control of the pCMV5 promoter.

### Fab Expression in HEK293 6E Cells

Some Fab fragements were produced by mammalian cell culture using transiently transfected HEK293 6E cells. Heavy and light chain were cloned both into a single vector system under control of the CMV5 promotor as described above. Expression scale was 10 L in 20 L wave bags (Cultibag, Sartorius) utilizing F17 medium (Invitrogen, order no.: 05-0092DK) supplemented 24 h after transfection with 0.5% trypone TN1 (Organotechnie, order no.: 19553) and 1 FCS ultra low IgG (Invitrogen, order no.: 16250). Cells were cultured for 6 days at 5% CO2, 37° C., 18 rocks/min with an angle of 8°. Expression level was approximately 120 mg/L. Cells were harvested by centrifugation (Sorvall RC12BP, 30 min, 4° C., 4000 rpm). Cells were discarded. The supernatant containing the Fab fragments was filtered through a 0.2 μm sterilfilter (Sartorius Sartopore 2 XLG, order no.: 5445307G8-1-00).
Fab-Expression in E. coli BL21:
Fab fragments can also be expressed in E. coli systems based on expression constructs described above. 200 ml 2×YT medium (Difco 2xYT Medium: Becton Dickinson (BD), order no.: 244020) supplemented with 10 g/L glucose-monohydrat (Sigma, order no.: G5767) and 100 mg/L carbenicilline (AppliChem, order no.: A1491,0010) were inoculated with 1000 μl of a cryoculture of E. coli BL21 transformed with e.g. pMC11 and incubated at 250 rpm at 37° C. for 16 h. This seed train was used for inoculation of 20 L 2xYT medium supplemented with 1 g/L glucose-monohydrate, 100 mg/L carbenicilline and 0.1 ml/L polyglycol P2000 (BASF). The production culture was incubated in a 50 L wave bag at a Sartorius Cultibag at 30° C., 35 rocks/min, an angle of 9° and an aeration rate of 0.52 L air/min. At an OD600 of 0.5-0.6 expression of the Fab fragment was induced by 0.75 mM IPTG. After further 20 h incubation, the culture was harvest by centrifugation (Sorvall RC12BP, 1 h, 4° C., 4000 rpm). The biomass was frozen at −20° C., the supernatant was filtered through a 0.2 μm sterilfilter (Sartorius Sartopore 2 XLG, order no.: 5445307G8-1-00) and concentrated with a Millipore Pro Flux M12 cross flow filtration using a Millipore Pellicon-Mini-Holder with 2 Sartorius Slice cassettes Hydrosart 10 k. The used parameters were: Inlet pressure: 2 bar; outlet pressure 1.5 bar, differential pressure: 0.5 bar, yielding at the beginning 100 ml filtrate in 50 seconds.
100 g E. coli cell pellet were resuspended in 320 ml of 30 mM Tris/HCl pH 8.0+200 g/L sucrose+6 tablets EDTA-free Protease Inhibitor (Roche)+25 U/ml Benzonase (Sigma E1014)+1 mg/ml lysozyme (Sigma) and incubated at 4° C. for 2 h. cells were disrupted using a TS cell disruption system (Constant Systems, Ltd.) with 40 KPSI head installed (working pressure 36,1KPSI) 2 cycles, Cell temperature 10° C.; feed solution and flow-through were kept at 4° C. Cell debris was removed by centrifugation at 4° C. for 35 mM at 35,000×g. The cleared supernatant (=crude extract) was sterile filtered and stored at −20° C. for further processing.

### Purification of Untagged Fabs.

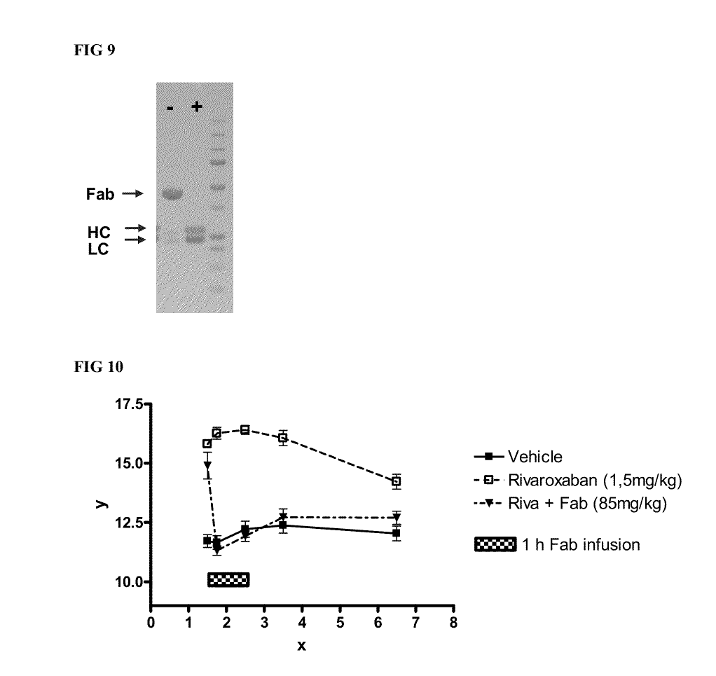

The Fab fragments of the invention were purified from sterile filtered HEK293 6E or E. coli supernatants, or from sterile filtered E. coli crude extracts (generated as described above) using a 2-step purification method. As capture step a “lambda select” affinity column (BAC) was used. For 10 L of cell supernatant or E. coli culture 40 ml of lambda select resin was used. All further purification steps were conducted at 20° C. the flow rate used was 6 ml/min for all subsequent steps. The column was equilibrated in 5 column volumes (=CV) of PBS pH 7.4. The sample was pH adjusted to pH 7.4 with an 1M NaOH solution and applied to the column Followed by a wash with 10 CV of PBS pH 7.4. Fab fragments were eluted with 3CV of 50 mM Na-acetate, 500 mM NaCl pH3.5. The elution pool was pH adjusted to pH 7.0 with 2.5 M Tris, sterile filtered and concentrated to 17.8 mg using a ultrafiltration device (Amicon Ulta 10 kDa, Millipore UFC901008). The sample was applied to a 35/500 Superdex 75 size exclusion column (GE Healthcare), equilibrated in PBS pH 7.4. The column was eluted at a flow rate of 3 ml/ml for 2 CV and fractions of 6 ml were collected. Fractions containing Fab-fragments were pooled and analyzed via SDS PAGE (4-12% NuPage, Invitrogen). As shown in FIG. 9 the preparation of M18-G08-G-DKTHT is >98% pure with both light cahin=LC and heavy chain ═HC present in equal amounts.

### Example 17

### Prothrombin Time (PT) Ex Vivo (Rat PD/PK)

Rivaroxaban was administered to male Wistar rats (Charles River) by oral gavage at a dose of 1.5 mg/kg dissolved in EtOH-PEG-water (10-50-40%, 5 ml/kg). Isoflurane anesthesia was induced at ˜75 minutes after oral dosing for implantation of a venous (V. jugularis) catheter for Fab-antidote infusion and of an arterial (A. carotis) catheter for blood sampling. At 90 minutes post oral rivaroxaban dosing, infusion of Fab M18-G08-G-DKTHT was started at a dose of 85 mg/kg within one hour (in PBS, administration volume 15 ml/kg/h). Prothrombin times and plasma concentrations were determined as described in Example 10. Normalization of Rivaroxaban-Induced PT Prolongation is Shown in FIG. 10. A time course of plasma concentrations of unbound rivaroxaban for this experimental setting is described in Example 18.

### Example 18

### Reduction of the Unbound Rivaroxaban Concentrations by Fab Antidote in Rats

In order to investigate the in vivo effect of the Fab antidote on the pharmacokinetics of rivaroxban unbound concentration of rivaroxaban in rats following co-administration of Fab M18-G08-G-DKTHT were determined in conscious and anesthetized rats (following the protocol describe in Example 17). As only the unbound concentration of drug will drive the pharmacological effect, unbound concentrations are a good predictor for the pharmacological effect. For the determination of the unbound concentration plasma samples were diluted with DPBS (1/1 v/v) and than added to an ultrafiltration device containing a membrane with an exclusion size of 30,000 Da. Samples were centrifuged for 2 min at 1200 g. 25 μL of the utrafiltrate was diluted with DPBS (1/1 v/v) and than added to an ultrafiltration device containing a membrane with an exclusion size of 30000 Da. Samples were centrifuged for 2 min at 1200 g. 25 μL of the utrafiltrate was diluted with 25 μL DPBS and spiked with 150 μL of a solution of ammonium acetate/acetonitril (1/1 v/v) pH 6.8 containing the internal standard. Plasma samples were spiked with 300 μL acetonitril and centrifuged at 2000 g for 10 min at 4° C. All samples were analyzed by LC-MS/MS using an API 4000 (AB Sciex). The fu values were calculated according to the relation fu (%)=concentration filtrate/concentration plasma sample*100 and were corrected for unspecific binding to the ultrafiltration device and plasma dilution as described before (Schuhmacher J. et al., J Pharm Sci. 2004; 93(4):816-30).

FIG. 11 depicts a concentration/time profile of unbound rivaroxaban in rat plasma following oral administration of 1.5 mg/kg rivaroxaban and infusion of 85 mg/kg Fab M18-G08-G-DKTHT over 1 h starting 1.5 h after administration of rivaroxaban. A rapid reduction of the unbound plasma concentrations of rivaroxaban following infusion of the Fab is shown. For some samples the unbound concentration of rivaroxaban could not be determined because they were below the lower limit of quantification (LLOQ).

### Example 19

### Bleeding Time in Rats

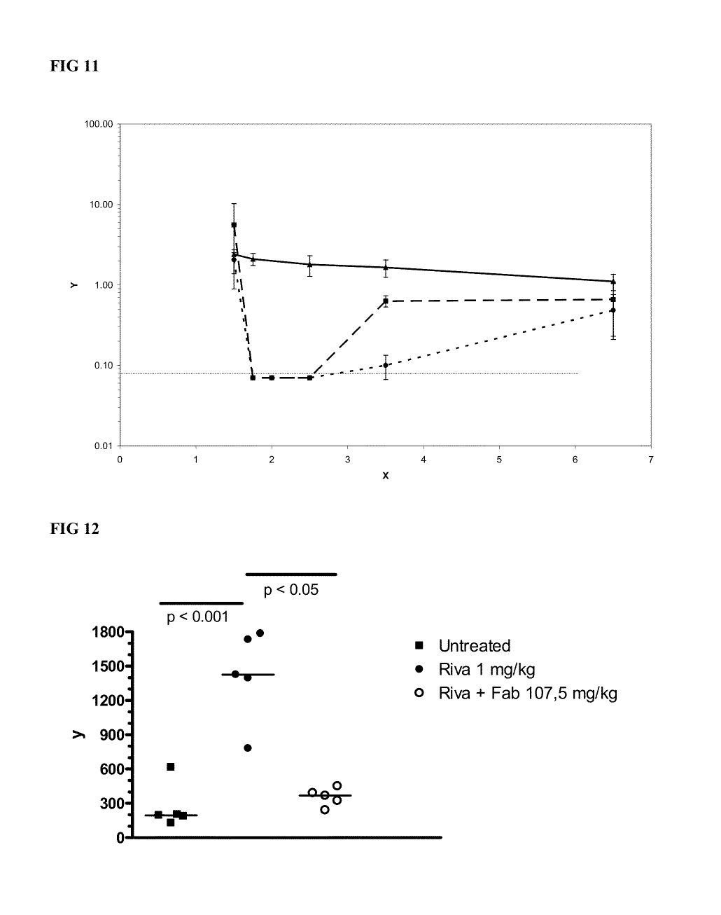

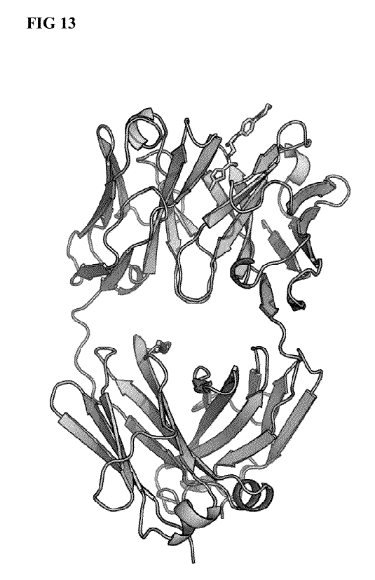

The potential of the Fab M18-G08-G-DKTHT to interfere with bleedings prolonged by rivaroxaban was studied in the rat tail transsection model Animals (male Wistar rats, Charles River) were anesthetized with inactin (180 mg/kg i.p.) for implantation of venous (V. jugularis) and arterial (A. carotis) catheters for drug administration and blood sampling. Five minutes after an iv. bolus administration of 1 mg/kg rivaroxaban (in EtOH-PEG-water, 10-50-40%, 3 ml/kg)., bleeding was initiated by transsecting the tail at a distance of 2-3 mm proximal to the tip. The bleeding tail was immersed into 0.9% 37° C. saline for observation and recording of the bleeding event. One minute after initiation of the bleeding, a 10 minute infusion of Fab M18-G08-G-DKTHT at a dose 107.5 mg/kg (in PBS, 1.33 ml/kg/min) was started. Bleeding was recorded for 30 minutes and scored (0=no bleeding, 1=minimal, 2=mild, 3=moderate, 4=maximal bleeding) by an observer blinded to the treatment. The scores were obtained in 30-sec. intervals and cumulative bleeding time was calculated with the maximal value of 1800 sec achieved in case of a 30 min score 4 bleeding. Median bleeding times of 5 animals per group were 195, 1425 and 368 seconds in untreated, rivaroxaban and rivaroxaban plus Fab-antidote groups, respectively (FIG. 12).

### Example 20

### Crystallization and X-Ray Structure Determination of Fab M18-G08-G-DKTHT-Rivaroxaban Complex

### Crystallization

The protein comprising Fab M18-G08-G-DKTHT was concentrated to 30 mg/ml in 10 mM Hepes pH at 7.0. Prior crystallization the protein solution was mixed with a three fold molar excess of rivaroxaban dissolved in 50 mM DMSO and incubated for one hour on ice. Co-crystals of the protein construct comprising Fab M18-G08-G-DKTHT and rivaroxaban were grown at 20° C. using the sitting-drop method and crystallized by mixing equal volumes of protein solution and well solution (100 mM TRIS pH 7.0, 20% PEG4000, 2M NaCl) as precipitant. Crystals appeared after one day and grew to its final size after 14 days.

### Data Collection and Processing

Crystal was flash-frozen in liquid nitrogen without use of cryo-buffer. Data of crystal was collected at beamline BL14.1, BESSY synchrotron (Berlin) on a MAR CCD detector. Data was indexed and integrated with XDS (Kabsch, W. (2010) Acta Cryst. D66, 125-132), prepared for scaling with POINTLESS, and scaled with SCALA (P. R. Evans, (2005) Acta Cryst. D62, 72-82). The crystal diffracted up to 2.25 Å and possesses cubic space group P23 with cell constant a=120.4, and one Fab M18-G08-G-DKTHT-Rivaroxaban complex in the asymmetric unit.

### Structure Determination and Refinement

The co-structure of rivaroxaban and the monoclonal antibody Fab M18-G08-G-DKTHT was solved by molecular replacement using BALBES (F. Long, A. Vagin, P. Young and G. N. Murshudov (2008) Acta Cryst. D64, 125-132), with pdb code 1rzf as search model. Iterative rounds of model building with COOT (P. Emsley et al. (2010) Acta Cryst. D66:486-501) and maximum likelihood refinement using REFMAC5.5 (G. N. Murshudov et al. (1997) Acta Cryst. D53, 240-255) completed the model. Data set and refinement statistics are summarized in table 13.

TABLE 13
Data set and refinement statistics for Fab M18-G08-G-DKTHT-
rivaroxaban complex.

| Wavelength | 0.9184 Å |
|---|---|
| Resolution (highest shell) | 37.44-2.25 (2.31-2.25) Å |
| Reflections (observed/unique) | 402848/27895 |
| Completeness^{a} | 100.0% (100.0%) |
| I/s^{a} | 8.28 (1.59) |
| R_{merge}^{a,b} | 0.07 (0.43) |
| Space group | P23 |
| Unit cell parameters | |
| a | 120.43 Å |
| R_{cryst}^{c} | 0.183 |
| R_{free}^{d} | 0.234 |
| Wilson temperature factor | 39.9 Å² |
| RMSD bond length^{e} | 0.023 Å |
| RMSD bond angles | 2.045° |
| Protein atoms | 3233 |
| Water molecules | 258 |

^{a}The values in parentheses are for the high resolution shell.
^{b}R_{merge} = Σhkl |Iₕₖₗ − <Iₕₖₗ>|/Σhkl <Iₕₖₗ> where Iₕₖₗ is the intensity of reflection hkl and <Iₕₖₗ> is the average intensity of multiple observations.
^{c}R_{cryst} = Σ |F_{obs} − F_{calc}|/Σ F_{obs} where F_{obs} and F_{calc} are the observed and calculated structure factor amplitues, respectively.
^{d}5% test set
^{e}RMSD, root mean square deviation from the parameter set for ideal stereochemistry

### Example 21

### X-Ray Structure-Based Epitope Mapping

The complex of Fab M18-G08-G-DKTHT and rivaroxaban (FIG. 13) crystallized as one copy of the complex per asymmetric unit. Residues of Fab M18-G08-G-DKTHT in contact with rivaroxaban (the paratope). Two methods were used to determine the binding epitope of rivaroxaban to Fab M18-G08-G-DKTHT and are listed in table 14 a and b.
Method 1: Buried surface was analysed with the CCP4 program AREAIMOL (P. J. Briggs (2000) CCP4 Newsletter No. 38) and residues showing a total area difference when calculated with bound and without bound rivaroxaban (table 14a). Method 2: For the calculation, hydrogen atoms were added to all amino acids of Fab M18-G08-G-DKTHT as well at to rivaroxaban. Then residues in a 4A environment of bound rivaroxaban in the crystal structure were calculated using the program Discovery Studio, Version 3.1 (Accelrys Software Inc., 2005-11) (table 14b).
All residues originating from both calculations have been considered to be contacted to rivaroxaban.

TABLE 14a
Residues of Fab M18-G08-G-DKTHT in contact with rivaroxaban
using method 1.

| | | Area |
|---|---|---|
| Chain | Residue | Differences |
| LC | GLN90 | −6.00 |
| LC | TRP99 | −25.30 |
| LC | PHE101 | −1.80 |
| HC | SER 31 | −4.40 |
| HC | TRP33 | −67.30 |
| HC | SER35 | −0.70 |
| HC | TRP47 | −4.60 |
| HC | SER50 | −6.50 |
| HC | SER53 | −6.00 |
| HC | TYR57 | −3.40 |
| HC | VAL99 | −18.70 |
| HC | TRP100 | −0.20 |
| HC | ARG101 | −11.20 |
| HC | ASN102 | −27.20 |
| HC | TYR103 | −0.80 |
| HC | LEU104 | −15.00 |

TABLE 14b
residues of Fab M18-G08-G-DKTHT in contact with rivaroxaban
calculated using method 2. All distances in Å are listed.

| Chain | Residue | Atom1 | Distance [Å] | Atom2 |
|---|---|---|---|---|
| LC | ASN35 | HD21 | 3.21 | H2 |
| LC | ASN35 | HD22 | 3.92 | H2 |
| LC | ASN35 | ND2 | 3.95 | H2 |
| LC | TYR37 | HH | 3.20 | H2 |
| LC | TYR37 | OH | 3.93 | H2 |
| LC | GLN90 | CD | 2.89 | H2 |
| LC | GLN90 | HE21 | 3.73 | H2 |
| LC | GLN90 | HE22 | 2.67 | H2 |
| LC | GLN90 | NE2 | 2.96 | H2 |
| LC | GLN90 | OE1 | 2.54 | H2 |
| LC | GLN90 | HG2 | 3.86 | CL29 |
| LC | GLN90 | HG1 | 3.19 | CL29 |
| LC | GLN90 | CG | 3.82 | CL29 |
| LC | GLN90 | CD | 3.75 | CL29 |
| LC | GLN90 | OE1 | 3.54 | C28 |
| LC | GLN90 | NE2 | 3.60 | C28 |
| LC | GLN90 | HE22 | 3.31 | C28 |
| LC | GLN90 | CD | 3.71 | C28 |
| LC | TRP99 | HB2 | 3.57 | S24 |
| LC | TRP99 | HB1 | 3.91 | S24 |
| LC | TRP99 | CG | 3.57 | S24 |
| LC | TRP99 | CD2 | 3.79 | S24 |
| LC | TRP99 | CD1 | 3.96 | S24 |
| LC | TRP99 | CB | 3.93 | S24 |
| LC | TRP99 | CZ2 | 3.86 | O20 |
| LC | TRP99 | CH2 | 3.94 | O20 |
| LC | TRP99 | CE2 | 3.95 | O20 |
| LC | TRP99 | NE1 | 3.67 | N14 |
| LC | TRP99 | HZ2 | 3.33 | N14 |
| LC | TRP99 | HE1 | 3.56 | N14 |
| LC | TRP99 | CZ2 | 3.45 | N14 |
| LC | TRP99 | CE2 | 3.55 | N14 |
| LC | TRP99 | HZ2 | 3.89 | H8 |
| LC | TRP99 | CE2 | 3.94 | H6 |
| LC | TRP99 | CZ2 | 3.22 | H6 |
| LC | TRP99 | HE1 | 3.49 | H6 |
| LC | TRP99 | HZ2 | 2.30 | H6 |
| LC | TRP99 | CE2 | 3.78 | H5 |
| LC | TRP99 | CH2 | 3.14 | H5 |
| LC | TRP99 | CZ2 | 2.92 | H5 |
| LC | TRP99 | HH2 | 3.12 | H5 |
| LC | TRP99 | HZ2 | 2.72 | H5 |
| LC | TRP99 | CE2 | 3.62 | H3 |
| LC | TRP99 | CZ2 | 3.71 | H3 |
| LC | TRP99 | HE1 | 3.04 | H3 |
| LC | TRP99 | HZ2 | 3.46 | H3 |
| LC | TRP99 | NE1 | 3.37 | H3 |
| LC | TRP99 | CD1 | 3.98 | H2 |
| LC | TRP99 | HD1 | 3.52 | H2 |
| LC | TRP99 | CD1 | 3.82 | H1 |
| LC | TRP99 | HD1 | 3.86 | H1 |
| LC | TRP99 | HE1 | 3.39 | H1 |
| LC | TRP99 | NE1 | 3.59 | H1 |
| LC | TRP99 | HB2 | 3.65 | CL29 |
| LC | TRP99 | HB1 | 3.13 | CL29 |
| LC | TRP99 | CB | 3.85 | CL29 |
| LC | TRP99 | HZ2 | 3.05 | C7 |
| LC | TRP99 | CZ2 | 3.82 | C7 |
| LC | TRP99 | HZ2 | 3.92 | C3 |
| LC | TRP99 | HD1 | 3.41 | C28 |
| LC | TRP99 | HB1 | 3.87 | C28 |
| LC | TRP99 | CG | 3.95 | C28 |
| LC | TRP99 | CD1 | 3.56 | C28 |
| LC | TRP99 | HD1 | 3.79 | C27 |
| LC | TRP99 | HB2 | 3.69 | C27 |
| LC | TRP99 | HB1 | 3.29 | C27 |
| LC | TRP99 | CG | 3.68 | C27 |
| LC | TRP99 | CD1 | 3.77 | C27 |
| LC | TRP99 | CB | 3.77 | C27 |
| LC | TRP99 | NE1 | 3.52 | C25 |
| LC | TRP99 | HE1 | 3.61 | C25 |
| LC | TRP99 | HD1 | 3.61 | C25 |
| LC | TRP99 | CG | 3.98 | C25 |
| LC | TRP99 | CD1 | 3.48 | C25 |
| LC | TRP99 | NE1 | 3.56 | C19 |
| LC | TRP99 | HE1 | 3.87 | C19 |
| LC | TRP99 | CG | 3.76 | C19 |
| LC | TRP99 | CE2 | 3.64 | C19 |
| LC | TRP99 | CD2 | 3.78 | C19 |
| LC | TRP99 | CD1 | 3.65 | C19 |
| LC | TRP99 | NE1 | 3.80 | C16 |
| LC | TRP99 | HZ2 | 3.92 | C16 |
| LC | TRP99 | CZ2 | 3.65 | C16 |
| LC | TRP99 | CE2 | 3.51 | C16 |
| LC | TRP99 | CD2 | 3.94 | C16 |
| LC | TRP99 | HZ2 | 3.15 | C12 |
| LC | TRP99 | CZ2 | 3.51 | C12 |
| LC | PHE101 | HZ | 3.53 | CL29 |
| LC | PHE101 | HE1 | 3.40 | CL29 |
| LC | PHE101 | CZ | 3.83 | CL29 |
| LC | PHE101 | CE1 | 3.75 | CL29 |
| HC | TRP33 | HE3 | 3.20 | O20 |
| HC | TRP33 | HE1 | 3.60 | N15 |
| HC | TRP33 | CZ2 | 3.83 | H9 |
| HC | TRP33 | HZ2 | 3.62 | H9 |
| HC | TRP33 | CD2 | 2.95 | H7 |
| HC | TRP33 | CE2 | 2.88 | H7 |
| HC | TRP33 | CE3 | 2.96 | H7 |
| HC | TRP33 | CG | 3.85 | H7 |
| HC | TRP33 | CH2 | 2.87 | H7 |
| HC | TRP33 | CZ2 | 2.88 | H7 |
| HC | TRP33 | CZ3 | 2.92 | H7 |
| HC | TRP33 | HE3 | 3.62 | H7 |
| HC | TRP33 | HH2 | 3.48 | H7 |
| HC | TRP33 | HZ2 | 3.52 | H7 |
| HC | TRP33 | HZ3 | 3.58 | H7 |
| HC | TRP33 | NE1 | 3.73 | H7 |
| HC | TRP33 | CD2 | 3.86 | H5 |
| HC | TRP33 | CE3 | 2.96 | H5 |
| HC | TRP33 | CH2 | 3.95 | H5 |
| HC | TRP33 | CZ3 | 3.03 | H5 |
| HC | TRP33 | HE3 | 2.81 | H5 |
| HC | TRP33 | HZ3 | 2.95 | H5 |
| HC | TRP33 | CB | 3.63 | H4 |
| HC | TRP33 | CD2 | 3.25 | H4 |
| HC | TRP33 | CE3 | 2.89 | H4 |
| HC | TRP33 | CG | 3.61 | H4 |
| HC | TRP33 | CZ3 | 3.65 | H4 |
| HC | TRP33 | HB1 | 3.20 | H4 |
| HC | TRP33 | HB2 | 3.41 | H4 |
| HC | TRP33 | HE3 | 2.66 | H4 |
| HC | TRP33 | HZ3 | 3.98 | H4 |
| HC | TRP33 | HE1 | 2.59 | H14 |
| HC | TRP33 | NE1 | 3.57 | H14 |
| HC | TRP33 | HE1 | 3.31 | H13 |
| HC | TRP33 | CD1 | 3.78 | H12 |
| HC | TRP33 | HD1 | 3.21 | H12 |
| HC | TRP33 | HE1 | 3.78 | H12 |
| HC | TRP33 | CD1 | 3.48 | H11 |
| HC | TRP33 | CG | 3.88 | H11 |
| HC | TRP33 | HB2 | 3.60 | H11 |
| HC | TRP33 | HD1 | 3.38 | H11 |
| HC | TRP33 | NE1 | 3.95 | H11 |
| HC | TRP33 | HE1 | 3.55 | H10 |
| HC | TRP33 | HZ2 | 3.96 | H10 |
| HC | TRP33 | NE1 | 3.62 | C6 |
| HC | TRP33 | HZ2 | 3.79 | C6 |
| HC | TRP33 | HE1 | 3.43 | C6 |
| HC | TRP33 | CZ2 | 3.93 | C6 |
| HC | TRP33 | CE2 | 3.80 | C6 |
| HC | TRP33 | NE1 | 3.46 | C5 |
| HC | TRP33 | HE1 | 3.52 | C5 |
| HC | TRP33 | HD1 | 3.39 | C5 |
| HC | TRP33 | CD1 | 3.37 | C5 |
| HC | TRP33 | CZ3 | 3.94 | C3 |
| HC | TRP33 | CZ2 | 3.88 | C3 |
| HC | TRP33 | CH2 | 3.90 | C3 |
| HC | TRP33 | CE3 | 3.92 | C3 |
| HC | TRP33 | CE2 | 3.83 | C3 |
| HC | TRP33 | CD2 | 3.88 | C3 |
| HC | TRP33 | NE1 | 3.53 | C2 |
| HC | TRP33 | HE1 | 3.64 | C2 |
| HC | TRP33 | CE2 | 3.75 | C2 |
| HC | TRP33 | CD1 | 3.83 | C2 |
| HC | TRP33 | HE1 | 3.30 | C18 |
| HC | TRP33 | NE1 | 3.60 | C13 |
| HC | TRP33 | HE1 | 3.00 | C13 |
| HC | TRP33 | HD1 | 4.00 | C13 |
| HC | TRP33 | HZ3 | 3.90 | C12 |
| HC | TRP33 | HE3 | 3.23 | C12 |
| HC | TRP33 | CZ3 | 3.78 | C12 |
| HC | TRP33 | CE3 | 3.40 | C12 |
| HC | TRP33 | CD2 | 3.98 | C12 |
| HC | TRP33 | NE1 | 3.64 | C11 |
| HC | TRP33 | HZ2 | 3.97 | C11 |
| HC | TRP33 | HE1 | 3.09 | C11 |
| HC | TRP33 | NE1 | 3.52 | C10 |
| HC | TRP33 | HE1 | 3.24 | C10 |
| HC | TRP33 | HD1 | 3.28 | C10 |
| HC | TRP33 | CD1 | 3.54 | C10 |
| HC | SER35 | OG | 3.83 | S24 |
| HC | SER35 | HB2 | 3.62 | S24 |
| HC | SER35 | HB2 | 3.55 | O20 |
| HC | TRP47 | NE1 | 3.63 | S24 |
| HC | TRP47 | HE1 | 3.38 | S24 |
| HC | TRP47 | HD1 | 3.23 | S24 |
| HC | TRP47 | CD1 | 3.55 | S24 |
| HC | TRP47 | HE1 | 3.98 | O20 |
| HC | TRP47 | HD1 | 3.36 | CL29 |
| HC | TRP47 | HB2 | 3.34 | CL29 |
| HC | TRP47 | CD1 | 3.96 | CL29 |
| HC | TRP47 | HD1 | 3.76 | C27 |
| HC | SER50 | OG | 3.46 | S24 |
| HC | SER50 | HG | 2.90 | S24 |
| HC | SER50 | OG | 2.49 | O20 |
| HC | SER50 | HG | 1.73 | O20 |
| HC | SER50 | HB2 | 3.45 | O20 |
| HC | SER50 | HB1 | 2.75 | O20 |
| HC | SER50 | CB | 3.08 | O20 |
| HC | SER50 | HG | 3.86 | N14 |
| HC | SER50 | HB1 | 3.87 | H5 |
| HC | SER50 | HG | 3.79 | H5 |
| HC | SER50 | OG | 3.98 | C19 |
| HC | SER50 | HG | 3.19 | C19 |
| HC | SER50 | OG | 3.55 | C16 |
| HC | SER50 | HG | 2.70 | C16 |
| HC | SER50 | HB1 | 3.97 | C16 |
| HC | SER53 | OG | 3.73 | H13 |
| HC | VAL99 | HG23 | 3.34 | O9 |
| HC | VAL99 | HG22 | 3.25 | O9 |
| HC | VAL99 | HG21 | 3.13 | O9 |
| HC | VAL99 | CG2 | 3.48 | O9 |
| HC | VAL99 | HG23 | 2.87 | O8 |
| HC | VAL99 | HG22 | 3.89 | O8 |
| HC | VAL99 | HG21 | 3.51 | O8 |
| HC | VAL99 | CG2 | 3.63 | O8 |
| HC | VAL99 | HG23 | 3.72 | O20 |
| HC | VAL99 | HG13 | 2.97 | O20 |
| HC | VAL99 | HG23 | 2.74 | N14 |
| HC | VAL99 | HG21 | 4.00 | N14 |
| HC | VAL99 | HG13 | 3.66 | N14 |
| HC | VAL99 | HB | 4.00 | N14 |
| HC | VAL99 | CG2 | 3.75 | N14 |
| HC | VAL99 | HG23 | 3.76 | H5 |
| HC | VAL99 | CG1 | 3.92 | H4 |
| HC | VAL99 | CG2 | 3.20 | H4 |
| HC | VAL99 | HG13 | 3.17 | H4 |
| HC | VAL99 | HG21 | 3.82 | H4 |
| HC | VAL99 | HG22 | 3.43 | H4 |
| HC | VAL99 | HG23 | 2.18 | H4 |
| HC | VAL99 | CG2 | 3.89 | H3 |
| HC | VAL99 | HG21 | 3.86 | H3 |
| HC | VAL99 | HG23 | 3.00 | H3 |
| HC | VAL99 | HG22 | 3.38 | H11 |
| HC | VAL99 | HB | 3.87 | H1 |
| HC | VAL99 | HG23 | 3.96 | H1 |
| HC | VAL99 | HG23 | 3.54 | C7 |
| HC | VAL99 | HG23 | 3.16 | C4 |
| HC | VAL99 | HG22 | 3.55 | C4 |
| HC | VAL99 | HG21 | 3.57 | C4 |
| HC | VAL99 | CG2 | 3.67 | C4 |
| HC | VAL99 | HB | 3.91 | C25 |
| HC | VAL99 | HG13 | 3.92 | C19 |
| HC | VAL99 | HB | 3.83 | C19 |
| HC | VAL99 | HG23 | 3.26 | C16 |
| HC | VAL99 | HG13 | 3.20 | C16 |
| HC | VAL99 | HB | 3.80 | C16 |
| HC | VAL99 | HG23 | 2.83 | C12 |
| HC | VAL99 | HG13 | 3.90 | C12 |
| HC | VAL99 | CG2 | 3.91 | C12 |
| HC | ARG101 | N | 3.36 | O9 |
| HC | ARG101 | HN | 3.27 | O9 |
| HC | ARG101 | HA | 2.36 | O9 |
| HC | ARG101 | CA | 3.13 | O9 |
| HC | ARG101 | C | 3.33 | O9 |
| HC | ARG101 | HA | 3.93 | H11 |
| HC | ARG101 | HN | 3.87 | H11 |
| HC | ARG101 | HA | 3.46 | C4 |
| HC | ASN102 | N | 2.65 | O9 |
| HC | ASN102 | HN | 1.80 | O9 |
| HC | ASN102 | HB2 | 3.87 | O9 |
| HC | ASN102 | HA | 3.45 | O9 |
| HC | ASN102 | CA | 3.67 | O9 |
| HC | ASN102 | OD1 | 3.05 | O8 |
| HC | ASN102 | N | 3.07 | O8 |
| HC | ASN102 | HN | 2.53 | O8 |
| HC | ASN102 | HB2 | 3.41 | O8 |
| HC | ASN102 | HA | 2.43 | O8 |
| HC | ASN102 | CG | 3.57 | O8 |
| HC | ASN102 | CB | 3.57 | O8 |
| HC | ASN102 | CA | 3.13 | O8 |
| HC | ASN102 | OD1 | 3.04 | N14 |
| HC | ASN102 | HA | 3.01 | N14 |
| HC | ASN102 | HN | 3.82 | N1 |
| HC | ASN102 | CG | 3.33 | H6 |
| HC | ASN102 | HA | 3.56 | H6 |
| HC | ASN102 | HD21 | 3.98 | H6 |
| HC | ASN102 | OD1 | 2.56 | H6 |
| HC | ASN102 | C | 3.88 | H3 |
| HC | ASN102 | CA | 3.20 | H3 |
| HC | ASN102 | CB | 3.79 | H3 |
| HC | ASN102 | CG | 3.25 | H3 |
| HC | ASN102 | HA | 2.15 | H3 |
| HC | ASN102 | HN | 3.80 | H3 |
| HC | ASN102 | N | 4.00 | H3 |
| HC | ASN102 | OD1 | 2.16 | H3 |
| HC | ASN102 | O | 3.58 | H2 |
| HC | ASN102 | C | 2.91 | H1 |
| HC | ASN102 | CA | 3.16 | H1 |
| HC | ASN102 | CB | 3.96 | H1 |
| HC | ASN102 | CG | 3.64 | H1 |
| HC | ASN102 | HA | 2.47 | H1 |
| HC | ASN102 | O | 2.93 | H1 |
| HC | ASN102 | OD1 | 2.90 | H1 |
| HC | ASN102 | OD1 | 3.19 | C7 |
| HC | ASN102 | HN | 3.98 | C7 |
| HC | ASN102 | HA | 3.47 | C7 |
| HC | ASN102 | CG | 3.97 | C7 |
| HC | ASN102 | N | 3.25 | C4 |
| HC | ASN102 | HN | 2.48 | C4 |
| HC | ASN102 | HB2 | 3.89 | C4 |
| HC | ASN102 | HA | 3.37 | C4 |
| HC | ASN102 | CA | 3.85 | C4 |
| HC | ASN102 | OD1 | 3.67 | C25 |
| HC | ASN102 | O | 3.81 | C25 |
| HC | ASN102 | HA | 3.50 | C25 |
| HC | ASN102 | C | 3.89 | C25 |
| HC | ASN102 | OD1 | 3.70 | C12 |
| HC | ASN102 | HA | 3.73 | C12 |
| HC | TYR103 | HA | 3.01 | H2 |
| HC | TYR103 | CA | 3.96 | H1 |
| HC | TYR103 | HA | 3.43 | H1 |
| HC | TYR103 | N | 3.50 | H1 |
| HC | TYR103 | HA | 3.68 | C28 |
| HC | TYR103 | HA | 3.88 | C25 |
| HC | LEU104 | HD23 | 3.04 | S24 |
| HC | LEU104 | HD13 | 3.78 | S24 |
| HC | LEU104 | HG | 3.05 | H2 |
| HC | LEU104 | HG | 3.60 | CL29 |
| HC | LEU104 | HD23 | 3.97 | CL29 |
| HC | LEU104 | HD13 | 2.83 | CL29 |
| HC | LEU104 | HD12 | 3.44 | CL29 |
| HC | LEU104 | CD1 | 3.53 | CL29 |
| HC | LEU104 | HG | 3.16 | C28 |
| HC | LEU104 | HD23 | 3.42 | C28 |
| HC | LEU104 | HD21 | 3.95 | C28 |
| HC | LEU104 | CG | 3.99 | C28 |
| HC | LEU104 | CD2 | 3.97 | C28 |
| HC | LEU104 | HG | 3.32 | C27 |
| HC | LEU104 | HD23 | 3.14 | C27 |
| HC | LEU104 | HD13 | 3.26 | C27 |
| HC | LEU104 | CG | 3.91 | C27 |
| HC | LEU104 | CD2 | 3.91 | C27 |
| HC | LEU104 | CD1 | 3.94 | C27 |
| HC | LEU104 | HG | 3.92 | C25 |
| HC | LEU104 | HD23 | 3.53 | C25 |
| HC | LEU104 | HD21 | 3.88 | C25 |
| HC | LEU104 | HD23 | 3.33 | C19 |

In summary, Fab M18-G08-G-DKTHT recognizes rivaroxaban by the following residues:

### Light Chain:

### Asn35 [L-CDR1], Tyr37, Gln90 [L-CDR3], Trp99 [L-CDR3], Phe101

### Heavy Chain:

### Ser31 [H-CDR1], Trp33 [H-CDR1], Ser35 [H-CDR1], Trp47, Ser50 [H-CDR2, Va199 [H-CDR3], Trp100 [H-CDR3], Arg101 [H-CDR3], Asn102 [H-CDR3], Tyr103 [H-CDR3], Leu104 [H-CDR3]

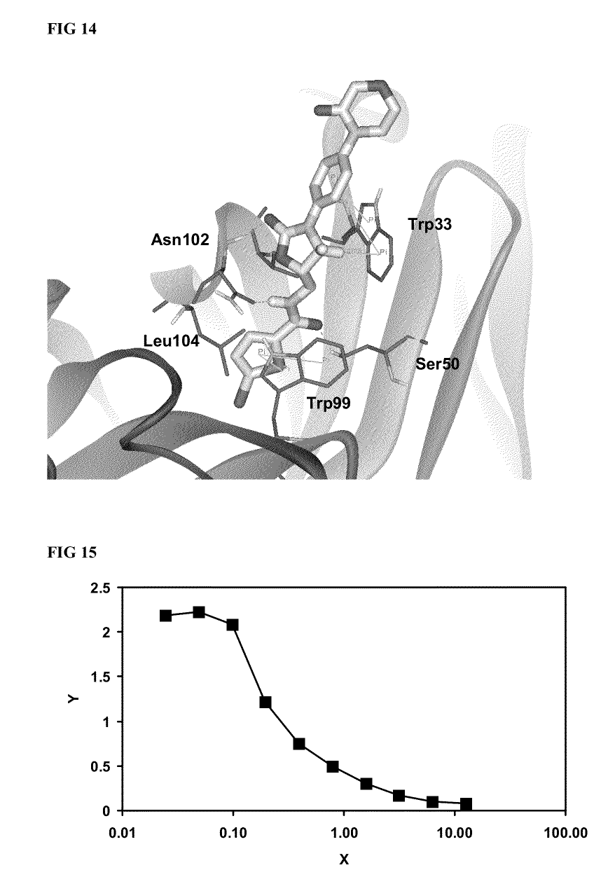

The chlorthiophene moiety of rivaroxaban interacts via π-stacking to Trp99 (L-CDR3) and via hydrophobic stacking to Leu104 (H-CDR3). The central amide of rivaroxaban is hydrogen bonded to side chains of Ser50 (H-CDR2) and Asn102 (H-CDR3). The carbonyl oxygen of the oxazole of rivaroxaban is hydrogen bonded to main chain amide of Asn102 (H-CDR3). All these interactions described can be transferred to formula 1.
The phenyl ring of rivaroxaban interacts via π-stacking to Trp33 (H-CDR1). These interaction can be transferred to formula 2.
FIG. 13 depicts a cartoon representation of the Fab M18-G08-G-DKTHT in complex with rivaroxaban shown in sticks. FIG. 14 depicts binding and interaction of Fab M18-G08-G-DKTHT with rivaroxaban—

### Example 22

### Determination and Quantification of Rivaroxaban Content by Competition ELISA

PBST: 1×PBS supplemented with 0.05% Tween20 (Sigma, P7949)
PBST-MP3%: PBST supplemented with 3% milkpowder (Cell Signaling, 9999)

To determine the content of rivaroxaban in a given sample a competition ELISA was established. Briefly, MTP plates (Greiner, No. 655990) pre-coated with streptavidin were incubated with 100 nM compound from Example 1K for 1 h at RT. After washing with PBST, plates were blocked with PBST-MP3% for 1 h at room temperature and the washing step was repeated. For the competition step samples containing various amounts of rivaroxaban serially diluted in PBS were mixed with Fab M18-G08-G-DKTHT at a final concentration of 2.5 μg/ml, incubated at RT for 1 h and subsequently transferred to the pre-treated wells. After incubation and washing with PBST, detection of the residually bound Fabs was performed with an anti-hIgG (Fab-specific) coupled to HRP (Sigma; A 0293). Color reaction was developed by addition of 100 μl TMB (Invitrogen, 2023) and stopped after 5-15 min by adding 100 μl H2504 (0.25 M; Merck, 1120801000). Colorimetric reaction was recorded at 450 nM in a plate reader (Tecan).

As depicted in FIG. 15 a dose-dependent decrease of the signal could be detected.

### Example 23

### Activity of Apixaban and Dabigatran in a Thrombin Generation Assay in the Presence of Fab-Antidote

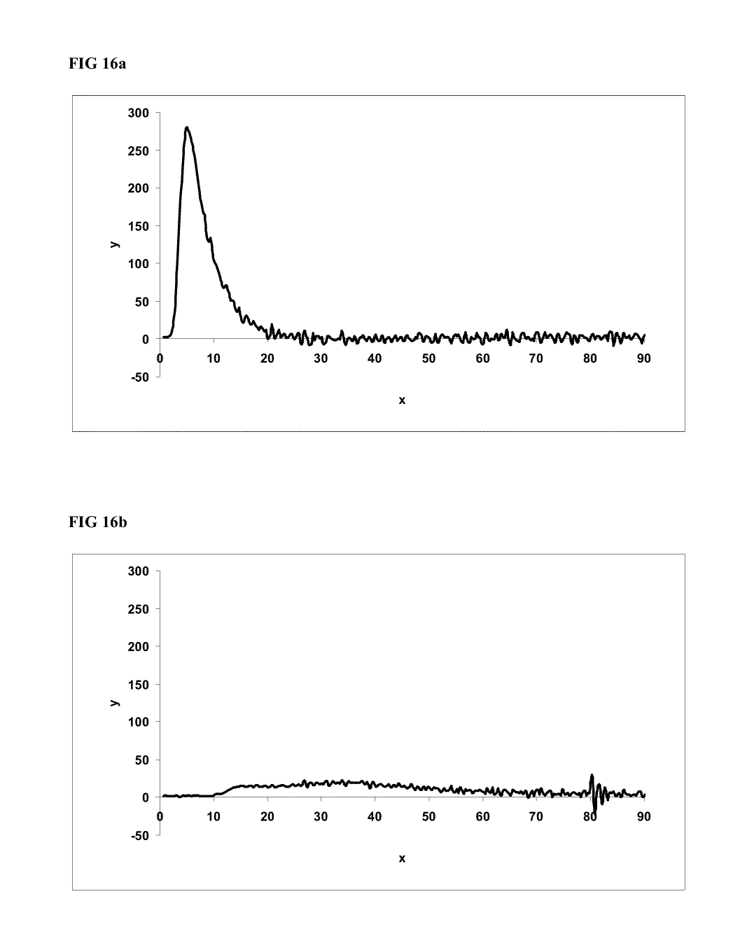

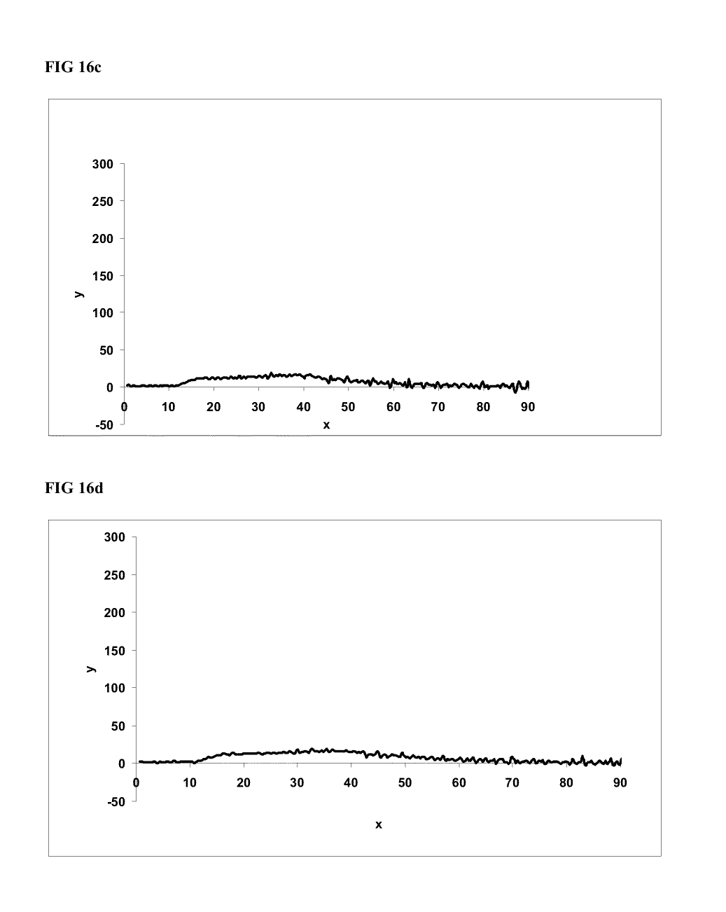

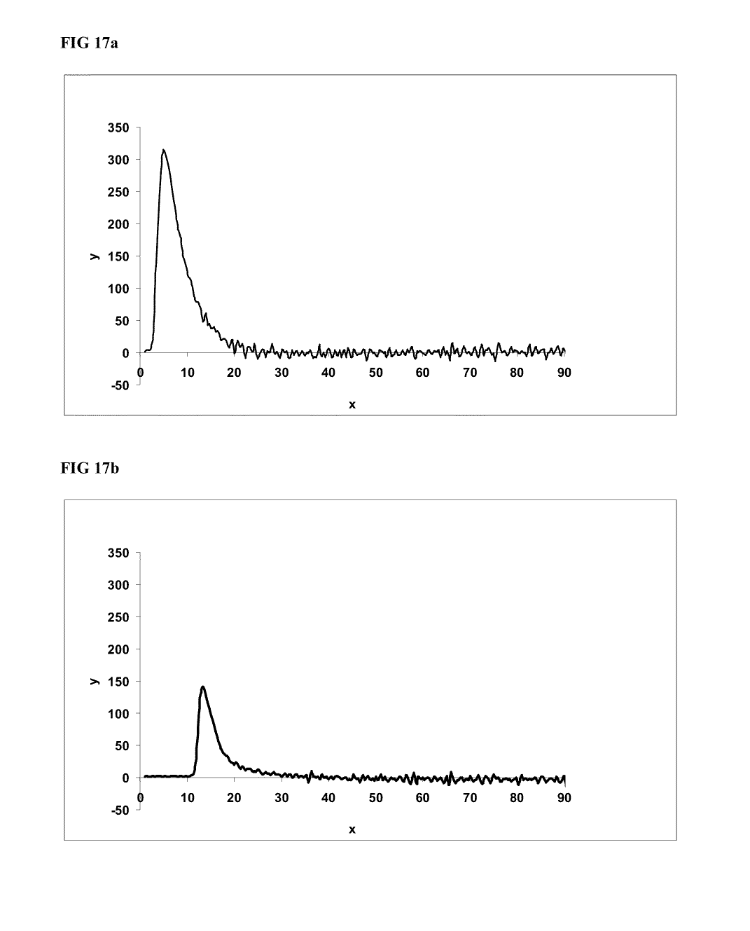

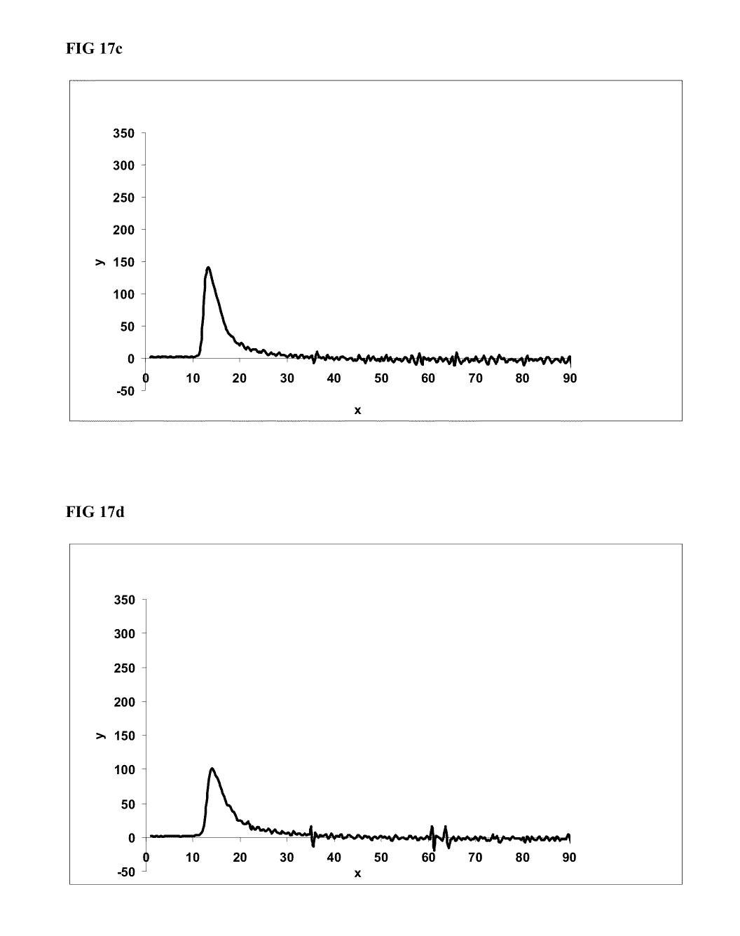

Thrombin generation assays were performed essentially as described in Example 8. Experiments were performed in the presence of either 3 μM apixaban (FIG. 16) or 0.75 μM dabigatran (FIG. 17), respectively. No effect on thrombin generation was detectable when either Fab antidote alone (final concentration of 1.43 μM and 0.72 μM, respectively) or Fab antidote in combination with rivaroxaban (final concentration of 0.1 μM) was added.

### Lit.:

Benton, T. et al. (2002) Cytotechnology 2002; 38: 43-46)
Cicortas Gunnarsson et al., Protein Eng Des Sel 2004; 17 (3): 213-21
Emsley P. et al. (2010) Acta Cryst. D66:486-501
Evans P. R, (2005) Acta Cryst. D62, 72-82
Garcia, D. et al., Blood 2010; 115(1):15-20 G
Gebauer M. et al., Curr. Opinion in Chem. Biol. 2009; 13:245-255
Guertin et al., Current Medicinal Chemistry 2007, 14, 2471-2781
Long et al. F (2008) Acta Cryst. D64, 125-132
Murshudo vG. N. et al. (1997) Acta Cryst. D53, 240-255
Kabsch, W. (2010) Acta Cryst. D66, 125-132
Keefe A D., et al., Nat. Rev. Drug Discov. 2010; 9:537-550)
Levi, M. et al., N Engl J. Med. 2010; 363(19):1791-800.
Nuttall S. D. et al., Curr. Opinion in Pharmacology 2008; 8:608-617
Perzborn E. et al., Nat. Rev. Drug Discov. 2011; 10(1):61-75
Romualdi et al.; Curr. Pharm. Des. 2010; 16(31):3478-82
Schlehuber S, and Skerra A., Drug Discov. Today 2005; 10 (1):23-33)
Schuhmacher J, et al. J Pharm Sci. 2004; 93(4):816-30
Sheedy, C. et al., Biotech Adv 2007; 25:333-52.
Söderling et al., Nature Biotech. 2000, 18:853-856
Sorbera et al. Drugs of the Future 2005, 30(9):877-885
Vaughan et al, Nat. Biotech. 1996; 14 (3):309-314
Vimekäs B. et al., Nucl. Acids Res. 1994, 22: 5600
WO 2009/042962 A2
WO 97/08320

1-2. (canceled)

3. An isolated antibody or antigen-binding fragment thereof or antibody mimetics which binds an anticoagulant or neutralizes the anti-coagulant activity of said anticoagulant in vitro and/or in vivo.

4. An isolated antibody or antigen-binding fragment thereof or antibody mimetics according to claim 3, wherein the anticoagulant has a molecular weight of less than 5000 Da.

5. An isolated antibody or antigen-binding fragment thereof or antibody mimetics according to claim 3, wherein the anticoagulant is a Factor Xa inhibitor or a thrombin inhibitor.

6. An isolated antibody or antigen-binding fragment thereof or antibody mimetics according to claim 3, wherein the FXa inhibitor is a compound comprising a group of the formula 1, apixaban, betrixaban, razaxaban, edoxaban, otamixaban or YM-150 or wherein the thrombin inhibitor is dabigatran.

7. An isolated antibody or antigen-binding fragment thereof or antibody mimetics according to claim 3, wherein the anti-coagulant is rivaroxaban.

8. An isolated antibody or antigen-binding fragment thereof according to claim 3, wherein the antibody sequence comprises the variable heavy chain CDR sequences and the variable light chain CDR sequences of an antibody of table 1.

9. The antibody or antigen binding fragment according to claim 3, comprising
the variable heavy chain CDR sequences as presented by SEQ ID NO: 263-265 and
the variable light chain CDR sequences presented by SEQ ID NO: 266-268, or
the variable heavy chain CDR sequences as presented by SEQ ID NO: 251-253 and
the variable light chain CDR sequences presented by SEQ ID NO: 254-256, or
the variable heavy chain CDR sequences as presented by SEQ ID NO: 221-223 and
the variable light chain CDR sequences presented by SEQ ID NO: 224-226.

10. An isolated antibody or antigen-binding fragment thereof according to claim 3, wherein the antibody sequence comprises the variable heavy chain sequence and the variable light chain sequence of an antibody depicted in table 1.

11. The antibody or antigen binding fragment according to claim 3, comprising
a heavy chain fragment sequence as presented by SEQ ID NO: 489 and a light chain sequence as presented by SEQ ID NO: 490, or
a variable heavy chain sequence as presented by SEQ ID NO: 217 and a variable light chain sequence as presented by SEQ ID NO: 218, or
a variable heavy chain sequence as presented by SEQ ID NO: 117 and a variable light chain sequence as presented by SEQ ID NO: 118, or
a variable heavy chain sequence as presented by SEQ ID NO: 207 and a variable light chain sequence as presented by SEQ ID NO: 208, or
a heavy chain fragment sequence as presented by SEQ ID NO: 493 and a light chain sequence as presented by SEQ ID NO: 494.

12. An isolated antibody or antigen-binding fragment thereof or antibody mimetics that competes in binding with an antibody or antigen-binding fragment of claims 8-11.

13. The antibody or antigen-binding fragment according to claim 12, wherein the amino acid sequence of the antibody or antigen-binding fragment is at least 50%, 55%, 60% 70%, 80%, 90, or 95% identical to at least one CDR sequence depicted in table 1, or at least 50%, 60%, 70%, 80%, 90%, 92% or 95% identical to at least one VH or VL sequence depicted in table 1.

14. The antibody or antigen-binding fragment according to claim 12, wherein the amino acid sequence of the antibody or antigen-binding fragment is at least 50%, 55%, 60% 70%, 80%, 90, or 95% identical to at least one CDR sequence of M18-G08-G, or at least 50%, 60%, 70%, 80%, 90%, 92% or 95% identical to the VH or VL sequence of M18-G08-G.

15. The antibody or antigen-binding fragment according to claim 12, wherein the antibody or antigen-binding fragment thereof comprises at least one of the heavy chain CDR sequences that conforms to the consensus sequences SEQ ID NO: 497 or SEQ ID NO: 502 (CDR H1), SEQ ID NO: 222 or SEQ ID NO: 503 (CDR H2), or SEQ ID NO: 498 or SEQ ID NO: 504 (CDR H3), and/or at least one of the light chain CDR sequences that conform to the consensus sequences of SEQ ID NO: 499 or SEQ ID NO: 505 (CDR L1), SEQ ID NO: 500 or SEQ ID NO: 506 (CDR L2), or SEQ ID NO: 501 or SEQ ID NO: 507 (CDR L3).

16. The antibody or antigen-binding fragment according to claim 12,
wherein the antibody or antigen-binding fragment thereof comprises the heavy chain CDR sequences conforming to SEQ ID NO: 497 (CDR H1), SEQ ID NO: 222 (CDR H2) and SEQ ID NO: 498 (CDR H3), and the light chain CDR sequences conforming to SEQ ID NO: 499 (CDR L1), SEQ ID NO: 500 (CDR L2) and SEQ ID NO: 501 (CDR L3), or
wherein the antibody or antigen-binding fragment thereof comprises the heavy chain CDR sequences conforming to SEQ ID NO: 502 (CDR H1), SEQ ID NO: 503 (CDR H2) and SEQ ID NO: 504 (CDR H3), and the light chain CDR sequences conforming to SEQ ID NO: 505 (CDR L1), SEQ ID NO: 506 (CDR L2) and SEQ ID NO: 507 (CDR L3).

17. The antibody or antigen-binding fragment according to claim 12, wherein the antibody or antigen-binding fragment comprises at least one CDR sequence or at least one variable heavy chain or light chain sequence as depicted in table 1.

18. An antigen-binding fragment according to claim 3, wherein the fragment is a Fab fragment.

19. An antibody or antigen-binding fragment according to claim 3, wherein the antibody or fragment is monoclonal.

20. An antibody or antigen-binding fragment according to claim 3, wherein the antibody or fragment is human, humanized or chimeric.

21. An isolated polynucleotide sequence encoding an antibody or antigen-binding fragment thereof or antibody mimetics according to claim 3.

22. A vector comprising a polynucleotide of claim 21.

23. A host cell comprising a polynucleotide sequence according to claim 21.

24-26. (canceled)

27. A pharmaceutical composition comprising an antibody or antigen-binding fragment thereof, or an antibody mimetic of claim 3.

28-33. (canceled)

34. A method of treatment for the normalization of an anti-coagulated status induced by an anticoagulant using a pharmaceutical composition according to claim 27.

35. A method of treatment for the normalization of an anti-coagulated status induced by a FXa inhibitor using a pharmaceutical composition according to claim 27.

36. A method of treatment for the normalization of an anti-coagulated status induced by a compound comprising a group of the formula 1 using a pharmaceutical composition according to claim 27.

37. A method of treatment for the normalization of an anti-coagulated status induced by rivaroxaban using a pharmaceutical composition according to claim 28.

38. A compound of Example 1K or Example 1L.

39. (canceled)

40. A diagnostic kit comprising a compound of Example 1K or Example 1L and/or an antibody or antigen-binding fragment thereof according to claims 1-20.

41. (canceled)
